SEQUENCE LISTING (117 sequences)

SEQ ID NO: 1 - RNA, 14 nt, Artificial Sequence
  Synthetic polynucleotide
accacuuccuuucg

SEQ ID NO: 2 - RNA, 10 nt, Artificial Sequence
  Synthetic polynucleotide
acauacaaag

SEQ ID NO: 3 - RNA, 18 nt, Artificial Sequence
  Synthetic polynucleotide
auauuacuucacacaaug

SEQ ID NO: 4 - RNA, 12 nt, Artificial Sequence
  Synthetic polynucleotide
gccuccccccag

SEQ ID NO: 5 - RNA, 24 nt, Artificial Sequence
  Synthetic polynucleotide
ccccuccuccccuuccugcacccg

SEQ ID NO: 6 - RNA, 141 nt, Artificial Sequence
  Synthetic polynucleotide
caaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 7 - DNA, 16 bp, Artificial Sequence
  Synthetic polynucleotide
  modified_base (1)..(12): may be modified by a 2' O-methylated base
cauucucuuauutccc

SEQ ID NO: 8 - DNA, 16 bp, Artificial Sequence
  Synthetic polynucleotide
  modified_base (1)..(11): may be modified by a 2' O-methylated base
  modified_base (16)..(16): may be modified by a 2' O-methylated base
cauucucuuauttccc

SEQ ID NO: 9 - DNA, 16 bp, Artificial Sequence
  Synthetic polynucleotide
  modified_base (1)..(10): may be modified by a 2' O-methylated base
  modified_base (15)..(16): may be modified by a 2' O-methylated base
cauucucuuatttccc

SEQ ID NO: 10 - DNA, 16 bp, Artificial Sequence
  Synthetic polynucleotide
  modified_base (1)..(9): may be modified by a 2' O-methylated base
  modified_base (14)..(16): may be modified by a 2' O-methylated base
cauucucuuatttccc

SEQ ID NO: 11 - DNA, 16 bp, Artificial Sequence
  Synthetic polynucleotide
  modified_base (1)..(8): may be modified by a 2' O-methylated base
  modified_base (13)..(16): may be modified by a 2' O-methylated base
cauucucutattuccc

SEQ ID NO: 12 - DNA, 16 bp, Artificial Sequence
  Synthetic polynucleotide
  modified_base (1)..(7): may be modified by a 2' O-methylated base
  modified_base (12)..(16): may be modified by a 2' O-methylated base
cauucucttatuuccc

SEQ ID NO: 13 - DNA, 16 bp, Artificial Sequence
  Synthetic polynucleotide
  modified_base (1)..(6): may be modified by a 2' O-methylated base
  modified_base (11)..(16): may be modified by a 2' O-methylated base
cauucucttauuuccc

SEQ ID NO: 14 - DNA, 16 bp, Artificial Sequence
  Synthetic polynucleotide
  modified_base (1)..(5): may be modified by a 2' O-methylated base
  modified_base (10)..(16): may be modified by a 2' O-methylated base
cauuctcttauuuccc

SEQ ID NO: 15 - DNA, 16 bp, Artificial Sequence
  Synthetic polynucleotide
  modified_base (1)..(4): may be modified by a 2' O-methylated base
  modified_base (9)..(16): may be modified by a 2' O-methylated base
cauuctctuauuuccc

SEQ ID NO: 16 - DNA, 16 bp, Artificial Sequence
  Synthetic polynucleotide
  modified_base (1)..(3): may be modified by a 2' O-methylated base
  modified_base (8)..(16): may be modified by a 2' O-methylated base
cautctcuuauuuccc

SEQ ID NO: 17 - DNA, 16 bp, Artificial Sequence
  Synthetic polynucleotide
  modified_base (1)..(2): may be modified by a 2' O-methylated base
  modified_base (7)..(16): may be modified by a 2' O-methylated base
cattctcuuauuuccc

SEQ ID NO: 18 - DNA, 16 bp, Artificial Sequence
  Synthetic polynucleotide
  modified_base (1)..(1): may be modified by a 2' O-methylated base
  modified_base (6)..(16): may be modified by a 2' O-methylated base
cattcucuuauuuccc

SEQ ID NO: 19 - DNA, 9 bp, Artificial Sequence
  Synthetic polynucleotide
  modified_base (1)..(1): may be modified by a 2' O-methylated base
  modified_base (6)..(9): may be modified by a 2' O-methylated base
utattuccc

SEQ ID NO: 20 - DNA, 9 bp, Artificial Sequence
  Synthetic polynucleotide
  modified_base (1)..(2): may be modified by a 2' O-methylated base
  modified_base (7)..(9): may be modified by a 2' O-methylated base
uuatttccc

SEQ ID NO: 21 - DNA, 9 bp, Artificial Sequence
  Synthetic polynucleotide
uttttuccc

SEQ ID NO: 22 - DNA, 9 bp, Artificial Sequence
  Synthetic polynucleotide
uuatttccc

SEQ ID NO: 23 - DNA, 23 bp, Artificial Sequence
  Synthetic polynucleotide
  modified_base (1)..(9): may be modified by a 2' O-methylated base
  modified_base (10)..(13): may be modified by a 2'-deoxyribonucleotide
  modified_base (14)..(23): may be modified by a 2' O-methylated base
uuuuuuuuutgccgcccacucag

SEQ ID NO: 24 - RNA, 14 nt, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
gccgcccacucaga

SEQ ID NO: 25 - RNA, 14 nt, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
ccacucagacuuua

SEQ ID NO: 26 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
cagactttauucaa

SEQ ID NO: 27 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
uuuautcaaagacc

SEQ ID NO: 28 - DNA, 10 bp, Artificial Sequence
  Synthetic polynucleotide
  modified_base (1)..(2): may be modified by a 2' O-methylated base
  modified_base (3)..(7): may be modified by a 2'-deoxyribonucleotide
  modified_base (8)..(10): may be modified by a 2' O-methylated base
gactttauuc

SEQ ID NO: 29 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  misc_feature (14)..(14): may be modified by 36FAM
cagactttauucaa

SEQ ID NO: 30 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  modified_base (14)..(14): may be modified by 3Sp13
cagactttauucaa

SEQ ID NO: 31 - DNA, 14 bp, Artificial Sequence
  Synthetic polynucleotide
  modified_base (1)..(5): may be modified by a 2' O-methylated base
  misc_feature (6)..(6): is 5-nitroindole
  modified_base (6)..(9): may be modified by a 2'-deoxyribonucleotide
  modified_base (10)..(14): may be modified by a 2' O-methylated base
cagacnttauucaa

SEQ ID NO: 32 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (7)..(7): is 5-nitroindole
  modified_base (10)..(14): may be modified by a 2' O-methylated base
cagactntauucaa

SEQ ID NO: 33 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (8)..(8): is 5-nitroindole
  modified_base (10)..(14): may be modified by a 2' O-methylated base
cagacttnauucaa

SEQ ID NO: 34 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (9)..(9): is 5-nitroindole
  modified_base (10)..(14): may be modified by a 2' O-methylated base
cagactttnuucaa

SEQ ID NO: 35 - RNA, 14 nt, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (6)..(9): 5-nitroindole
  modified_base (10)..(14): may be modified by a 2' O-methylated base
cagacnnnnuucaa

SEQ ID NO: 36 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (6)..(6): I
  misc_feature (6)..(6): n is a, c, g, t or u
  modified_base (10)..(14): may be modified by a 2' O-methylated base
cagacnttauucaa

SEQ ID NO: 37 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (7)..(7): I
  misc_feature (7)..(7): n is a, c, g, t or u
  modified_base (10)..(14): may be modified by a 2' O-methylated base
cagactntauucaa

SEQ ID NO: 38 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (8)..(8): I
  misc_feature (8)..(8): n is a, c, g, t or u
  modified_base (10)..(14): may be modified by a 2' O-methylated base
cagacttnauucaa

SEQ ID NO: 39 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (9)..(9): I
  misc_feature (9)..(9): n is a, c, g, t or u
  modified_base (10)..(14): may be modified by a 2' O-methylated base
cagactttnuucaa

SEQ ID NO: 40 - DNA, 14 bp, Artificial Sequence
  Synthetic polynucleotide
  modified_base (1)..(3): may be modified by a 2' O-methylated base
  modified_base (4)..(4): may be modified by a 2'-deoxyribonucleotide
  misc_feature (4)..(4): is 5-nitroindole
  modified_base (5)..(5): may be modified by a 2' O-methylated base
  modified_base (6)..(9): may be modified by a 2'-deoxyribonucleotide
  modified_base (10)..(14): may be modified by a 2' O-methylated base
cagnctttauucaa

SEQ ID NO: 41 - DNA, 14 bp, Artificial Sequence
  Synthetic polynucleotide
  modified_base (1)..(4): may be modified by a 2' O-methylated base
  modified_base (5)..(9): may be modified by a 2'-deoxyribonucleotide
  misc_feature (5)..(5): is 5-nitroindole
  modified_base (10)..(14): may be modified by a 2' O-methylated base
cagantttauucaa

SEQ ID NO: 42 - DNA, 14 bp, Artificial Sequence
  Synthetic polynucleotide
  modified_base (1)..(3): may be modified by a 2' O-methylated base
  modified_base (4)..(9): may be modified by a 2'-deoxyribonucleotide
  misc_feature (4)..(5): 5-nitroindole
  modified_base (10)..(14): may be modified by a 2' O-methylated base
cagnntttauucaa

SEQ ID NO: 43 - DNA, 14 bp, Artificial Sequence
  Synthetic polynucleotide
  modified_base (1)..(5): may be modified by a 2' O-methylated base
  modified_base (6)..(7): may be modified by a 2'-deoxyribonucleotide
  modified_base (8)..(14): may be modified by a 2' O-methylated base
cagacttuauucaa

SEQ ID NO: 44 - DNA, 14 bp, Artificial Sequence
  Synthetic polynucleotide
  misc_feature (1)..(3): 5-nitroindole
  modified_base (1)..(3): may be modified by a 2'-deoxyribonucleotide
  modified_base (4)..(5): may be modified by a 2' O-methylated base
  modified_base (6)..(14): may be modified by a 2'-deoxyribonucleotide
  misc_feature (8)..(14): 5-nitroindole
nnnacttnnnnnnn

SEQ ID NO: 45 - RNA, 95 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - RNA, 60 nt, Artificial Sequence
  Synthetic polynucleotide
aaaaaaaaaaaaaaaaaaaaaaaaaaaaaaaaaaaaaaaaaaaaaaaaaaaaaaaaaaaa

SEQ ID NO: 47 - RNA, 140 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - RNA, 100 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - RNA, 17 nt, Artificial Sequence
  Synthetic polynucleotide
ggagagagaugggugcg

SEQ ID NO: 50 - DNA, 31 bp, Artificial Sequence
  Synthetic polynucleotide
cgcacccaggctagctacaacgactctctcc

SEQ ID NO: 51 - RNA, 16 nt, Artificial Sequence
  Synthetic polynucleotide
gggaaauaagagaaug

SEQ ID NO: 52 - DNA, 16 bp, Artificial Sequence
  Synthetic polynucleotide
  modified_base (1)..(3): may be modified by a 2' O-methylated base
  modified_base (4)..(7): may be modified by a 2'-deoxyribonucleotide
  modified_base (8)..(16): may be modified by a 2' O-methylated base
aauucucuuatttccc

SEQ ID NO: 53 - DNA, 16 bp, Artificial Sequence
  Synthetic polynucleotide
  modified_base (1)..(4): may be modified by a 2' O-methylated base
  modified_base (5)..(8): may be modified by a 2'-deoxyribonucleotide
  modified_base (9)..(16): may be modified by a 2' O-methylated base
aauucucutattuccc

SEQ ID NO: 54 - DNA, 16 bp, Artificial Sequence
  Synthetic polynucleotide
  modified_base (1)..(7): may be modified by a 2' O-methylated base
  modified_base (8)..(11): may be modified by a 2'-deoxyribonucleotide
  modified_base (12)..(16): may be modified by a 2' O-methylated base
aauucucttatuuccc

SEQ ID NO: 55 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  misc_feature (14)..(14): may be modified by 36FAM
cagactttauucaa

SEQ ID NO: 56 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  modified_base (14)..(14): may be modified by 3Sp13
cagactttauucaa

SEQ ID NO: 57 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
cagactttauucaa

SEQ ID NO: 58 - DNA, 10 bp, Artificial Sequence
  Synthetic polynucleotide
  modified_base (1)..(2): may be modified by a 2' O-methylated base
  modified_base (3)..(7): may be modified by a 2'-deoxyribonucleotide
  modified_base (8)..(10): may be modified by a 2' O-methylated base
gactttauuc

SEQ ID NO: 59 - RNA, 117 nt, Artificial Sequence
  Synthetic polynucleotide
gucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 60 - RNA, 94 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 61 - RNA, 96 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 62 - RNA, 97 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 63 - RNA, 98 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 64 - RNA, 99 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 65 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
cagactttauucaa

SEQ ID NO: 66 - DNA, 10 bp, Artificial Sequence
  Synthetic polynucleotide
  modified_base (1)..(2): may be modified by a 2' O-methylated base
  modified_base (3)..(7): may be modified by a 2'-deoxyribonucleotide
  modified_base (8)..(10): may be modified by a 2' O-methylated base
cactttauuc

SEQ ID NO: 67 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  misc_feature (14)..(14): may be modified by 36FAM
cagactttauucaa

SEQ ID NO: 68 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  modified_base (14)..(14): may be modified by 3Sp13
cagactttauucaa

SEQ ID NO: 69 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
cagactttauucaa

SEQ ID NO: 70 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (8)..(8): is 5-nitroindole
  modified_base (10)..(14): may be modified by a 2' O-methylated base
cagacttnauucaa

SEQ ID NO: 71 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (9)..(9): is 5-nitroindole
  modified_base (10)..(14): may be modified by a 2' O-methylated base
cagactttnuucaa

SEQ ID NO: 72 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (9)..(9): I
  misc_feature (9)..(9): n is a, c, g, t or u
  modified_base (10)..(14): may be modified by a 2' O-methylated base
cagactttnuucaa

SEQ ID NO: 73 - DNA, 14 bp, Artificial Sequence
  Synthetic polynucleotide
  modified_base (1)..(3): may be modified by a 2' O-methylated base
  modified_base (4)..(4): may be modified by a 2'-deoxyribonucleotide
  misc_feature (4)..(4): is 5-nitroindole
  modified_base (5)..(5): may be modified by a 2' O-methylated base
  modified_base (6)..(9): may be modified by a 2'-deoxyribonucleotide
  modified_base (10)..(14): may be modified by a 2' O-methylated base
cagnctttauucaa

SEQ ID NO: 74 - DNA, 14 bp, Artificial Sequence
  Synthetic polynucleotide
  modified_base (1)..(4): may be modified by a 2' O-methylated base
  modified_base (5)..(9): may be modified by a 2'-deoxyribonucleotide
  misc_feature (5)..(5): is 5-nitroindole
  modified_base (10)..(14): may be modified by a 2' O-methylated base
cagantttauucaa

SEQ ID NO: 75 - DNA, 14 bp, Artificial Sequence
  Synthetic polynucleotide
  modified_base (1)..(3): may be modified by a 2' O-methylated base
  modified_base (4)..(9): may be modified by a 2'-deoxyribonucleotide
  misc_feature (4)..(5): 5-nitroindole
  modified_base (10)..(14): may be modified by a 2' O-methylated base
cagnntttauucaa

SEQ ID NO: 76 - DNA, 14 bp, Artificial Sequence
  Synthetic polynucleotide
  modified_base (1)..(5): may be modified by a 2' O-methylated base
  modified_base (6)..(7): may be modified by a 2'-deoxyribonucleotide
  modified_base (8)..(14): may be modified by a 2' O-methylated base
cagacttuauucaa

SEQ ID NO: 77 - DNA, 14 bp, Artificial Sequence
  Synthetic polynucleotide
  modified_base (1)..(5): may be modified by a 2' O-methylated base
  modified_base (6)..(7): may be modified by a 2'-deoxyribonucleotide
  modified_base (8)..(14): may be modified by a 2' O-methylated base
cagacttuauucaa

SEQ ID NO: 78 - DNA, 14 bp, Artificial Sequence
  Synthetic polynucleotide
  modified_base (1)..(3): may be modified by a 2' O-methylated base
  modified_base (4)..(4): may be modified by a 2'-deoxyribonucleotide
  misc_feature (4)..(4): is 5-nitroindole
  modified_base (5)..(5): may be modified by a 2' O-methylated base
  modified_base (6)..(9): may be modified by a 2'-deoxyribonucleotide
  modified_base (10)..(14): may be modified by a 2' O-methylated base
cagnctttauucaa

SEQ ID NO: 79 - DNA, 14 bp, Artificial Sequence
  Synthetic polynucleotide
  modified_base (1)..(4): may be modified by a 2' O-methylated base
  modified_base (5)..(9): may be modified by a 2'-deoxyribonucleotide
  misc_feature (5)..(5): is 5-nitroindole
  modified_base (10)..(14): may be modified by a 2' O-methylated base
cagantttauucaa

SEQ ID NO: 80 - DNA, 14 bp, Artificial Sequence
  Synthetic polynucleotide
  modified_base (1)..(3): may be modified by a 2' O-methylated base
  modified_base (4)..(9): may be modified by a 2'-deoxyribonucleotide
  misc_feature (4)..(5): 5-nitroindole
  modified_base (10)..(14): may be modified by a 2' O-methylated base
cagnntttauucaa

SEQ ID NO: 81 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
cagactttauucaa

SEQ ID NO: 82 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (10)..(14): may be modified by a 2' O-methylated base
  misc_feature (14)..(14): may be modified by 36FAM
cagactttauucaa

SEQ ID NO: 83 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (8)..(8): is 5-nitroindole
  modified_base (10)..(14): may be modified by a 2' O-methylated base
cagacttnauucaa

SEQ ID NO: 84 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  misc_feature (9)..(9): is 5-nitroindole
  modified_base (10)..(14): may be modified by a 2' O-methylated base
cagactttnuucaa

SEQ ID NO: 85 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (8)..(8): I
  misc_feature (8)..(8): n is a, c, g, t or u
  modified_base (10)..(14): may be modified by a 2' O-methylated base
cagacttnauucaa

SEQ ID NO: 86 - DNA, 14 bp, Artificial Sequence
  Synthetic polynucleotide
  modified_base (1)..(5): may be modified by a 2' O-methylated base
  modified_base (6)..(9): may be modified by a 2'-deoxyribonucleotide
  modified_base (9)..(9): I
  misc_feature (9)..(9): n is a, c, g, t or u
  modified_base (10)..(14): may be modified by a 2' O-methylated base
cagactttnuucaa

SEQ ID NO: 87 - RNA, 101 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 88 - RNA, 102 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 89 - RNA, 103 nt, Artificial Sequence
  Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 90 - RNA, 131 nt, Artificial Sequence
  Synthetic polynucleotide
guggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 91 - DNA, 14 bp, Artificial Sequence
  Synthetic polynucleotide
uuuautcaaagacc

SEQ ID NO: 92 - DNA, 14 bp, Artificial Sequence
  Synthetic polynucleotide
cagactttauucaa

SEQ ID NO: 93 - RNA, 14 nt, Artificial Sequence
  Synthetic polynucleotide
ccacucagacuuua

SEQ ID NO: 94 - RNA, 14 nt, Artificial Sequence
  Synthetic polynucleotide
gccgcccacucaga

SEQ ID NO: 95 - RNA, 16 nt, Artificial Sequence
  Synthetic polynucleotide
gggaaauaagagagaa

SEQ ID NO: 96 - RNA, 16 nt, Artificial Sequence
  Synthetic polynucleotide
gggaaauaagagaaug

SEQ ID NO: 97 - DNA, 16 bp, Artificial Sequence
  Synthetic polynucleotide
cccuuuauuctctuac

SEQ ID NO: 98 - RNA, 16 nt, Artificial Sequence
  Synthetic polynucleotide
gggaaauaagagagaa

SEQ ID NO: 99 - DNA, 16 bp, Artificial Sequence
  Synthetic polynucleotide
cccuuuauuctctuac

SEQ ID NO: 100 - RNA, 13 nt, Artificial Sequence
  Synthetic polynucleotide
uacuaucccaacg

SEQ ID NO: 101 - RNA, 13 nt, Artificial Sequence
  Synthetic polynucleotide
accaacaucuucg

SEQ ID NO: 102 - RNA, 344 nt, Artificial Sequence
  Synthetic polynucleotide
gggaaauaagagagaaaagaagaguaagaagaaauauaagagccaccaugggagugcacgagugucccgcgugguugugguugcugcugucgcucuugagccucccacugggacugccugugcugggggcaccacccagauugaucugcgacucacggguacuugagagguaccuucuugaagccaaagaagccgaaaacaucacaaccggaugcgccgagcacugcucccucaaugagaacauuacuguaccggauacaaaggucaauuucuaugcauggaagagaauggaaguaggacagcaggccgucgaaguguggcaggggcucgcgcuuuugucggag

SEQ ID NO: 103 - RNA, 10 nt, Artificial Sequence
  Synthetic polynucleotide
ccucccacug

SEQ ID NO: 104 - RNA, 10 nt, Artificial Sequence
  Synthetic polynucleotide
caccacccag

SEQ ID NO: 105 - RNA, 10 nt, Artificial Sequence
  Synthetic polynucleotide
uaccuucuug

SEQ ID NO: 106 - RNA, 15 nt, Artificial Sequence
  Synthetic polynucleotide
aaaacaucacaaccg

SEQ ID NO: 107 - RNA, 11 nt, Artificial Sequence
  Synthetic polynucleotide
cucccucaaug

SEQ ID NO: 108 - RNA, 10 nt, Artificial Sequence
  Synthetic polynucleotide
aacauuacug

SEQ ID NO: 109 - RNA, 12 nt, Artificial Sequence
  Synthetic polynucleotide
ucaauuucuaug

SEQ ID NO: 110 - RNA, 14 nt, Artificial Sequence
  Synthetic polynucleotide
ucaacucaucacag

SEQ ID NO: 111 - RNA, 16 nt, Artificial Sequence
  Synthetic polynucleotide
ccccuccaacuucaug

SEQ ID NO: 112 - RNA, 103 nt, Artificial Sequence; identical to SEQ ID NO: 89
  Synthetic polynucleotide
  misc_feature (98)..(103): may be absent

SEQ ID NO: 113 - RNA, 40 nt, Artificial Sequence
  Synthetic polynucleotide
aaaaaaaaaaaaaaaaaaaaaaaaaaaaaaaaaaaaaaaa

SEQ ID NO: 114 - RNA, 100 nt, Artificial Sequence; identical to SEQ ID NO: 48
  Synthetic polynucleotide
  misc_feature (95)..(100): may be absent

SEQ ID NO: 115 - DNA, 6 bp, Artificial Sequence
  Synthetic polynucleotide
  modified_base (1)..(1): may be modified by a 2' O-methylated base
  modified_base (6)..(6): may be modified by a 2' O-methylated base
utattu

SEQ ID NO: 116 - RNA, 300 nt, Artificial Sequence
  Synthetic polynucleotide
  misc_feature (11)..(300): may be absent
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 117 - RNA, 250 nt, Artificial Sequence
  Synthetic polynucleotide
  misc_feature (51)..(250): may be absent
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2023/0212645 A1
(43) Pub. Date: Jul. 6, 2023

(54) METHODS AND COMPOSITIONS FOR RNA MAPPING

(71) Applicant: ModernaTX, Inc., Cambridge, MA (US)

(72) Inventors: David Marquardt, Cambridge, MA (US); Nicholas J. Amato, Cambridge, MA (US); Edward J. Miracco, Arlington, MA (US); Tao Jiang, Quincy, MA (US); Serenus Hua, Cambridge, MA (US); Huijuan Li, Wayland, MA (US)

(73) Assignee: ModernaTX, Inc., Cambridge, MA (US)

(21) Appl. No.: 17/852,974

(22) Filed: Jun. 29, 2022

Related U.S. Application Data
(63) Continuation of application No. 16/001,765, filed on Jun. 6, 2018, now abandoned.
Application No. 16/001,765 is a continuation of application No. PCT/US2017/058591, filed on Oct. 26, 2017.
(60) Provisional application No. 62/412,932, filed on Oct. 26, 2016.

(51) Int. Cl. C12Q 1/6806; C12Q 1/6809; C12Q 1/68

(52) U.S. Cl. CPC C12Q 1/6806; C12Q 1/68; C12Q 1/6809

(57) ABSTRACT

Novel methods for identification and analysis of mRNA are provided herein. The methods may involve digestion and fingerprinting analysis.

### RELATED APPLICATIONS

This application is a continuation of U.S. Application Serial No. 16/001,765, filed Jun. 6, 2018, which is a continuation of International Patent Application Serial No. PCT/US2017/058591, filed Oct. 26, 2017, which claims the benefit under 35 U.S.C. 119(e) of the filing date of U.S. Provisional Application Serial No. 62/412,932, filed Oct. 26, 2016, the entire contents of each of which are incorporated herein by reference.

### REFERENCE TO A SEQUENCE LISTING SUBMITTED AS A TEXT FILE VIA EFS-WEB

The instant application contains a Sequence Listing which has been submitted in ASCII format via EFS-Web and is hereby incorporated by reference in its entirety. Said ASCII copy, created on Jun. 13, 2022, is named M137870056US02-NTJ, and is 47,929 bytes in size.

### FIELD

The present disclosure relates generally to the field of biotechnology and more specifically to the field of analytical chemistry.

### BACKGROUND

It is of great interest in the fields of therapeutics, diagnostics, reagents and for biological assays to be able to design, synthesize and deliver a nucleic acid, e.g., a ribonucleic acid (RNA) for example, a messenger RNA (mRNA) inside a cell, whether in vitro, in vivo, in situ or ex vivo, such as to effect physiologic outcomes which are beneficial to the cell, tissue or organ and ultimately to an organism. One beneficial outcome is to cause intracellular translation of the nucleic acid and production of at least one encoded peptide or polypeptide of interest. In some cases, RNA is synthesized in the laboratory in order to achieve these methods.

### SUMMARY

The validation and/or purification of synthesized RNA is important, particularly in therapeutic methods. Novel methods of identifying mRNA molecules are provided. In some aspects, methods described by the disclosure are useful for validating the production of therapeutic mRNA molecules. For example, laboratory-synthesized (e.g., by in vitro transcription) mRNA molecules encoding a protein of therapeutic relevance should be analyzed to ensure the absence of product-related impurities (e.g., less than full-length mRNAs, degradants, or read-through transcripts that are longer than the intended mRNA product), process-related impurities (e.g., nucleic acids and/or reagents carried over from synthesis reactions), or contaminants (e.g., exogenous or adventitious nucleic acids) from the mRNA molecules prior to administration to a subject.

In some aspects the invention is a method for determining the presence of an RNA in a mRNA sample, by determining a signature profile of the mRNA sample, comparing the signature profile to a known signature profile for a test mRNA, identifying the presence of an RNA in the mRNA sample based on a comparison with the known signature profile for the test mRNA. In other aspects the invention is a method for determining the presence of an RNA in a mRNA sample, by determining a signature profile of the mRNA sample, comparing the profile of the masses of the fragments generated to the predicted masses from the primary molecular sequence of the mRNA (e.g., a theoretical pattern), identifying the presence of an RNA in the mRNA sample based on the theoretical versus observed mass pattern and/or chromatographic pattern (e.g., an empirically-observed chromatographic pattern or an empirically-derived chromatographic pattern). In some embodiments the RNA is an impurity in the mRNA sample if the signature profile of the mRNA sample does not match the known signature profile for the test mRNA. In other embodiments the method has a sensitivity threshold such that an impurity of less than 1% of the sample is detected.

In other embodiments the method further involves identifying the presence of the test mRNA if the known signature profile for the test mRNA is included within the signature profile of the mRNA sample. In some embodiments the signature profile of the mRNA sample is determined by a method that includes a digestion step and a separation/detection step.

In some embodiments, the known signature profile for the test mRNA is determined by LC-MS/MS mRNA sequence mapping.

Accordingly, in other aspects the disclosure provides a method for confirming the identity of a test mRNA, the method comprising: (a) digesting a test mRNA with one or more nuclease enzymes (e.g., an endonuclease, such as an RNase enzyme, Cusativin, MazF, colicin E5, etc.) to produce a plurality of mRNA fragments; (b) physically separating the plurality of mRNA fragments; (c) assigning a signature to the test mRNA by detecting the plurality of fragments; (d) identifying the test mRNA by comparing the signature to a known mRNA signature, and (e) confirming the identity of the test mRNA if the signature of the test mRNA is the same as the known mRNA signature.

In other aspects the disclosure provides a method for confirming the identity of a test mRNA, the method comprising: (a) digesting a test mRNA with an RNase enzyme to produce a plurality of mRNA fragments; (b) physically separating the plurality of mRNA fragments; (c) determining the masses of the fragments; (d) identifying the test mRNA by comparing the signature to the predicted mass pattern (e.g., a theoretical pattern) and/or an empirically-derived chromatographic pattern, and (e) confirming the identity of the test mRNA if the observed masses and/or chromatograms.

In some embodiments, the target mRNA is an in vitro transcribed RNA (IVT mRNA). In some embodiments, the target mRNA is a therapeutic mRNA. In some embodiments, the RNase enzyme is RNase T1, a catalytic RNA (e.g., ribozyme, DNAzyme, etc.), RNase H, or Cusativin.

In some embodiments, the digesting occurs in a buffer. In some embodiments, the buffer comprises at least one component selected from the group consisting of: urea, EDTA, magnesium chloride (MgCl₂)and Tris. In some embodiments, the buffer further comprises 2′,3′-Cyclic-nucleotide 3′-phosphodiesterase (CNP) and/or Calf Intestinal Alkaline Phosphatase (CIP). In some embodiments, the digestion occurs at about 37° C.

In some embodiments, the digesting occurs in the presence of a blocking oligonucleotide. In some embodiments, a blocking oligonucleotide comprises at least one modified nucleotide. In some embodiments, the modification is selected from locked nucleic acid nucleotide (LNA), 2’OMe-modified nucleotide, and peptide nucleic acid (PNA) nucleotide. In some embodiments, the blocking oligonucleotide targets the 5’ untranslated region (5’UTR) or the 3’ untranslated region (3’UTR) of a test mRNA.

In some embodiments, the physical separation and/or the detecting is achieved by one or more methods selected from the group consisting of: gel electrophoresis, liquid chromatography, high pressure liquid chromatography (HPLC), and mass spectrometry. In some embodiments, the HPLC is HPLC-UV. In some embodiments, the mass spectrometry is Electrospray Ionization mass spectrometry (ESI-MS) or Matrix-assisted Laser Desorption/Ionization mass spectrometry (MALDI).

In some embodiments, the signature assigned to the test mRNA is an absorbance spectrum, a mass spectrum, a UV chromatogram, a total ion chromatogram, an extracted ion chromatogram, a combination of extracted ion chromatograms, or any combination of the foregoing.

In some embodiments, the signature of the test mRNA shares at least 70%, at least 80%, at least 90%, at least 95%, at least 99%, or at least 99.9% identity with the known mRNA signature.

In some embodiments, the test mRNA is removed from a population of mRNAs that will be administered as a therapeutic to a subject in need thereof.

A method for quality control of an RNA pharmaceutical composition is provided according to other aspects of the invention. The method involves digesting the RNA pharmaceutical composition with an RNase enzyme to produce a plurality of RNA fragments; physically separating the plurality of RNA fragments; generating a signature profile of the RNA pharmaceutical composition by detecting the plurality of fragments; comparing the signature profile with a known RNA signature profile, and determining the quality of the RNA based on the comparison of the signature profile with the known RNA signature profile. In some embodiments, the signature profile of the mRNA sample, is compared to the predicted masses from the primary molecular sequence of the mRNA (e.g., a theoretical pattern).

A pure mRNA sample, having a composition of an in vitro transcribed (IVT) RNA and a pharmaceutically acceptable carrier, that is preparable according to any of the methods described herein is provided in other aspects of the invention.

In other aspects of the invention a system for determining batch purity of an RNA pharmaceutical composition comprising: a computing system; at least one electronic database coupled to the computing system; at least one software routine executing on the computing system which is programmed to: (a) receive data comprising an RNA fingerprint of the RNA pharmaceutical composition; (b) analyze the data; (c) based on the analyzed data, determine batch purity of the RNA pharmaceutical composition is provided.

In some aspects, the disclosure provides an isolated nucleic acid represented by the formula from 5’ to 3’:

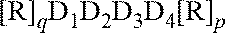

wherein each R is a modified or unmodified RNA base, D is a deoxyribonucleotide base, and each of q and p are independently an integer between 0 and 50, and wherein hybridization of the isolated nucleic acid to a mRNA in the presence of RNase H results in cleavage of the mRNA by the RNase H.

In some aspects, the disclosure provides an isolated nucleic acid represented by the formula from 5’ to 3’:

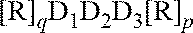

wherein each R is a modified or unmodified RNA base, D is a deoxyribonucleotide base, and each of q and p are independently an integer between 0 and 50, and wherein hybridization of the isolated nucleic acid to a mRNA in the presence of RNase H results in cleavage of the mRNA by the RNase H.

In some embodiments, at least one R is a modified RNA base, for example a 2’-O-methyl modified RNA base.

In some embodiments, each of D₁ and D₂ are unmodified deoxyribonucleotide bases. In some embodiments, D₃, D₄, or D₃ and D₄ are modified deoxyribonucleotide bases. In some embodiments, the modified deoxyribonucleotide base is 5-nitroindole or Inosine. In some embodiments, the modified deoxyribonucleotide is 4-nitroindole, 6-nitroindole, 3-nitropyrrole, a 2-6-diaminopurine, 2-amino-adenine, or 2-thio-thiamine.

In some embodiments, hybridization of the isolated nucleic acid to a mRNA in the presence of RNase H results in cleavage of the mRNA 5’ untranslated region (5’ UTR) by the RNase H. In some embodiments, cleavage of the mRNA 5’ UTR by the RNase H results in liberation of an intact mRNA Cap. In some embodiments, the isolated nucleic acid is selected from the sequences set forth in Table 5.

In some embodiments, hybridization of the isolated nucleic acid to a mRNA in the presence of RNase H results in cleavage of the mRNA 3’ untranslated region (3’ UTR) by the RNase H. In some embodiments, cleavage of the mRNA 3’ UTR by the RNase H results in liberation of an intact polyA tail. In some embodiments, the intact polyA tail further comprises at least one nucleotide of the 3’UTR of the mRNA that is not part of the polyA tail. In some embodiments, the isolated nucleic acid is selected from the sequences set forth in Table 7.

In some embodiments, hybridization of the isolated nucleic acid to a mRNA in the presence of RNase H results in cleavage of the mRNA open reading frame (ORF) by the RNase H, and no cleavage of the 5’ UTR or 3’UTR of the mRNA.

In some embodiments, mRNA digested by RNase H is in vitro transcribed (IVT) RNA. In some embodiments, mRNA digested by RNase H is a therapeutic mRNA.

In some aspects, the disclosure provides a composition comprising a plurality of isolated nucleic acids as described by the disclosure. In some embodiments, the plurality is three or more isolated nucleic acids.

In some embodiments, the plurality comprises: (i) at least one isolated nucleic acid that results in cleavage of the mRNA 5’UTR, (ii) at least one isolated nucleic acid that results in cleavage of the mRNA 3’UTR; and, (iii) at least one isolated nucleic acid that results in cleavage of the mRNA ORF. In some embodiments, the plurality comprises between 1 and 100 isolated nucleic acids that each results in cleavage of the mRNA 5’UTR.

In some embodiments, the plurality comprises between 5 and 50 isolated nucleic acids that each results in cleavage of the mRNA 5’UTR. In some embodiments, the plurality comprises between 10 and 20 isolated nucleic acids that each results in cleavage of the mRNA 5’UTR. In some embodiments, the plurality comprises between 1 and 5 isolated nucleic acids that each results in cleavage of the mRNA 5’UTR.

In some embodiments, the plurality comprises between 5 and 50 isolated nucleic acids that each results in cleavage of the mRNA 3’UTR. In some embodiments, the plurality comprises between 10 and 20 isolated nucleic acids that each results in cleavage of the mRNA 3’UTR. In some embodiments, the plurality comprises between 1 and 5 isolated nucleic acids that each results in cleavage of the mRNA 3’UTR.

In some embodiments, the plurality comprises between 5 and 50 isolated nucleic acids that each results in cleavage of the mRNA ORF. In some embodiments, the plurality comprises between 10 and 20 isolated nucleic acids that each results in cleavage of the mRNA ORF. In some embodiments, the plurality comprises between 1 and 5 isolated nucleic acids that each results in cleavage of the mRNA ORF.

In some embodiments, compositions described by the disclosure further comprise a buffer, and optionally, RNase H enzyme.

In some aspects, the disclosure provides a method for quality control of an RNA pharmaceutical composition, comprising: digesting the RNA pharmaceutical composition with an RNase H enzyme to produce a plurality of RNA fragments; physically separating the plurality of RNA fragments; generating a signature profile of the RNA pharmaceutical composition by detecting the plurality of fragments; comparing the signature profile with a known RNA signature profile, and determining the quality of the RNA based on the comparison of the signature profile with the known RNA signature profile.

In some embodiments, the digesting step comprises contacting the RNA pharmaceutical composition with an RNase enzyme (e.g., RNase H) and, optionally, one or more isolated nucleic acids as described by the disclosure, or a pharmaceutical composition as described by the disclosure, prior to contacting the RNA pharmaceutical composition with the RNase enzyme. In some embodiments, the digesting step is performed in the presence of one or more blocking oligonucleotides.

In some aspects, the disclosure provides a method for characterizing a mRNA, comprising: contacting an mRNA with an RNase H enzyme, and optionally, an isolated nucleic acid as described by the disclosure; physically separating a cleaved 3’ untranslated region (3’ UTR) from the mRNA; generating a signature profile of the mRNA by detecting the cleaved mRNA 3’ UTR; comparing the signature profile with a known RNA signature profile, and, quantifying the polyA tail length of the mRNA based upon the comparison of the signature profile with the known RNA signature profile. In some embodiments, the digesting step is performed in the presence of one or more blocking oligonucleotides.

In some aspects, the disclosure provides a method for characterizing a mRNA, comprising: contacting an mRNA with an RNase H enzyme, and optionally, an isolated nucleic acid as described by the disclosure; physically separating a cleaved 5’ untranslated region (5’ UTR) from the mRNA; generating a signature profile of the mRNA by detecting the cleaved mRNA 5’ UTR; comparing the signature profile with a known RNA signature profile, and, determining the Cap structure of the mRNA based upon the comparison of the signature profile with the known RNA signature profile. In some embodiments, the digesting step is performed in the presence of one or more blocking oligonucleotides.

In some aspects, the disclosure provides a method for identifying an RNA pharmaceutical composition having a desired structure, comprising: digesting the RNA pharmaceutical composition with an RNase H enzyme to produce a plurality of RNA fragments; physically separating the plurality of RNA fragments; generating a signature profile of the RNA pharmaceutical composition by detecting the plurality of fragments; comparing the signature profile with a known RNA signature profile, and determining the quality of the RNA based on the comparison of the signature profile with the known RNA signature profile.

In some embodiments, the step of generating a signature profile comprises identifying the 5’UTR (e.g., 5’ cap) structure of the RNA, poly(A) tail length of the RNA, or the 5’UTR structure and poly(A) tail length of the RNA in the RNA pharmaceutical composition. In some embodiments, the method further comprises identifying the RNA pharmaceutical composition as suitable for therapeutic use (e.g., use in a human subject) based on the quality of the RNA.

Without wishing to be bound by any particular theory, methods of identifying an RNA pharmaceutical composition having a desired structure described by the disclosure may be useful, in some embodiments, as a “release assay” which determines whether a particular batch of a manufactured mRNA therapeutic is acceptable (e.g., has an acceptable safety profile, purity, activity, etc.) for therapeutic use in a particular population, such as human subjects (e.g., release into the marketplace).

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF THE DRAWINGS

FIG. 1 shows the total number of RNA fragments predicted to be generated by RNase T1 digestion of mRNA Sample 1. For example, there are 92 2-mer fragments generated by this digestion.

FIG. 2 shows the number of unique fragments predicted to be generated by RNase T1 digestion of mRNA Sample 1. For example, there are 31 unique 6-mer fragments generated by this RNase digestion.

FIG. 3 shows the mass of different fragment lengths predicted to be generated. For example, 10% of the total mass of mRNA sample 1 is digested into 6-mers.

FIG. 4 shows analyses of Sample 1 after RNase T1 digestion by HPLC produces a chromatographic pattern that represents a unique fingerprint for Sample 1.

FIG. 5 shows representative HPLC data demonstrating the reproducibility of RNase digestion. Two samples of mRNA Sample 1 were digested and run on an HPLC column. The trace patterns for each digestion of mRNA Sample 1 (e.g., Run 1 and Run 2) demonstrate good peak alignments.

FIG. 6 shows representative HPLC data demonstrating the unique pattern generated by RNase digestion of two different mRNA samples (e.g., mRNA Sample 1 and mRNA Sample 2) demonstrating poor peak alignments, thereby enabling differentiation of these two samples.

FIG. 7 shows representative HPLC data demonstrating the reproducibility of RNase digestion across multiple digests. Separate aliquots of mRNA Sample 3 were RNase digested (Digest 1, 2 and 3) and run on an HPLC column. The trace patterns for each digestion demonstrate good peak alignments.

FIG. 8 shows representative HPLC data illustrating that digestion with different RNase enzymes (e.g., RNase T1 or RNase A) leads to the generation of distinct trace patterns. Digestion of mRNA Sample 3 with RNase T1 provides a trace pattern exhibiting greater complexity than digestion with RNase A.

FIG. 9 shows representative ESI-MS data. Two mRNA samples (mRNA Sample 1 and mRNA Sample 2) were digested with RNase T1. ESI-MS was performed on digested samples. Results demonstrate that unique mass traces are generated for each sample.

FIGS. 10A-10B show representative data from ESI-MS of two RNase T1-digested mRNA samples (mRNA Sample 4 and mRNA Sample 5). Data demonstrates that each mass fingerprint is unique.

FIG. 11 shows representative data from LC/MS of RNase T1-digested mRNA encoding mCherry.

FIG. 12 shows a schematic of one embodiment of mRNA Cap structure.

FIG. 13 shows structures of partial mRNA Cap synthesis.

FIG. 14 shows representative data of mRNA tail length determination by reversed-phase ion paired chromatography (RP-IP) with UV detection. Data indicate that length determination by relative retention time is not robust across different mRNA constructs. Data indicate that it is difficult to measure polyA tail length without cleaving it from the mRNA molecule.

FIG. 15 shows a comparison of robustness and specificity for mRNA digestion using DNAzyme, RNase H, RNase T1, and RNase A.

FIG. 16 shows a schematic depiction of mRNA Cap fragment liberation by DNAzyme.

FIG. 17 shows representative data of MS analysis of mRNA Cap after sequence-specific DNAzyme digestion.

FIG. 18 shows representative MS data of a one-pot specific cap/tail cleavage of mRNA using DNAzyme. Data indicate that undigested mRNA and tail species co-elute due to the hydrophobicity of the polyA tail.

FIG. 19 shows representative MS data of a one-pot specific cap/tail cleavage of mRNA using DNAzyme. Data indicate that undigested mRNA and tail species co-elute due to the hydrophobicity of the polyA tail.

FIG. 20 shows RNase H guide strand design for digestion of mRNA Cap sequence.

FIG. 21 shows representative data of an extracted ion chromatogram (EIC) corresponding to nucleotide length of a mRNA fragment obtained by digesting with RNase H directed by guide strands of uniform length having modified DNA positions. Specific cleavage is observed with a single 2’-O-methyl RNA flanking the final DNA base designating the cut site and having a total guide strand length of 9 nucleobases, as indicated by the peak labeled “8 nt”.

FIG. 22 shows representative data of area versus fragment length (nt) and RNA base cleaved of a mRNA fragment obtained by digesting with RNase H directed by guide strands of uniform length having modified DNA positions. Reducing guide strand length from 16 nt (“8_AA”) to 9 nt (“L9 8 nt”) does not impact the signal of the resulting target fragment as measured by MS.

FIG. 23 shows representative MS data comparing mRNA Cap digestion by DNAzyme (top) and RNase H (bottom). For some constructs, DNAzyme does not cleave the 5’UTR efficiently, or at all, whereas RNase H does cleave the 5’UTR efficiently.

FIG. 24 shows representative data of RNase H cleavage of mRNA tail (e.g., polyA tail). Undigested mRNA and tail species co-elute due to the hydrophobicity of the polyA tail.

FIG. 25 shows representative data of ESI total ion current chromatogram (ESI-TIC) for RNase H digests of human erythropoietin (hEpo) mRNA tail variants. Data indicate that undigested mRNA-Tail and/or cleaved mRNA co-elute with the target Poly A species. Data also indicate co-elution of RNase H guide strand with targeted tail species that fall between lengths of 0 (“T0”) and 60 nucleotides (“T60”).

FIG. 26 shows representative data relating to the sequence-specificity of RNase T1 mRNA fingerprinting. Chromatograms for three different mRNA: “mRNA A” produced from plasmid DNA, “mRNA A” produced from rolling circle amplification (RCA)-amplified DNA, and “mRNA B” produced from RCA-amplified DNA were overlaid and chromatographic fingerprints were compared.

FIG. 27 shows a schematic depiction of one embodiment of mRNA Cap digestion by RNase T1.

FIG. 28 shows representative LC and MS data related to mRNA Cap digestion using RNase T1. Data indicate that RNase T1 digestion allows quantitation of four Cap subspecies but not Uncapped mRNA.

FIG. 29 shows representative data related to the limit of detection (LOD) of mRNA tail variants by RNase T1 digestion.

FIG. 30 shows a schematic describing design of RNase H guide strands targeting the open reading frame (ORF) of mRNA.

FIG. 31 shows representative data illustrating the impact of RNase H guide strand length and 3’ modification on target tail fragment identification by liquid chromatography (LC) UV detection and LC-MS detection.

FIG. 32 shows representative data illustrating the impact of RNase H guide strand length and 3’ modification on target tail fragment identification by MS.

FIG. 33 shows representative data illustrating the impact of RNase H guide strand length and 3’ modification on mRNA tail length quantitation as measured by MS. Data are shown for digestions directed by four Guide Strand #4 variants.

FIG. 34 shows representative data illustrating the impact of RNase H guide strand modification on mRNA tail length quantitation as measured by MS. Guide strands were modified by substitution of non-traditional nucleobases (5-nitroindole “N”, and Inosine “I”) at a site within the DNA/RNA recognition motif of the guide stand. Data indicate that nucleotides at positions d3 and d4 of the DNA/RNA recognition motif are not required to be traditional nucleobases and can be unconventional, as cleavage of target tail fragment is observed. RNase H cleavage is not observed when positions d1 and d2 of the DNA/RNA recognition motif are non-traditional nucleobases.

FIG. 35 shows representative data illustrating the impact of RNase H guide strand modification on mRNA tail length quantitation as measured by MS. Guide strands were modified by substitution of non-traditional nucleobases (5-nitroindole “N”, and Inosine “I”) at positions m5 and m6 of the guide stand. Data indicate cleavage does not occur when positions m5 or m6 are not a traditional 2’-deoxyribonucleotide.

FIGS. 36A-36C show representative data illustrating RNase H guide strand modification on Epo mRNA tail length quantitation as measured by MS. The Epo mRNA digested has a tail length of 95 nucleotides (T95 (SEQ ID NO: 45)). FIG. 36A shows digestion of Epo T95 with RNase H Guide strand # 4 and a Guide strand #4 variant, which contains a 3’ 6-carboxyfluoroscein (3’-6FAM) modification. FIG. 36B shows Guide strand #4 variants, which contain a 5-nitorindole modification at position d3 (top) or d4 (bottom). FIG. 36C shows Guide strand #4 variants, which contain an Inosine modification at position d3 (top) or d4 (bottom).

FIG. 37 shows a schematic depicting the mRNA digest protocol used in this example. Briefly, RNase H guide strands specific for Cap and Tail regions, but not specific for open reading frame (e.g., “coding region”) are used to digest an mRNA. LC-MS analysis is then performed and the following data are analyzed: (i) Cap identification and relative quantification; (ii) polyA tail length identification and relative quantification; optionally, (iii) total digest and mapping.

FIG. 38 shows representative data of mRNA Cap and tail one pot digestion using RNase H. The top panel of FIG. 38 shows analysis of combined Cap/tail digestion by total ion current chromatogram (TIC) and the bottom panel of FIG. 38 shows the same combined Cap/tail digest analyzed by UV detection.

FIG. 39 shows representative quality control data for a combined Cap/tail one pot digestion. The top panel of FIG. 39 shows analysis by TIC and the bottom panel shows analysis by UV detection.

FIG. 40 shows representative data for the analysis of Cap region of interest as identified by TIC. A single peak corresponding to Cap1 (e.g., complete 5’ Cap) was identified.

FIG. 41 shows representative data for the analysis of tail region of interest as identified by TIC.

FIGS. 42A-42B show representative data related to Poly(A) tail assay development. FIG. 42A shows representative LC-MS data of hEPO (theoretical tail length of A95 (SEQ ID NO: 45)) interrogating RNase H activity with four different tail guides. Tail guides were designed to target the 3’UTR, allowing for tailless and Aₙ tail lengths to be identified. FIG. 42B shows representative LC profile (TIC) generated for hEPO with different theoretical tail lengths. Overlays of RNase H digestion products for tail lengths of A₀ (tailless), A₆₀ (SEQ ID NO: 46), A₉₅ (SEQ ID NO: 45) and A₁₄₀ (SEQ ID NO: 47) are shown.

FIGS. 43A-43B show representative data related to evaluation the impact of mRNA tail length on MS signal. FIG. 43A demonstrates the relationship between MS signal and molar input of mRNA obtained for four different tail lengths (A₉₅ (SEQ ID NO: 45), A₆₀ (SEQ ID NO: 46), A₄₀ (SEQ ID NO: 113), A₀). FIG. 43B shows the linear relationship between total MS signal and molar input of each tail variant.

FIG. 44 shows representative data for a total ion chromatogram (TIC) of a one-pot cap/tail RNase H assay. The box on the left side of the histogram highlights the retention time region of interests for the cap variants, while the box on the right side of the histogram indicates the major region of interest for the tail analysis. Not shown in the target region where tailless elutes (3.0-3.2 mins).

FIGS. 45A-45B show representative data for a one-pot processed cap and tail variants. FIG. 45 A shows representative data for an extracted ion chromatogram (EIC) for the target cap variants. In this sample, only Cap 1 was identified. FIG. 45B shows representative deconvoluted MS data of the one-pot cap/tail RNase H assay for determining Poly (A) tail length. The different tail lengths are shown. This mRNA has a tail variants ranging from A₉₄-A₁₀₀ in length (SEQ ID NO: 114).

FIGS. 46A-46C show representative date for the interrogation of substrate dependent RNase H activity via cap assay. FIG. 46A shows cleavage efficiency of RNase H relative to RNA bases 5’ and 3’ of the cut site was evaluated. Data indicate that RNase H prefers to cut after A, and before A or G. In some embodiments, Uridine, modified in this case, prevents cleavage 3’ of the cut site, but only inhibits 5’ of the cut site. FIG. 46B shows an alignment of a 5’ UTR (comprising a cap) with a shortened 13-nucleotide version and the most efficient guide strand identified in this example. Data indicates that 2’OMe bases mismatched to the 3’ of the cut site do not have an effect on cleavage. FIG. 46C shows that RNase H guides show efficacy with 3’ mismatches and there is no evidence that nearest neighbors to the cut site play a role in determining cleavage efficiency.

FIG. 47 is a schematic depiction of a strategy for RNase blocking using complementary oligonucleotides. Briefly, complementary oligonucleotides bind to a target mRNA and block the activity of RNase (e.g., RNase T1) and other nucleases capable of cutting dsRNA.

FIG. 48 shows examples of modified nucleic acids, such as locked nucleic acids (LNAs), 2’-O-methyl-modified (2’OMe) nucleic acids, and peptide nucleic acids (PNAs), that increase binding affinity of oligonucleotides (e.g., blocking oligonucleotides) to mRNA.

FIG. 49 shows representative data for RNase T1 blocking efficiency by modified nucleic acid (LNA, PNA, 2’OMe) blocking oligos as measured by LC/MS.

FIG. 50 shows representative data for RNase T1 blocking efficiency at different concentrations of RNase T1 by modified nucleic acid (LNA, PNA, 2’OMe) blocking oligos as measured by LC/MS.

FIG. 51 shows one example of a workflow for mRNA sequence mapping by LC-MS.

FIG. 52 shows examples of test mRNA digestion using RNase T1 (which cleaves RNA after each G) in parallel with Cusativin (which cleaves RNA after poly-C).

FIG. 53 shows examples MS/MS isomeric differentiation by oligo fragmentation pattern comparison.

FIG. 54 shows an example of a graphic user interface (GUI) for mRNA LC-MS/MS search engine with mRNA in silico digestion, LC-MS/MS database generation and search, and oligo identification.

FIG. 55 shows an example of sequence mapping output, and performance evaluation with different MS gathering mode and enzyme(s) for digestion.

### DETAILED DESCRIPTION

Delivery of mRNA molecules to a subject in a therapeutic context is promising because it enables intracellular translation of the mRNA and production of at least one encoded peptide or polypeptide of interest without the need for nucleic acid-based delivery systems (e.g., viral vectors and DNA-based plasmids). Therapeutic mRNA molecules are generally synthesized in a laboratory (e.g., by in vitro transcription). However, there is a potential risk of carrying over impurities or contaminants, such as incorrectly synthesized mRNA and/or undesirable synthesis reagents, into the final therapeutic preparation during the production process. In order to prevent the administration of impure or contaminated mRNA, the mRNA molecules can be subject to a quality control (QC) procedure (e.g., validated or identified) prior to use. Validation confirms that the correct mRNA molecule has been synthesized and is pure.

Typical assays for examining the purity of an RNA sample do not achieve the level of accuracy that can be achieved by the direct structural characterization involving RNA fingerprinting of the instant methods. According to some aspects of the invention a method of analyzing and characterizing an RNA sample is provided. The method involves determining a signature profile of the mRNA sample, comparing the signature profile to a known signature profile for a test mRNA, identifying the presence of an RNA in the mRNA sample based on a comparison with the known signature profile for the test mRNA.

In other aspects the invention is a method for determining the presence of an RNA in a mRNA sample, by determining a signature profile of the mRNA sample, comparing the profile of the masses and/or retention times of the fragments generated to the expected masses and/or retention times from the primary molecular sequence of the RNA (e.g., a theoretical pattern), identifying the presence of an RNA in the mRNA sample based on the theoretical versus observed mass pattern and/or chromatographic pattern.

The methods of the invention can be used for a variety of purposes where the ability to identify and RNA fingerprint is important. For instance, the methods of the invention are useful for monitoring batch-to-batch variability of an RNA composition or sample. The purity of each batch may be determined by determining any differences in the signature profile in comparison to a known signature profile or a theoretical profile of predicted masses from the primary molecular sequence of the RNA. These signatures are also useful for monitoring the presence of unwanted nucleic acids which may be active components in the sample. The methods may also be performed on at least two samples to determine which sample has better purity or to otherwise compare the purity of the samples.

Thus, in some instances the methods of the invention are used to determine the purity of an RNA sample. The term “pure” as used herein refers to material that has only the target nucleic acid active agents such that the presence of unrelated nucleic acids is reduced or eliminated, i.e., impurities or contaminants, including RNA fragments. For example, a purified RNA sample includes one or more target or test nucleic acids but is preferably substantially free of other nucleic acids. As used herein, the term “substantially free” is used operationally, in the context of analytical testing of the material. Preferably, purified material substantially free of impurities or contaminants is at least 95% pure; more preferably, at least 98% pure, and more preferably still at least 99% pure. In some embodiments a pure RNA sample is comprised of 100% of the target or test RNAs and includes no other RNA. In some embodiments it only includes a single type of target or test RNA.

A “polynucleotide” or “nucleic acid” is at least two nucleotides covalently linked together, and in some instances, may contain phosphodiester bonds (e.g., a phosphodiester “backbone”) or modified bonds, such as phosphorothioate bonds. An “engineered nucleic acid” is a nucleic acid that does not occur in nature. In some instances the RNA in the RNA sample is an engineered RNA sample. It should be understood, however, that while an engineered nucleic acid as a whole is not naturally-occurring, it may include nucleotide sequences that occur in nature. Thus, a “polynucleotide” or “nucleic acid” sequence is a series of nucleotide bases (also called “nucleotides”), generally in DNA and RNA, and means any chain of two or more nucleotides. The terms include genomic DNA, cDNA, RNA, any synthetic and genetically manipulated polynucleotide,. This includes single- and double-stranded molecules; i.e., DNA-DNA, DNA-RNA, and RNA-RNA hybrids as well as “protein nucleic acids” (PNA) formed by conjugating bases to an amino acid backbone.

The methods of the invention involve the analysis of RNA samples. An RNA in an RNA sample typically is composed of repeating ribonucleosides. It is possible that the RNA includes one or more deoxyribonucleosides. In preferred embodiments the RNA is comprised of greater than 60%, 70%, 80% or 90% of ribonucleosides. In other embodiments the RNA is 100% comprised of ribonucleosides. The RNA in an RNA sample is preferably an mRNA.

As used herein, the term “messenger RNA (mRNA)” refers to a ribonucleic acid that has been transcribed from a DNA sequence by an RNA polymerase enzyme, and interacts with a ribosome to synthesize protein encoded by DNA. Generally, mRNA are classified into two sub-classes: pre-mRNA and mature mRNA. Precursor mRNA (pre-mRNA) is mRNA that has been transcribed by RNA polymerase but has not undergone any post-transcriptional processing (e.g., 5’capping, splicing, editing, and polyadenylation). Mature mRNA has been modified via post-transcriptional processing (e.g., spliced to remove introns and polyadenylated region) and is capable of interacting with ribosomes to perform protein synthesis.

mRNA can be isolated from tissues or cells by a variety of methods. For example, a total RNA extraction can be performed on cells or a cell lysate and the resulting extracted total RNA can be purified (e.g., on a column comprising oligo-dT beads) to obtain extracted mRNA.

Alternatively, mRNA can be synthesized in a cell-free environment, for example by in vitro transcription (IVT). IVT is a process that permits template-directed synthesis of ribonucleic acid (RNA) (e.g., messenger RNA (mRNA)). It is based, generally, on the engineering of a template that includes a bacteriophage promoter sequence upstream of the sequence of interest, followed by transcription using a corresponding RNA polymerase. In vitro mRNA transcripts, for example, may be used as therapeutics in vivo to direct ribosomes to express protein therapeutics within targeted tissues.

Traditionally, the basic components of an mRNA molecule include at least a coding region, a 5’UTR, a 3’UTR, a 5’ cap and a poly-A tail. IVT mRNA may function as mRNA but are distinguished from wild-type mRNA in their functional and/or structural design features which serve to overcome existing problems of effective polypeptide production using nucleic-acid based therapeutics. For example, IVT mRNA may be structurally modified or chemically modified. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. For example, the polynucleotide “ATCG” may be chemically modified to “AT-5meC-G”. The same polynucleotide may be structurally modified from “ATCG” to “ATCCCG”. Here, the dinucleotide “CC” has been inserted, resulting in a structural modification to the polynucleotide.

An RNA may comprise naturally occurring nucleotides and/or non-naturally occurring nucleotides such as modified nucleotides. In some embodiments, the RNA polynucleotide of the RNA vaccine includes at least one chemical modification. In some embodiments, the chemical modification is selected from the group consisting of pseudouridine, N1-methylpseudouridine, 2-thiouridine, 4’-thiouridine, 5-methylcytosine, 2-thio-1-methyl-1-deaza-pseudouridine, 2-thio-1-methyl-pseudouridine, 2-thio-5-aza-uridine , 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2’-O-methyl uridine. Other exemplary chemical modifications useful in the mRNA described herein include those listed in U.S. Published Pat. Application 2015/0064235.

In some embodiments the methods may be used to detect differences in chemical modification of an mRNA sample. The presence of different chemical modifications patterns may be detected using the methods described herein.

An “in vitro transcription template (IVT),” as used herein, refers to deoxyribonucleic acid (DNA) suitable for use in an IVT reaction for the production of messenger RNA (mRNA). In some embodiments, an IVT template encodes a 5’ untranslated region, contains an open reading frame, and encodes a 3’ untranslated region and a polyA tail. The particular nucleotide sequence composition and length of an IVT template will depend on the mRNA of interest encoded by the template.

A “5’ untranslated region (UTR)” refers to a region of an mRNA that is directly upstream (i.e., 5’) from the start codon (i.e., the first codon of an mRNA transcript translated by a ribosome) that does not encode a protein or peptide.

A “3’ untranslated region (UTR)” refers to a region of an mRNA that is directly downstream (i.e., 3’) from the stop codon (i.e., the codon of an mRNA transcript that signals a termination of translation) that does not encode a protein or peptide.

An “open reading frame” is a continuous stretch of DNA beginning with a start codon (e.g., methionine (ATG)), and ending with a stop codon (e.g., TAA, TAG or TGA) and encodes a protein or peptide.

A “polyA tail” is a region of mRNA that is downstream, e.g., directly downstream (i.e., 3’), from the 3’ UTR that contains multiple, consecutive adenosine monophosphates. A polyA tail may contain 10 to 300 (SEQ ID NO: 116) adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 (SEQ ID NO: 116) adenosine monophosphates. In some embodiments, a polyA tail contains 50 to 250 (SEQ ID NO: 117) adenosine monophosphates. In a relevant biological setting (e.g., in cells, in vivo, etc.) the poly(A) tail functions to protect mRNA from enzymatic degradation, e.g., in the cytoplasm, and aids in transcription termination, export of the mRNA from the nucleus, and translation. However, in some embodiments, mRNA molecules do not comprise a polyA tail. In some embodiments, such molecules are referred to as “tailless”.

In some embodiments, the test or target mRNA (e.g., IVT mRNA) is a therapeutic mRNA. As used herein, the term “therapeutic mRNA” refers to an mRNA molecule (e.g., an IVT mRNA) that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment of the following diseases and conditions: bacterial infections, viral infections, parasitic infections, cell proliferation disorders, genetic disorders, and autoimmune disorders.

A “test mRNA” or “target mRNA” (used interchangeably herein) is an mRNA of interest, having a known nucleic acid sequence. The test mRNA may be found in a RNA or mRNA sample. In addition to the test mRNA the RNA or mRNA sample may include a plurality of mRNA molecules or other impurities obtained from a larger population of mRNA molecules. For example, after the production of IVT mRNA, a test mRNA sample may be removed from the population of IVT mRNA in order to assay for the purity and/or to confirm the identity of the mRNA produced by IVT.

In some embodiments, the test mRNA is assigned a signature, referred to as a signature profile for a test mRNA. As used herein, the term “signature” refers to a unique identifier or fingerprint that uniquely identifies an mRNA. A “signature profile for a test mRNA” is a signature generated from an mRNA sample suspected of having a test mRNA based on fragments generated by digestion with a particular RNase enzyme. For example, digestion of an mRNA with RNase T1 and subsequent analysis of the resulting plurality of mRNA fragments by HPLC or mass spec produces a trace or mass profile, or signature that can only be created by digestion of that particular mRNA with RNase T1.

In other embodiments, test mRNA is digested with RNase H. RNase H cleaves the 3’-O-P bond of RNA in a DNA/RNA duplex substrate to produce 3’-hydroxyl and 5‘-phosphate terminated products. Therefore, specific nucleic acid (e.g., DNA, RNA, or a combination of DNA and RNA) oligos can be designed to anneal to the test mRNA, and the resulting duplexes digested with RNase H to generate a unique fragment pattern (resulting in a unique mass profile) for a given test mRNA.

In some aspects, the disclosure provides isolated nucleic acids (e.g., specific oligos) that anneal to a mRNA (e.g., a test mRNA) and direct RNase H cleavage of the mRNA. In some embodiments, the isolated nucleic acids are referred to as “guide strands”. The disclosure relates, in part, to the discovery that an isolated nucleic acid represented by the formula from 5’ to 3’:

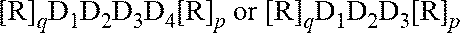

wherein each R is an unmodified or modified RNA base, D is a deoxyribonucleotide base, and each of q and p are independently an integer between 0 and 15, hybridize in a sequence-specific manner to a mRNA in the presence of RNase H and direct cleavage of the mRNA by the RNase H.

In some embodiments, at least one R is a modified RNA base, for example a 2’-O-methyl modified RNA base.

The length of each of [R]_{q} and [R]ₚ can independently vary in length. For example, in some embodiments, q is an integer between 0 and 50 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) and p is an integer between 0 and 50 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50).

In some embodiments, q is an integer between 0 and 30 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, or 30) and p is an integer between 0 and 50 (e.g., 0, 1, 2, 3, 4, 5, 6, 7, 8, 9, 10, 11 ,12, 13, 14, 15, 16, 17, 18, 19, 20, 21, 22, 23, 24, 25, 26, 27, 28, 29, or 30).

In some embodiments, q is an integer between 0 and 15 (e.g., 0, 1, 2, 3, 4, 5, 6, 7, 8, 9, 10, 11 ,12, 13, 14, or 15) and p is an integer between 0 and 15 (e.g., 0, 1, 2, 3, 4, 5, 6, 7, 8, 9, 10, 11 ,12, 13, 14, or 15).

In some embodiments, q is an integer between 0 and 6 (e.g., 0, 1, 2, 3, 4, 5, or 6) and p is an integer between 1 and 10 (e.g., 1, 2, 3, 4, 5, 6, 7, 8, 9, or 10). In some embodiments, p is an integer between 0 and 6 (e.g., 0, 1, 2, 3, 4, 5, or 6) and q is an integer between 1 and 10 (e.g., 1, 2, 3, 4, 5, 6, 7, 8, 9, or 10).

In some embodiments, each of D₁ and D₂ are unmodified (e.g., natural) deoxyribonucleotide bases. As used herein, “unmodified deoxyribonucleotide base” refers to a natural DNA base, such as adenosine, guanosine, cytosine, thymine, or uracil. In some embodiments, D₃, D₄, or D₃ and D₄ are unnatural (e.g., modified) deoxyribonucleotide bases. The term “modified deoxyribonucleotide base,” “nucleotide analog,” or “altered nucleotide” refers to a non-standard nucleotide, including non-naturally occurring deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310.

Nucleotide analogs may also comprise modifications to the sugar portion of the nucleotides. For example the 2’ OH-group may be replaced by a group selected from H, OR, R, F, Cl, Br, I, SH, SR, NH₂, NHR, NR₂, COOR, or, wherein R is substituted or unsubstituted C₁-C.₆ alkyl, alkenyl, alkynyl, aryl, etc.

In some embodiments, the unnatural (e.g., modified) deoxyribonucleotide base is 5-nitroindole or Inosine. In some embodiments, the modified deoxyribonucleotide is 4-nitroindole, 6-nitroindole, 3-nitropyrrole, a 2-6-diaminopurine, 2-amino-adenine, or 2-thio-thiamine.

In some aspects, the disclosure relates to the discovery that hybridization of certain isolated nucleic acids (e.g., guide strands) to a mRNA in the presence of RNase H results in specific separation of mRNA 5’ untranslated region (5’ UTR) from the mRNA by the RNase H. Without wishing to be bound by any particular theory, separation of intact 5’UTR of an mRNA allows for characterization of the 5’ cap structure of the mRNA, for example by mass spectrometric analysis of the 5′ cap fragment. In some embodiments, isolated nucleic acids direct separation of intact 5’UTR of mRNA without digestion of other regions of the mRNA (e.g., open reading frame (ORF), 3’ untranslated region (UTR), polyA tail, etc.).

Isolated nucleic acids (e.g., guide strands) that direct in RNase H cleavage of mRNA 5′ UTR can hybridize anywhere within the 5′ UTR region (e.g. the region directly upstream of the first nucleotide of the mRNA initiation codon) of an mRNA. For example, in some embodiments, an isolated nucleic acid (e.g., guide strand) hybridizes to a mRNA 5’ UTR between 1 nucleotide and about 200 nucleotides upstream of the first nucleotide of the initiation codon. In some embodiments, an isolated nucleic acid (e.g., guide strand) hybridizes to a mRNA 5’ UTR between 1 nucleotide and about 100 nucleotides upstream of the first nucleotide of the initiation codon. In some embodiments, an isolated nucleic acid (e.g., guide strand) hybridizes to a mRNA 5’ UTR between 1 nucleotide and about 50 nucleotides (e.g., 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides) upstream of the first nucleotide of the initiation codon. Non-limiting examples of isolated nucleic acids (e.g., guide strands) that result in RNase H cleavage of mRNA 5’UTR are shown in Table 6.

In some aspects, the disclosure relates to the discovery that hybridization of certain isolated nucleic acids (e.g., guide strands) to a mRNA in the presence of RNase H results in specific separation of mRNA 3’ untranslated region (3’ UTR) from the mRNA by the RNase H. Without wishing to be bound by any particular theory, separation of intact 3’UTR of an mRNA allows for characterization of the 3’ polyA tail of the mRNA, for example by mass spectrometric analysis. In some embodiments, isolated nucleic acids direct separation of intact 3’UTR of mRNA without digestion of other regions of the mRNA (e.g., open reading frame (ORF), 5’ UTR, etc.).

Isolated nucleic acids (e.g., guide strands) that result in RNase H cleavage of mRNA 3’ UTR can hybridize anywhere within the 3’ UTR region (e.g. the region directly downstream of the last nucleotide of the mRNA stop codon) of an mRNA. For example, in some embodiments, an isolated nucleic acid (e.g., guide strand) hybridizes to a mRNA 3’ UTR between 1 nucleotide and about 200 nucleotides downstream of the last nucleotide of the stop codon. In some embodiments, an isolated nucleic acid (e.g., guide strand) hybridizes to a mRNA 3’ UTR between 1 nucleotide and about 100 nucleotides downstream of the last nucleotide of the stop codon. In some embodiments, an isolated nucleic acid (e.g., guide strand) hybridizes to a mRNA 3’ UTR between 1 nucleotide and about 50 nucleotides (e.g., 1, 2, 3, 4, 5, 6, 7, 8, 9, 10, 11 ,12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides) downstream of the last nucleotide of the stop codon. In some embodiments, the isolated nucleic acid is selected from the sequences set forth in Table 8.

In some embodiments, hybridization of the isolated nucleic acid to a mRNA in the presence of RNase H results in cleavage of the mRNA open reading frame (ORF) by the RNase H, and no cleavage of the 5’ UTR or 3’UTR of the mRNA. Without wishing to be bound by any particular theory, shortening the length of an isolated nucleic acid (e.g. guide strand) allows it to land in more places on the ORF, progressively reducing secondary structure leading to specific total digest of the mRNA. Accordingly, in some embodiments, an isolated nucleic acid (e.g., guide strand) that directs cleavage of a mRNA ORF is between 4 and 16 nucleotides in length (e.g., 4, 5, 6, 7, 8, 9, 10, 11, 12, 13, 14, 15, or 16 nucleotides in length). In some embodiments, a guide strand comprises a single 5’ or 3’ positioned 2’O-methyl RNA and four unmodified DNA bases. In some embodiments, a guide strand consists of four unmodified DNA bases.

In some aspects, the disclosure relates to the discovery that the fragmentation repertoire (e.g., number of possible fragments produced by RNase digestion) of an mRNA molecule may be increased by including blocking oligonucleotides (also referred to as “blocking oligos”) during RNase digestion. As used herein, a “blocking oligo” refers to an oligonucleotide (e.g., polynucleotide) that hybridizes or binds to a test mRNA and thus inhibits cleavage of the mRNA at the location of the hybridization. Generally, a blocking oligo may be between about 2 and about 100 nucleotides in length (e.g., any integer between 2 and 100, inclusive), for example, about 5, 10, 15, 20, 25, 30, 40, 50, 75, or 100 nucleotides in length. A blocking oligo may comprise ribonucleotide bases, deoxyribonucleotide bases, unnatural nucleobases, or any combination thereof. In some embodiments, a blocking oligo comprises one or more modified nucleic acid bases. Examples of modified nucleic acid bases include but are not limited to locked nucleic acid (LNA) bases, 2’O-methyl (2’OMe)-modified bases, and peptide nucleic acids (PNAs). Without wishing to be bound by any particular theory, blocking oligos comprising one or more modified nucleic acid bases increase binding affinity between the blocking oligo and the test mRNA.

In some embodiments, a blocking oligo binds to (e.g., hybridizes with) an untranslated portion of a test mRNA, for example a 5’ untranslated region (5’UTR) or a 3’ untranslated region (3’UTR). In some embodiments, a blocking oligo binds to (e.g., hybridizes with) a protein coding region of a test mRNA.

Compositions comprising a plurality of isolated nucleic acids (e.g., a cocktail of guide strands) are also contemplated by the disclosure. In some embodiments, compositions comprising a plurality of isolated nucleic acids (e.g., a cocktail of guide strands) are useful for the simultaneous (e.g., “one pot”) digestion of various regions of an mRNA, including but not limited to 5’UTR, ORF, and 3’UTR. Compositions described by the disclosure may contain between 2 and 100 isolated nucleic acids (e.g., between 2 and 100 guide strands). In some embodiments, a composition comprising a plurality of guide strands comprises 2, 3, 4, 5, 6, 7, 8, 9, or 10 unique isolated nucleic acid (e.g., guide strands). In some embodiments, a composition comprises three different isolated nucleic acids (e.g., guide strands). For example, using one, or two guide strands at a time (e.g. serially), multiple orthogonal digests of an mRNA can be performed in parallel with the same procedure and run time, allowing for greater sequence coverage during RNase mapping.

In some embodiments, the plurality comprises: (i) at least one isolated nucleic acid that results in cleavage of the mRNA 5’UTR, (ii) at least one isolated nucleic acid that results in cleavage of the mRNA 3’UTR; and, (iii) at least one isolated nucleic acid that results in cleavage of the mRNA ORF.

Once the signature of a mRNA sample is determined it can be compared with a known signature profile for a test mRNA. A “known signature profile for a test mRNA” as used herein refers to a control signature or fingerprint that uniquely identifies the test mRNA. The known signature profile for a test mRNA may be generated based on digestion of a pure sample and compared to the test signature profile. Alternatively it may be a known control signature, stored in a electronic or non-electronic data medium. For example, a control signature may be a theoretical signature based on predicted masses from the primary molecular sequence of a particular RNA (e.g., a test mRNA). In some embodiments, a control signature is produced by LC-MS/MS mRNA sequence mapping, for example as described in Example 7 below.

Various batches of mRNA (e.g., test mRNA) can be digested under the same conditions and compared to the signature of the pure mRNA to identify impurities or contaminants (e.g., additives, such as chemicals carried over from IVT reactions, or incorrectly transcribed mRNA) or to a known signature profile for the test mRNA. The identity of a test mRNA may be confirmed if the signature of the test mRNA shares identity with the known signature profile for a test mRNA. In some embodiments, the signature of the test mRNA shares at least 60%, at least 65%, at least 70%, at least 80%, at least 90%, at least 95%, at least 99%, or at least 99.9% identity with the known mRNA signature.

In some embodiments, various batches of mRNA can be digested under the same conditions in a high throughput fashion. For example, each mRNA sample of a batch may be placed in a separate well or wells of a multi-well plate and digested simultaneously with an RNase. A multi-well plate can comprise an array of 6, 24, 96, 384 or 1536 wells. However, the skilled artisan recognizes that multi-well plates may be constructed into a variety of other acceptable configurations, such as a multi-well plate having a number of wells that is a multiple of 6, 24, 96, 384 or 1536. For example, in some embodiments, the multi-well plate comprises an array of 3072 wells (which is a multiple of 1536). The number of mRNA samples digested simultaneously (e.g., in a multi-well plate) can vary. In some embodiments, at least two mRNA samples are digested simultaneously, In some embodiments, between 2 and 96 mRNA samples are digested simultaneously. In some embodiments, between 2 and 384 mRNA samples are digested simultaneously. In some embodiments, between 2 and 1536 mRNA samples are digested simultaneously. The skilled artisan recognizes that mRNA samples being digested simultaneously can each encode the same protein, or different proteins (e.g., mRNA encoding variants of the same protein, or encoding a completely different protein, such as a control mRNA).

As used herein, the term “digestion” refers to the enzymatic degradation of a biological macromolecule. Biological macromolecules can be proteins, polypeptides, or nucleic acids (e.g., DNA, RNA, mRNA), or any combination of the foregoing. Generally, the enzyme that mediates digestion is a protease or a nuclease, depending upon the substrate on which the enzyme performs its function. Proteases hydrolyze the peptide bonds that link amino acids in a peptide chain. Examples of proteases include but are not limited to serine proteases, threonine proteases, cysteine proteases, aspartase proteases, and metalloproteases. Nucleases cleave phosphodiester bonds between nucleotide subunits of nucleic acids. Generally, nucleases can be classified as deoxyribonucleases, or DNase enzymes (e.g., nucleases that cleave DNA), and ribonucleases, or RNase enzymes (e.g., nucleases that cleave RNA). Examples of DNase enzymes include exodeoxyribonucleases, which cleave the ends of DNA molecules, and restriction enzymes, which cleave specific sequences with a DNA sequence.

The amount of test mRNA that is digested can vary. In some embodiments that amount of test mRNA that is digested ranges from about 1 ng to about 100 µg. In some embodiments, the amount of test mRNA that is digested ranges from about 10 ng to about 80 µg. In some embodiments, the amount of test mRNA that is digested ranges from about 100 ng to about 1000 µg. In some embodiments, the amount of test mRNA that is digested ranges from about 500 ng to about 40 µg. In some embodiments, the amount of test mRNA that is digested ranges from about 1 µg to about 35 µg. In some embodiments, the amount of mRNA that is digested is about 1 µg, about 2 µg, about 3 µg, about 4 µg, about 5 µg, about 6 µg, about 7 µg, about 8 µg, about 9 µg, about 10 µg, about 11 µg, about 12 µg, about 13 µg, about 14 µg, about 15 µg, about 16 µg, about 17 µg, about 18 µg, about 19 µg, about 20 µg, about 21 µg, about 22 µg, about 23 µg, about 24 µg, about 25 µg, about 26 µg, about 27 µg, about 28 µg, about 29 µg, or about 30 µg.

The disclosure relates, in part, to the discovery that enzymes can be used to digest mRNA to create a unique population of RNA fragments, or a “signature”. Generally, any enzyme that digests (e.g., cleaves) bonds between ribonucleotides, for example a nuclease enzyme or a ribonuclease enzyme, may be used in methods described herein. Examples of nuclease enzymes include but are not limited to RNase enzymes, prokaryotic endonuclease enzymes (e.g., MazF, RecBCD endonuclease, T7 endonuclease, T4 endonuclease, Bal 31 endonuclease, micrococcal nuclease, etc.), tRNAse-type nuclease enzymes (e.g., colicin E5, colicin D, PrrC, etc.), and eukaryotic nuclease enzymes (e.g., Neospora endonuclease, S1-nuclease, P1-nuclease, mung bean nuclease 1, Ustilago nuclease, Endo R, etc.). In some embodiments, the enzyme is an RNase enzyme. Examples of RNase enzymes include but are not limited to RNase A, RNase H, RNase III, RNase L, RNase P, RNase E, RNase PhyM, RNase T1, RNase T2, RNase U2, RNase V, RNase PH, RNase R, RNase D, RNase T, polynucleotide phosphorylase (PNPase), oligoribonuclease, exoribonuclease I, exoribonuclease II, and cusativin.

In some embodiments, RNase T1 or RNase A is used to determine the identity of a test mRNA. In some embodiments, RNase H is used to determine the identity of a test mRNA. In some embodiments RNase T1 and cusativin are used to determine the identity of a test mRNA. In some embodiments, RNase T1 and cusativin are used in parallel to determine the identity of a test mRNA. Use of two or more enzymes “in parallel” may refer to the use of the enzymes in the same digest, or simultaneously in separate digests of the same test mRNA(s).

The concentration of RNase enzyme used in methods described by the disclosure can vary depending upon the amount of mRNA to be digested. However, in some embodiments, the amount of RNase enzyme ranges between about 0.1 Unit and about 500 Units of RNase. In some embodiments, the amount of RNase enzyme ranges from about 0.1 U to about 1 U, 1 U to about 5 U, 2 U to about 200 U, 10 U to about 450 U, about 20 U to about 400 U, about 30 U to about 350 U, about 40 U to about 300 U, about 50 U to about 250 U, or about 100 U to about 200 U.

The skilled artisan also recognizes that RNase enzymes can be derived from a variety of organisms, including but not limited to animals (e.g., mammals, humans, cats, dogs, cows, horses, etc.), bacteria (e.g., E. coli, S.aureus, Clostridiumspp., etc.), and mold (e.g., Aspergillusoryzae, Aspergillusniger, Dictyosteliumdiscoideum, etc.). RNase enzymes may also be recombinantly produced. For example, a gene encoding an RNase enzyme from one species (e.g., RNase T1 from A. oryzae) can be heterologously expressed in a bacterial host cell (e.g., E.coli) and purified. In some embodiments, the digestion is performed by an A. oryzae RNase T1 enzyme.

In some embodiments, the digestion is performed in a buffer. As used herein, the term “buffer” refers to a solution that can neutralize either an acid or a base in order to maintain a stable pH. Examples of buffers include but are not limited to Tris buffer (e.g., Tris-Cl buffer, Tris-acetate buffer, Tris-base buffer), urea buffer, bicarbonate buffer (e.g., sodium bicarbonate buffer), HEPES (4-2-hydroxyethyl-1-piperazineethanesulfonic acid) buffer, MOPS (3-(N-morpholino)propanesulfonic acid) buffer, PIPES (piperazine-N,N′-bis(2-ethanesulfonic acid)) buffer, and an ion pairing agent, such as Triethylammonium acetate (TEAAc buffer), DBAA, or other quaternary ammonium or phosphonium salts. A buffer can also contain more than one buffering agent, for example Tris-Cl and urea. The concentration of each buffering agent in a buffer can range from about 1 mM to about 10 M. In some embodiments, the concentration of each buffering agent in a buffer ranges from about 1 mM to about 20 mM, about 10 mM to about 50 mM, about 25 mM to about 100 mM, about 75 mM to about 200 mM, about 100 mM to about 500 mM, about 250 mM to about 1 M, about 500 mM to about 3 M, about 1 M to about 5 M, about 3 M to about 8 M, or about 5 M to about 10 M.

Generally, the pH maintained by a buffer can range from about pH 6.0 to about pH 10.0. In some embodiments, the pH can range from about pH 6.8 to about 7.5. In some embodiments, the pH is about pH 6.5, about pH 6.6, about pH 6.7, about pH 6.8, about pH 6.9, about pH 7.0, about pH 7.1, about pH 7.2, about pH 7.3, about pH 7.4, about pH 7.5, about pH 7.6, about pH 7.7, about pH 7.8, about pH 7.9, about pH 8.0, about pH 8.1, about pH 8.2, about pH 8.3, about pH 8.4, about pH 8.5, about pH 8.6, about pH 8.7, about pH 8.8, about pH 8.9, about pH 9.0, about pH 9.1, about pH 9.2, about pH 9.3, about pH 9.4, about pH 9.5, about pH 9.6, about pH 9.7, about pH 9.8, about pH 9.9, or about pH 10.

In some embodiments, a buffer further comprises a chelating agent. Examples of chelating agents include, but are not limited to, ethylenediaminetetraacetic acid (EDTA), ethylene glycol tetra acetic acid (EGTA), dimercapto succinic acid (DMSA), and 2,3-dimercapto-1-propanesulfonic acid (DMPS). In some embodiments, the chelating agent is EDTA (ethylenediaminetetraacetic acid). The concentration of EDTA can range from about 1 mM to about 500 mM. In some embodiments, the concentration of EDTA ranges from about 10 mM to about 300 mM. In some embodiments, the concentration of EDTA ranges from about 20 mM to about 250 mM EDTA.

The skilled artisan recognizes that to facilitate digestion, mRNA can be denatured prior to incubation with an RNase enzyme. In some embodiments, mRNA is denatured at a temperature that is at least 50° C., at least 60° C., at least 70° C., at least 80° C., or at least 90° C. Digestion of a test mRNA can be carried out at any temperature at which the RNase enzyme will perform its intended function. The temperature of a test mRNA digestion reaction can range from about 20° C. to about 100° C. In some embodiments, the temperature of a test mRNA digestion reaction ranges from about 30° C. to about 50° C. In some embodiments, a test mRNA is digested by an RNase enzyme at 37° C.

Digestion with RNase enzymes may lead to the formation of cyclic phosphates and other intermediates (e.g., 2′ or 3′-phosphates) that can interfere with downstream processing (e.g., detection of digested test mRNA fragments). Thus, in some embodiments, an mRNA digestion buffer further comprises agents that disrupt or prevent the formation of intermediates. In some embodiments, the buffer further comprises 2′,3′-Cyclic-nucleotide 3′-phosphodiesterase (CNP) and/or Alkaline Phosphatase, such as Calf Intestinal Alkaline Phosphatase (CIP), or Shrimp Alkaline Phosphatase (SAP). The concentration of each agent that disrupts or prevents formation of intermediates can range from about 10 ng/µL to about 100 ng/µL. In some embodiments, the concentration of each agent ranges from about 15 ng/µL to about 25 ng/ µL. Alternatively, or in combination with the above-stated concentration range, the amount of agent can range from about 1 U to about 50 U, about 2 U to about 40 U, about 3 U to about 35 U, about 4 U to about 30 U, about 5 U to about 25 U, or about 10 U to about 20 U. In some embodiments, digestion with RNase enzymes is performed in a digestion buffer not containing CIP and/or CNP.

In some embodiments, a buffer further comprises magnesium chloride (MgCl₂) Generally, MgCl₂ can act as a cofactor for enzyme (e.g., RNase) activity. The concentration of MgCl₂ in the buffer ranges from about 0.5 mM to about 200 mM. In some embodiments, the concentration of MgCl₂ in the buffer ranges from about 0.5 mM to about 10 mM, 1 mM to about 20 mM, 5 mM to about 20 mM, 10 mM to about 75 mM, or about 50 mM to about 150 mM. In some embodiments, the concentration of MgCl₂ in the buffer is about 1 mM, about 5 mM, about 10 mM, about 50 mM, about 75 mM, about 100 mM, about 125 mM, or about 150 mM.

In some embodiments, digestion of a test mRNA comprises two incubation steps: (a) RNase digestion of test mRNA, and (b) processing of digested test mRNA. In some embodiments, digestion of a test mRNA further comprises the step of denaturing test mRNA prior to digestion. The incubation time for each of the above steps (a), (b), and (c) can range from about 1 minute to about 24 hours. In some embodiments, incubation time ranges from about 1 minute to about 10 minutes. In some embodiments, incubation time ranges from about 5 minutes to about 15 minutes. In some embodiments, incubation time ranges from about 30 minutes to about 4 hours (240 minutes). In some embodiments, incubation time ranges from about 1 hour to about 5 hours. In some embodiments, incubation time ranges from about 2 hours to about 12 hours. In some embodiments, incubation time ranges from about 6 hours to about 24 hours.

The skilled artisan recognizes that digestions may be carried out under various environmental conditions based upon the components present in the digestion reaction. Any suitable combination of the foregoing components and parameters may be used. For example, digestion of a test mRNA may be carried out according to the protocol set forth in Table 1.

In some aspects, the disclosure provides a “one-pot” RNase H digestion assay for characterization of nucleic acids (e.g., a test mRNA). Generally, RNase H digestion assays comprise separate steps for (i) annealing a guide strand to a target mRNA and (ii) digesting the guide strand-mRNA duplex. The disclosure relates, in part, to the discovery that guide strand annealing and RNase H digestion steps can be combined into a single step when appropriate conditions (e.g., as set forth in Table 1) are provided. Without wishing to be bound by any particular theory, a one-pot RNase H digestion assay as described by the disclosure, in some embodiments, has a reduced run time and provides higher quality samples for analytical methods (e.g., HPLC/MS, etc.) than methods requiring multiple steps (e.g., separate annealing and digestion steps, etc.).

A “fragment” of a polynucleotide of interest comprises a series of consecutive nucleotides from the sequence of said test RNA. By way of example, a “fragment” of a polynucleotide of interest may comprise (or consist of) at least 1 at least 2, at least 5, at least 10, at least 20, at least 30 consecutive nucleotides from the sequence of the polynucleotide (e.g., at least 1 at least 2, at least 5, at least 10, at least 20, at least 30, at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment of a polynucleotide (e.g., an mRNA fragment) can consist of the same nucleotide sequence as another fragment, or consist of a unique nucleotide sequence.

A “plurality of mRNA fragments” refers to a population of at least two mRNA fragments. mRNA fragments comprising the plurality can be identical, unique, or a combination of identical and unique (e.g., some fragments are the same and some are unique). The skilled artisan recognizes that fragments can also have the same length but comprise different nucleotide sequences (e.g., CACGU, and AAAGC are both five nucleotides in length but comprise different sequences). In some embodiments, a plurality of mRNA fragments is generated from the digestion of a single species of mRNA. A plurality of mRNA fragments can be at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, or at least 500 mRNA fragments. In some embodiments, a plurality of mRNA fragments comprises more than 500 mRNA fragments.

The plurality of fragments is physically separated. As used herein, the term “physically separated” refers to the isolation of mRNA fragments based upon a selection criteria. For example, a plurality of mRNA fragments resulting from the digestion of a test mRNA can be physically separated by chromatography or mass spectrometry. In some embodiments, fragments of a test mRNA can be physically separated by capillary electrophoresis to generate an electropherogram. Examples of chromatography methods include size exclusion chromatography and high performance liquid chromatography (HPLC). Examples of mass spectrometry physical separation techniques include electrospray ionization mass spectrometry (ESI-MS) and matrix-assisted laser desorption ionization mass spectrometry (MALDI-MS). In some embodiments, each of fragment of the plurality of mRNA fragments is detected during the physical separation. For example, a UV spectrophotometer coupled to an HPLC machine can be used to detect the mRNA fragments during physical separation (e.g., a UV absorbance chromatogram). A mass spectrometer coupled to an HPLC can also be used to subject chromatographically-separated mRNA fragments to a second dimension of separation, as well as detection. The resulting data, also called a “trace” provides a graphical representation of the composition of the plurality of mRNA fragments. In another embodiment, a mass spectrometer generates mass data during the physical separation of a plurality of mRNA fragments. The graphic depiction of the mass data can provide a “mass fingerprint” that identifies the contents of the plurality of mRNA fragments.

Mass spectrometry encompasses a broad range of techniques for identifying and characterizing compounds in mixtures. Different types of mass spectrometry-based approaches may be used to analyze a sample to determine its composition. Mass spectrometry analysis involves converting a sample being analyzed into multiple ions by an ionization process. Each of the resulting ions, when placed in a force field, moves in the field along a trajectory such that its acceleration is inversely proportional to its mass-to-charge ratio. A mass spectrum of a molecule is thus produced that displays a plot of relative abundances of precursor ions versus their mass-to-charge ratios. When a subsequent stage of mass spectrometry, such as tandem mass spectrometry, is used to further analyze the sample by subjecting precursor ions to higher energy, each precursor ion may undergo disassociation into fragments referred to as product ions. Resulting fragments can be used to provide information concerning the nature and the structure of their precursor molecule.

MALDI-TOF (matrix-assisted laser desorption ionization time of flight) mass spectrometry provides for the spectrometric determination of the mass of poorly ionizing or easily-fragmented analytes of low volatility by embedding them in a matrix of light-absorbing material and measuring the weight of the molecule as it is ionized and caused to fly by volatilization. Combinations of electric and magnetic fields are applied on the sample to cause the ionized material to move depending on the individual mass and charge of the molecule. U.S. Pat. No. 6,043,031, issued to Koster et al., describes an exemplary method for identifying single-base mutations within DNA using MALDI-TOF and other methods of mass spectrometry.

HPLC (high performance liquid chromatography) is used for the analytical separation of bio-polymers, based on properties of the bio-polymers. HPLC can be used to separate nucleic acid sequences based on size and/or charge. A nucleic acid sequence having one base pair difference from another nucleic acid can be separated using HPLC. Thus, nucleic acid samples, which are identical except for a single nucleotide may be differentially separated using HPLC, to identify the presence or absence of a particular nucleic acid fragments. Preferably the HPLC is HPLC-UV.

The data generated using the methods of the invention can be processed individually or by a computer. For instance, a computer-implemented method for generating a data structure, tangibly embodied in a computer-readable medium, representing a data set representative of a signature profile of an RNA sample may be performed according to the invention.

Some embodiments relate to at least one non-transitory computer-readable storage medium storing computer-executable instructions that, when executed by at least one processor, perform a method of identifying an RNA in a sample.

Thus, some embodiments provide techniques for processing MS/MS data that may identify impurities in a sample with improved accuracy, sensitivity and speed. The techniques may involve structural identification of an RNA fragment regardless of whether it has been previously identified and included in a reference database. A scoring approach may be utilized that allows determining a likelihood of an impurity being present in a sample, with scores being computed so that they do not depend on techniques used to acquire the analyzed mass spectrometry data.

In some embodiments the known signature profile for known mRNA data may be computationally generated, or computed, and stored, for example, in a first database. The first database may store any type of information on the RNA, including an identifier of each RNA fragment to form a complete signature and any other suitable information. In some embodiments, a score may be computed for each set of computed fragments retrieved from a second database including the known signatures, the score indicating correlation between the set of known signatures and the set of experimentally obtained fragments. To compute the score, for example, each fragment in a set of computed fragments matching a corresponding fragment in the set of experimentally obtained fragments may be assigned a weight based on a relative abundance of the experimentally obtained fragment. A score may thus be computed for each set of computed fragments based on weights assigned to fragments in that set. The scores may then be used to identify difference between the RNA sample and the known sequence.

A computer system that may implement the above as a computer program typically may include a main unit connected to both an output device which displays information to a user and an input device which receives input from a user. The main unit generally includes a processor connected to a memory system via an interconnection mechanism. The input device and output device also may be connected to the processor and memory system via the interconnection mechanism.

An illustrative implementation of a computer system that may be used in connection with some embodiments may be used to implement any of the functionality described above. The computer system may include one or more processors and one or more computer-readable storage media (i.e., tangible, non-transitory computer-readable media), e.g., volatile storage and one or more non-volatile storage media, which may be formed of any suitable data storage media. The processor may control writing data to and reading data from the volatile storage and the non-volatile storage device in any suitable manner, as embodiments are not limited in this respect. To perform any of the functionality described herein, the processor may execute one or more instructions stored in one or more computer-readable storage media (e.g., volatile storage and/or non-volatile storage), which may serve as tangible, non-transitory computer-readable media storing instructions for execution by the processor.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation comprises at least one computer-readable storage medium (i.e., at least one tangible, non-transitory computer-readable medium), such as a computer memory (e.g., hard drive, flash memory, processor working memory, etc.), a floppy disk, an optical disk, a magnetic tape, or other tangible, non-transitory computer-readable medium, encoded with a computer program (i.e., a plurality of instructions), which, when executed on one or more processors, performs above-discussed functions. The computer-readable storage medium can be transportable such that the program stored thereon can be loaded onto any computer resource to implement techniques discussed herein. In addition, it should be appreciated that the reference to a computer program which, when executed, performs above-discussed functions, is not limited to an application program running on a host computer. Rather, the term “computer program” is used herein in a generic sense to reference any type of computer code (e.g., software or microcode) that can be employed to program one or more processors to implement above-techniques.

### EXAMPLES

### Example 1: RNase Mapping/Fingerprinting Example Protocol

Table 1 (below) demonstrates an example protocol for RNase digestion:

TABLE 1

Example protocol for RNase T1 digestion.

| RNase T1 Fingerprint with UREA Buffer | | Concentration | Source |
|---|---|---|---|
| 10.0 µl | mRNA | 3 mg/ml | |
| 15.0 µl | UREA Solution, Sigma | 8000 mM | UREA Solution 8 M, Sigma 51457 |
| 3.0 µl | Tris, pH 7 | 1000 mM | Tris-Cl Buffer, pH 7, Sigma, T1819 |
| 2.0 µl | EDTA | 50 mM | EDTA, 0,5 M, pH 8, Applichem, A4892.0500 |
| → 10 min @ 90° C. | | | |
| 20.0 µ1 | RNase T1 | 10.0 U/µl | RNase, T1, Thermo, #EN0542 |
| →3 hr @ 37° C. | | | |
| 2.0 µl | CNP | 0.040 µg/µl | CNP, Origene, TP602895 |
| 2.0 µl | MgCI₂ | 100 mM | MgCI2, 1 M, Ambion, AM9530G |
| →1 h @37° C. | | | |
| 2.0 µl | CIP | 10.0 U/µl | CIP, New England BioLabs, M0290L |
| →1 h @ 37° C. | | | |
| Stop Incubation | | | |
| 5.0 µl | 250 mM EDTA, 1M TEAAc | | |
| 61.0 µl | Total Sample Volume | | |

Briefly, a mRNA sample was denatured at high temperature in a urea buffer. RNase (e.g., RNase T1) was added to the denatured sample and incubated. 2′,3′-phosphates were digested for 1 hour with cyclic-nucleotide 3′-phosphodiesterase (CNP) at 37° C. The resultant 2′- or 3′ phosphates were removed by digestion with Calf Intestinal Alkaline Phosphatase (CIP). The digestion was stopped by the addition of EDTA. TEAAc was also added for strong adsorption on the HPLC column. After the reaction was stopped, the digested mRNA sample was prepared for analysis using HPLC. Suitable analysis methods include IP-RP-HPLC, HPLC-UV, AEX-HPLC, HPLC-ESI-MS and/or MALDI-MS, some of which are described below.

### Identification of RNA Using RNase Fingerprinting

A first mRNA sample (sample 1) was processed according the methods described above. A table summarizing theoretical RNase T1 cleavage products from that analysis is provided below in Table 2.

TABLE 2

Theoretical RNase T1 cleavage products.

| | # Unique Fragments | Prevalence |
|---|---|---|
| 1 mers | 1 | 152 |
| 2 mers | 4 | 92 |
| 3 mers | 9 | 71 |
| 4 mers | 20 | 52 |
| 5 mers | 23 | 29 |
| 6 mers | 31 | 34 |
| 7 mers | 23 | 24 |
| 8 mers | 18 | 18 |
| 9 mers | 10 | 10 |
| 10 mers | 7 | 7 |
| 11 mers | 8 | 8 |
| 12 mers | 3 | 3 |
| 13 mers | 3 | 3 |
| 14 mers | 1 | 1 |
| 15 mers | 1 | 1 |
| 16 mers | 2 | 2 |
| 17 mers | - | - |
| 18 mers | 1 | 1 |
| 19 mers | - | - |
| 20 mers | - | - |
| 21 mers | - | - |
| 22 mers | - | - |
| 23 mers | - | - |
| 24 mers | 1 | 1 |
| 25 mers | 1 | 1 |
| 26 mers | 1 | 1 |
| 27 mers | - | - |
| 28 mers | - | - |
| 29 mers | 1 | 1 |
| 106 mers | 1 | 1 |

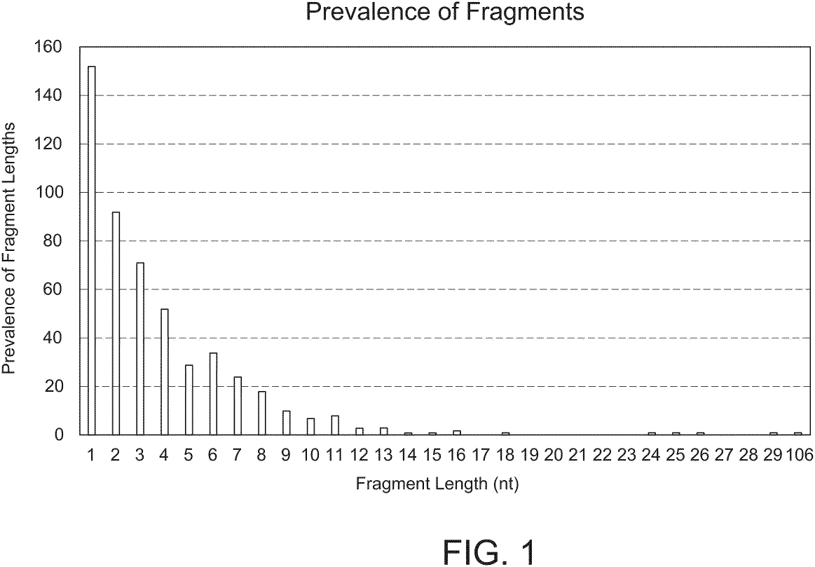

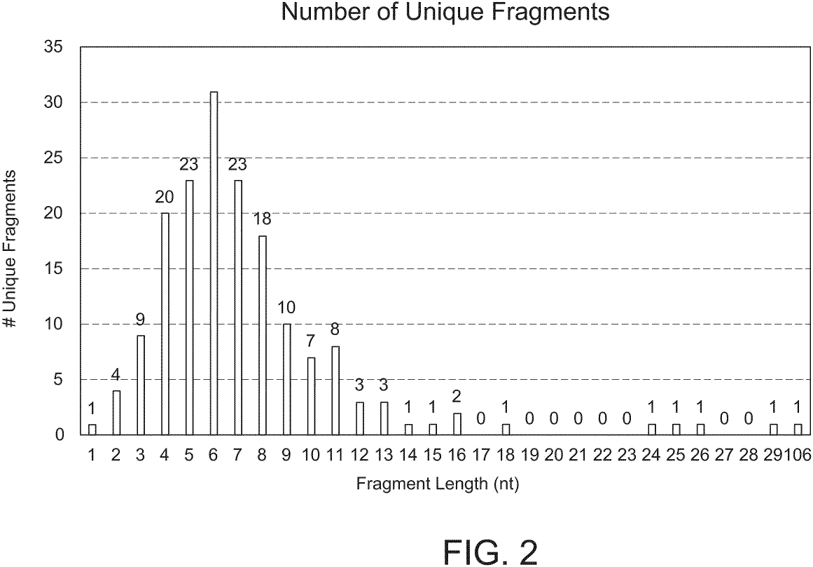

The prevalence of those predicted fragments and the number of unique fragments identified in the mRNA are show in FIGS. 1-2. For example, there are 92 2-mer fragments generated by this digestion as shown in FIG. 1. There are 31 unique 6-mer fragments generated by this RNase digestion, as shown in FIG. 2.

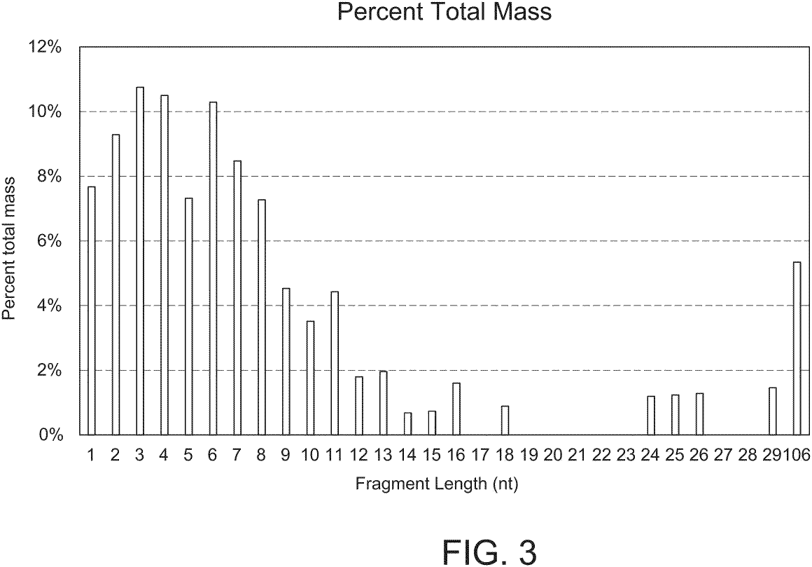

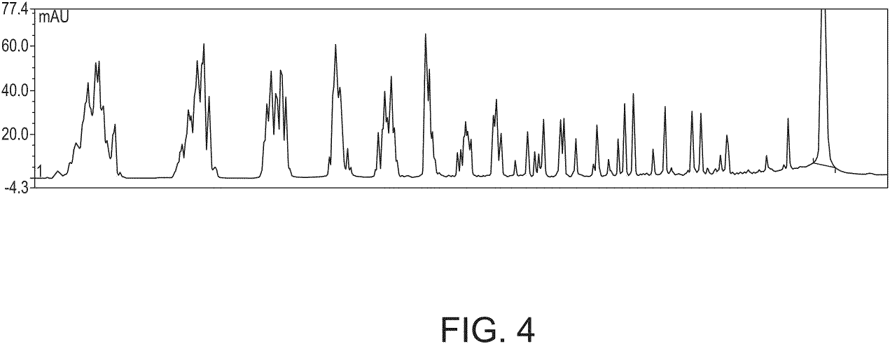

The percent total mass of different fragment lengths is shown in the graph of FIG. 3. For example, 10% of the total mass of the test mRNA sample is digested into 6-mers. FIG. 4 shows analyses of Sample 1 after RNase T1 digestion by HPLC produces a chromatographic pattern that represents a unique fingerprint for Sample 1.

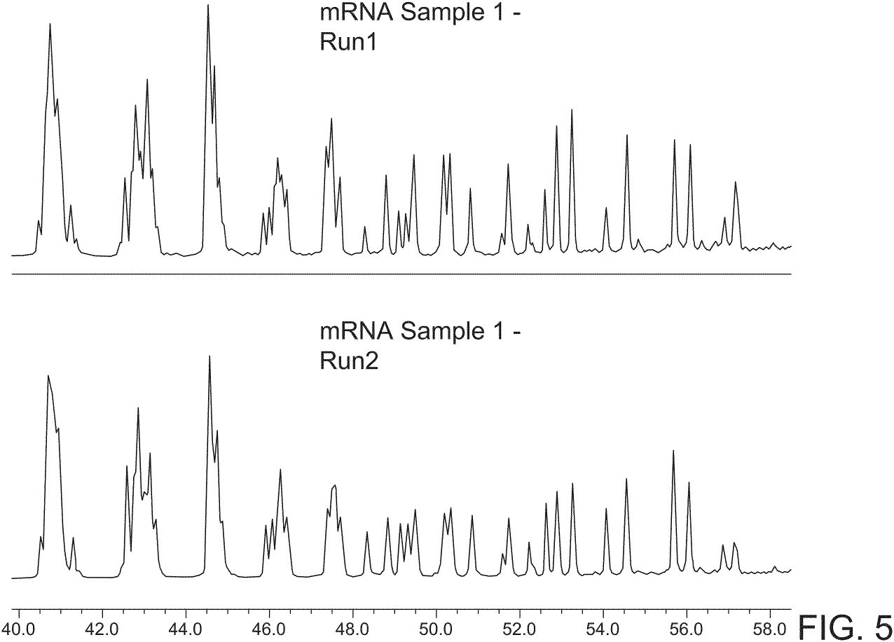

Two test samples of mRNA Sample 1 were digested and run on an HPLC column. FIG. 5 shows representative HPLC data demonstrating the reproducibility of the RNase digestion. The trace patterns for each digestion of mRNA Sample 1 (e.g., Run 1 and Run 2) are almost identical

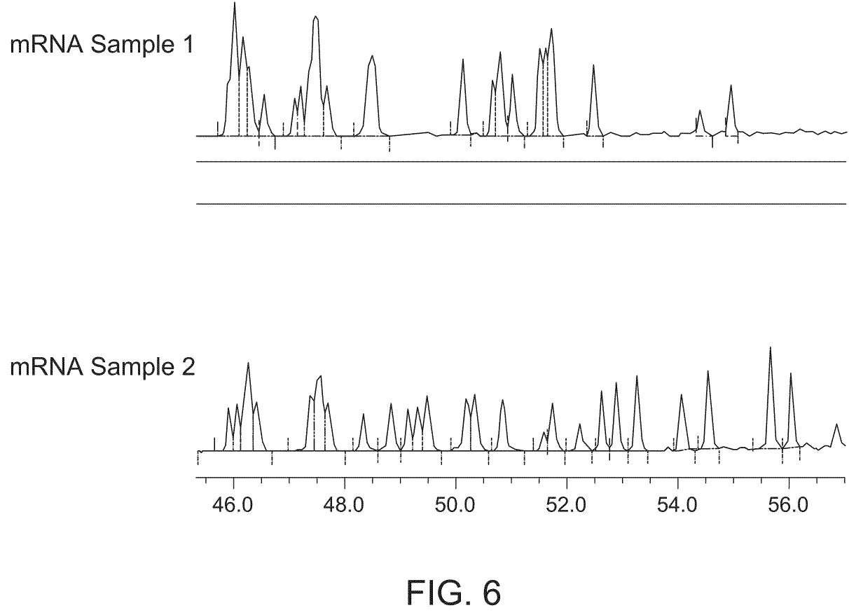

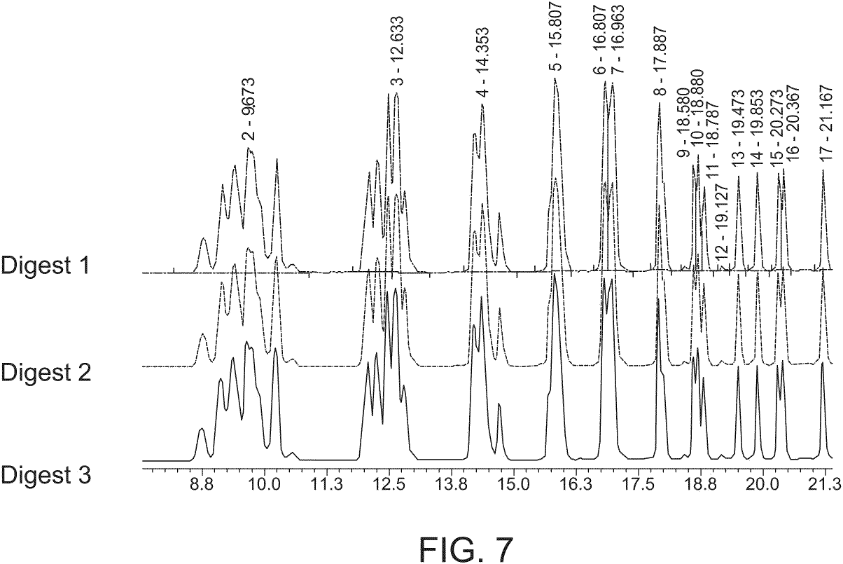

The methods were also performed on different mRNA samples. FIG. 6 shows representative HPLC data demonstrating the unique pattern generated by RNase digestion of two different mRNA samples (e.g., mRNA Sample 1 and mRNA Sample 2). FIG. 7 shows representative HPLC data demonstrating the reproducibility of RNase digestion across multiple digests. Separate aliquots of mRNA Sample 3 were RNase digested (Digest 1, 2 and 3) and run on an HPLC column. The trace patterns for each digestion are almost identical

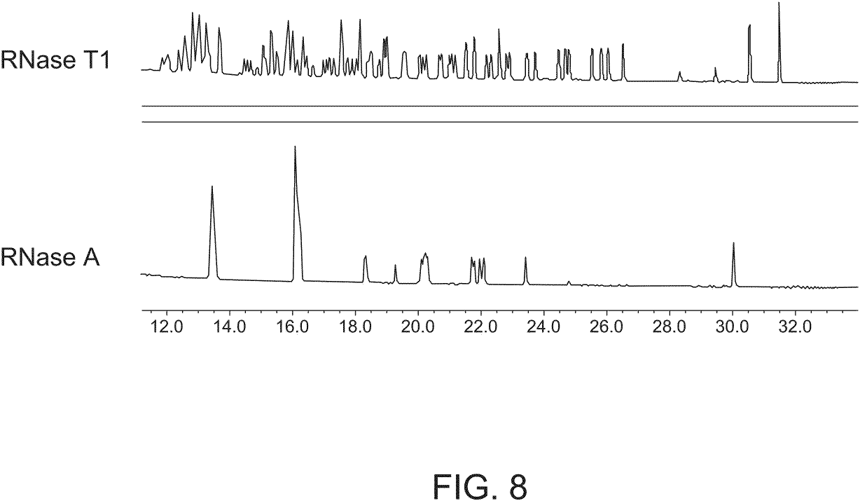

The effect of different RNase enzymes on the analysis methods was also examined. The methods were performed using RNase T1 and RNase A. FIG. 8 shows representative HPLC data illustrating that digestion with different RNase enzymes (e.g., RNase T1 or RNase A) leads to the generation of distinct trace patterns. Digestion of mRNA Sample 3 with RNase T1 provided a more detailed trace pattern than digestion with RNase A.

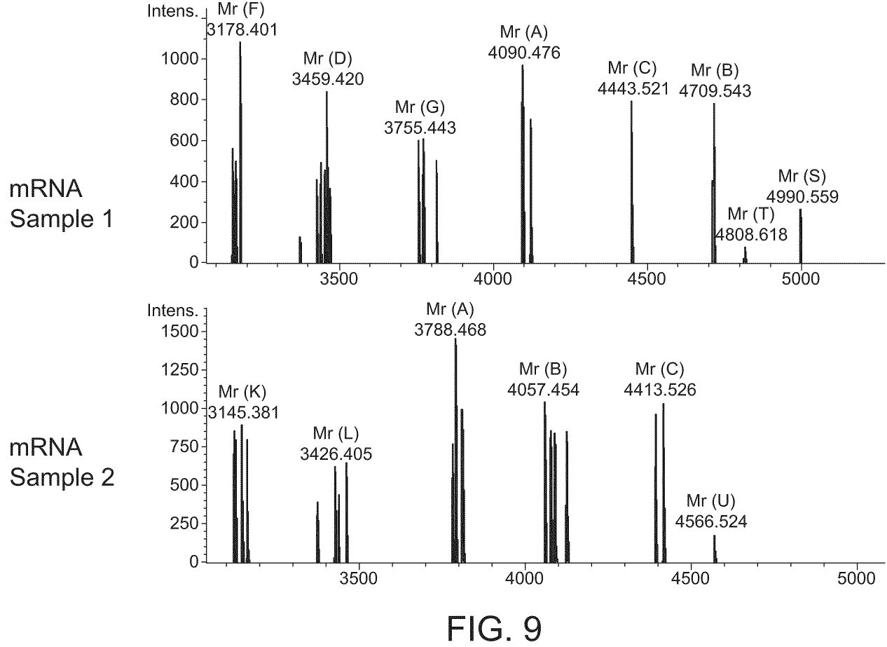

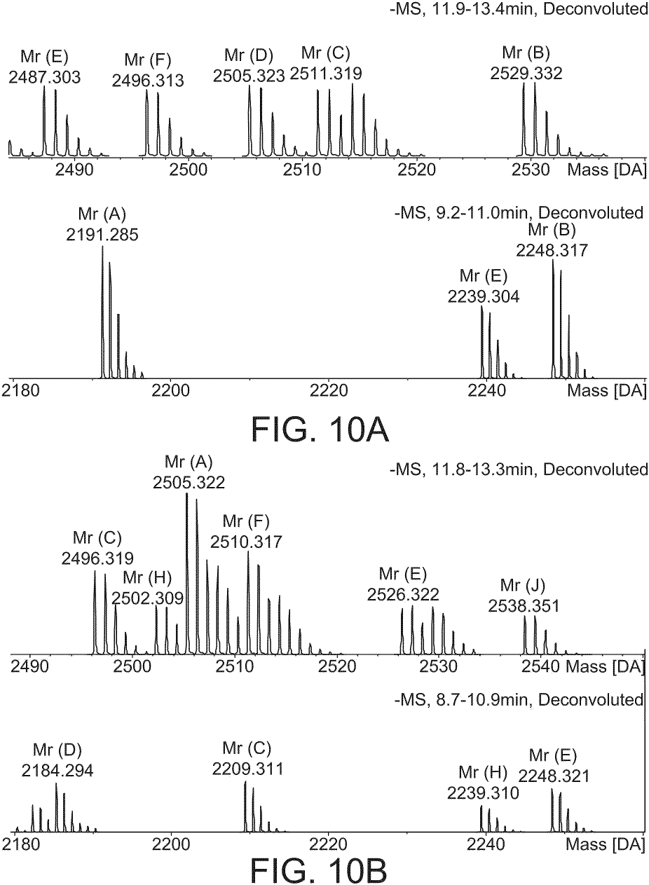

The methods were also performed using different analysis techniques. FIG. 9 shows representative ESI-MS data. Two mRNA samples (mRNA Sample 1 and mRNA Sample 2) were digested with RNase T1. ESI-MS was performed on digested samples. Results demonstrated that unique mass traces are generated for each sample. FIGS. 10A-10B show representative data from ESI-MS of two RNase T1-digested mRNA samples (mRNA Sample 4 and mRNA Sample 5). Data demonstrated that each mass fingerprint is unique.

### Example 2: RNase Mapping/Fingerprinting of mCherry mRNA

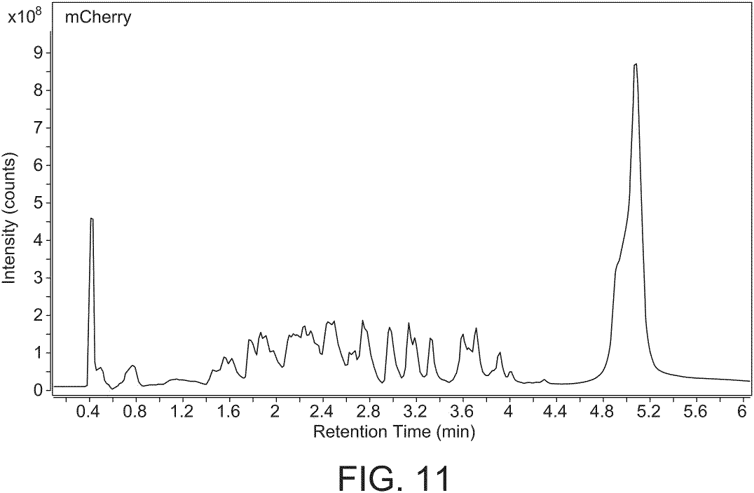

A mRNA sample encoding the fluorescent protein mCherry was processed according the methods described above and LC/MS was performed. Representative data of the LC/MS is shown in FIG. 11.

A total of 43 different oligonucleotide masses were detected. Of these 43 oligos, 28 were unique to a specific location on the mCherry sequence, while 15 were positively identified but could not be localized to a specific location (due to the presence of the same oligo, or isomers thereof, at different locations within the mCherry sequence). Representative data related to the prevalence of digested oligonucleotide fragments and the number of unique fragments identified in the mRNA are show in Table 3. For example, there are 38 2-mer fragments generated by this digestion. There are 5 unique 9-mer fragments generated by this RNase digestion.

TABLE 3

Oligonucleotide fragments produced by RNase T1 digestion of mCherry mRNA.

| | # Unique Fragments | | Prevalence |
|---|---|---|---|
| 2 mers | 0 | 38 | |
| 3 mers | 0 | 23 | |
| 4 mers | 2 | 2 | |
| 5 mers | 4 | 4 | |
| 6 mers | 1 | 1 | |
| 7 mers | 5 | 5 | |
| 8 mers | 5 | 5 | |
| 9 mers | 5 | 5 | |
| 10 mers | 3 | 3 | |
| 12 mers | 2 | 2 | |
| 13 mers | 1 | 1 | |
| 14 mers | 4 | 4 | |
| 16 mers | 2 | 2 | |
| 18 mers | 1 | 1 | |
| 22 mers | 2 | 2 | |
| 24 mers | 1 | 1 | |
| 140 mers | 1 | 1 | |

Table 4 shows representative data relating to the mass (Da) of the unique fragments identified by RNase T1 digestion of mCherry mRNA.

TABLE 4

Mass of representative mCherry oligonucleotides

| MASS (Da) | RET. TIME (mins) | SEQUENCES |
|---|---|---|
| Unique Sequences | | |
| 1599.3 | 1.61 | AAAAG |
| | | UAAG |
| 2897.49 | 2.78 | AAAUAUAAG |
| | | AUCAUCAAG |
| 1579.31 | 1.55 | ACACG |
| 2209.39 | 2.31 | CCCUAUG |
| | | ACCACUUCCUUUCG (SEQ ID NO: 1) |
| 1241.24 | 1.28 | CCUG |
| | | AUAUUCCUG |
| 2539.43 | 2.43 | ACUAUCUG |
| | | CUUUCCCG |
| 2220.38 | 2.31 | AACUUUG |
| | | UAACCCAAG |
| 2549.43 | 2.46 | ACAUUAUG |
| | | ACAUACAAAG (SEQ ID NO: 2) |
| 1928.35 | 2 | AAAAAG |
| | | UAUAAUG |
| 2887.49 | 2.85 | AAUAUCAAG |
| | | AUAUUACUUCACACAAUG (SEQ ID NO: 3) |
| 1589.3 | 1.58 | AACAG |
| | | UACAAAUG |
| 2239.38 | 2.23 | AUAAUAG |
| 1560.3 | 1.5 | CCUCG |
| | | CUUCUUG |
| 3829.67 | 3.03 | GCCUCCCCCCAG (SEQ ID NO: 4) |
| | | CCCCUCCUCCCCUUCCUGCACCCG (SEQ ID NO: 5) |
| 2527.47 | 2.31 | UACCCCCG |
| 46346.1 | 5.09 | C(A₁₄₀) (SEQ ID NO: 6) |

The combined length of all unique oligos was 373 nt, out of a total mRNA length of 1014 nt. Thus, the sequence coverage of the mCherry mRNA by unique oligos was 373/1014 = 36.8%. Oligos identified by RNase T1 digest of mCherry are shown in Table 5. When non-unique oligos were considered as well, the sequence coverage jumped to anywhere from 43.9% to 63.8%, depending on whether each identified non-unique oligo originated from just one possible location, or all of the possible locations combined.

### Example 3: mRNA Characterization by RNase Fingerprinting/Mapping

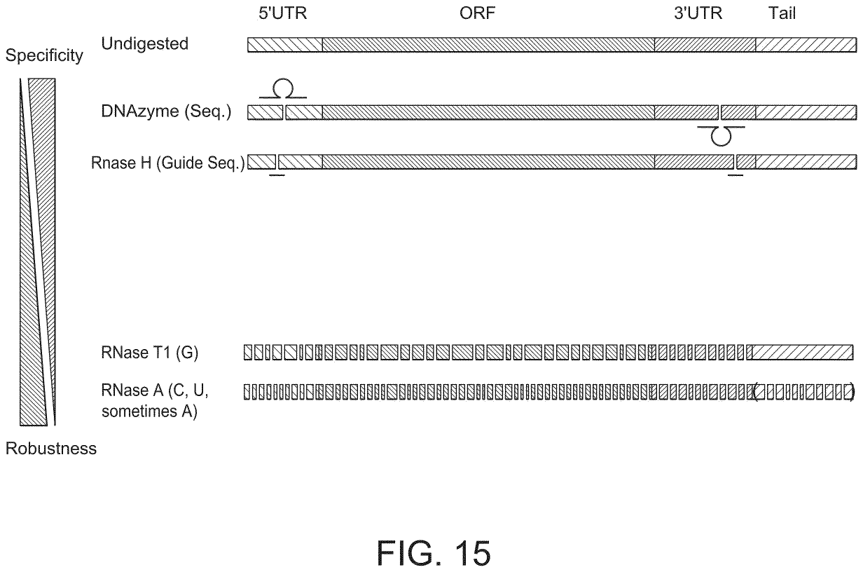

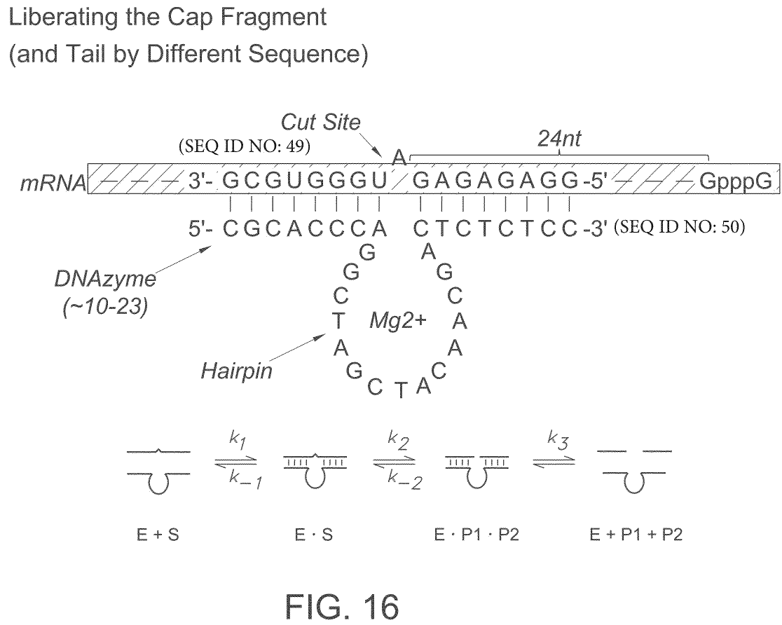

In some embodiments, assays for mRNA characterization described by this disclosure include a digestion step during sample preparation. Generally, these digestions cover a spectrum from specific and qualitative to non-specific and quantitative (FIG. 15); in that order they are digestion by DNAzyme, RNase H, RNase T1 and RNase A. This example describes the digestion of mRNA Cap, open reading frame (ORF) and poly A tail (also referred to as “Tail”) for mRNA fingerprinting/mapping.

### 1. mRNA Cap and Tail Digestion

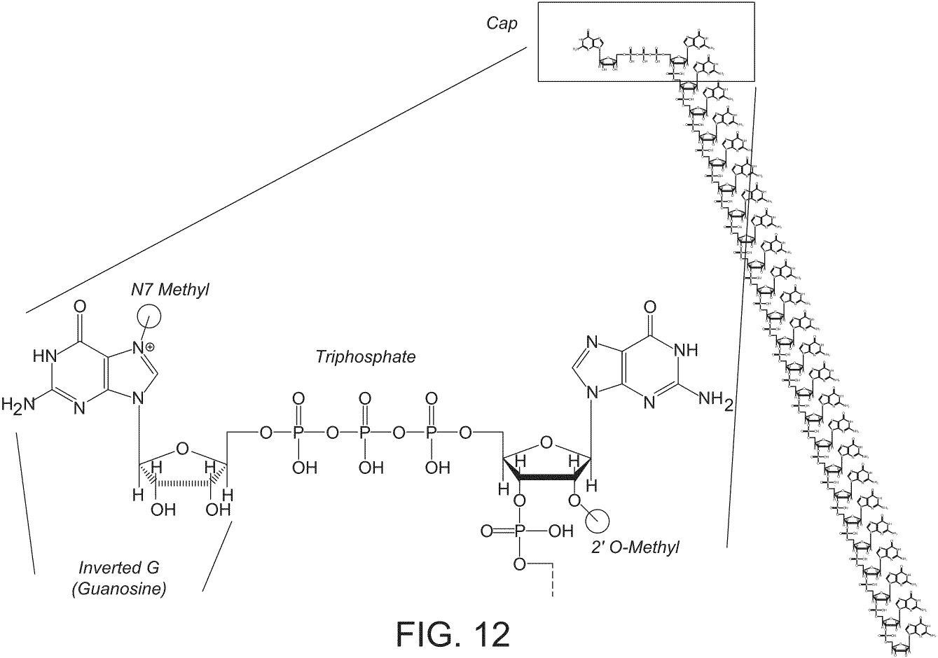

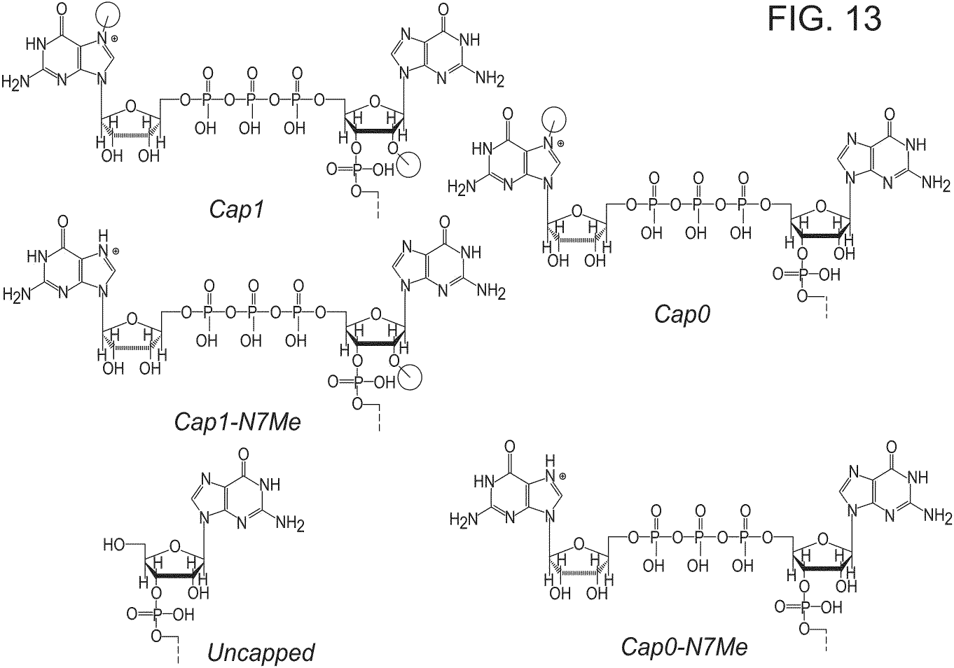

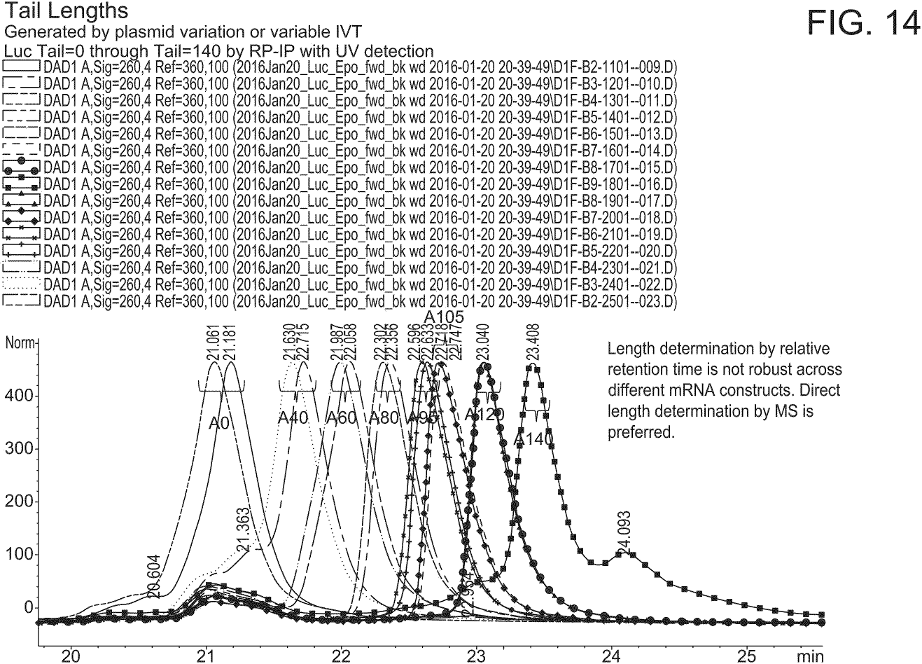

mRNA capping is a process by which the 5′end of the mRNA is modified with a 7-methylguanylate cap (also referred to as “Cap”) to create stable and mature messenger RNA able to undergo translation during protein synthesis. A schematic illustration of Cap is shown in FIG. 12. In certain cases, the mRNA capping process is incomplete, leaving mRNA having a partial Cap (e.g., Cap that is not methylated at position 7) or uncapped mRNA. Examples of partial Cap and uncapped structures are shown in FIG. 13. In some embodiments, it is desirable to map the 5′ UTR of an mRNA to identify whether the mRNA contains Cap, partial Cap, or is uncapped. Similarly, in some embodiments, it is desirable to characterize the 3′ UTR of an mRNA, for example to quantify the length of the mRNA polyA tail (also referred to as “Tail”).

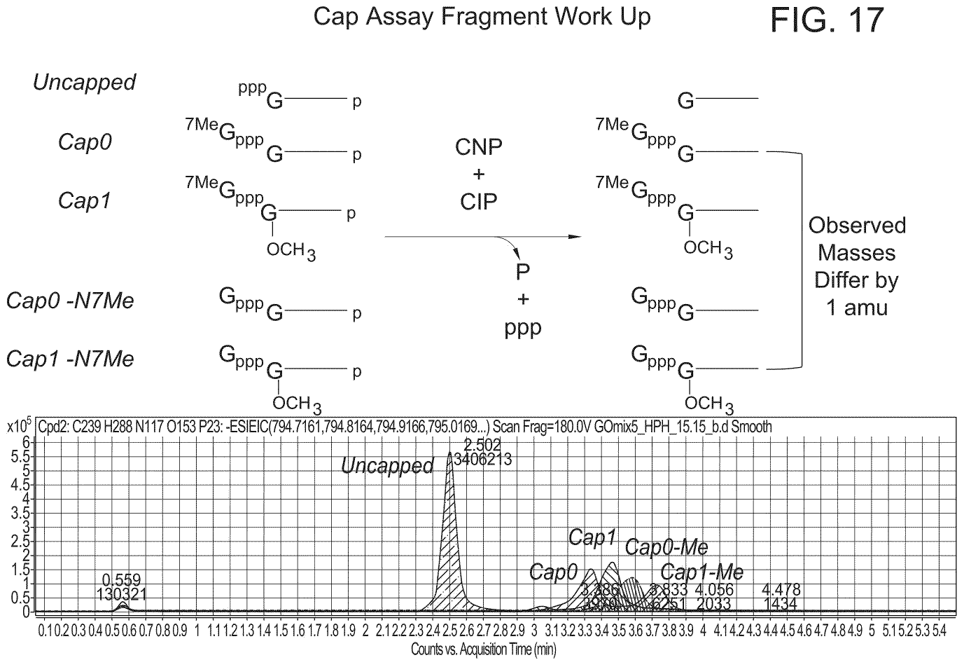

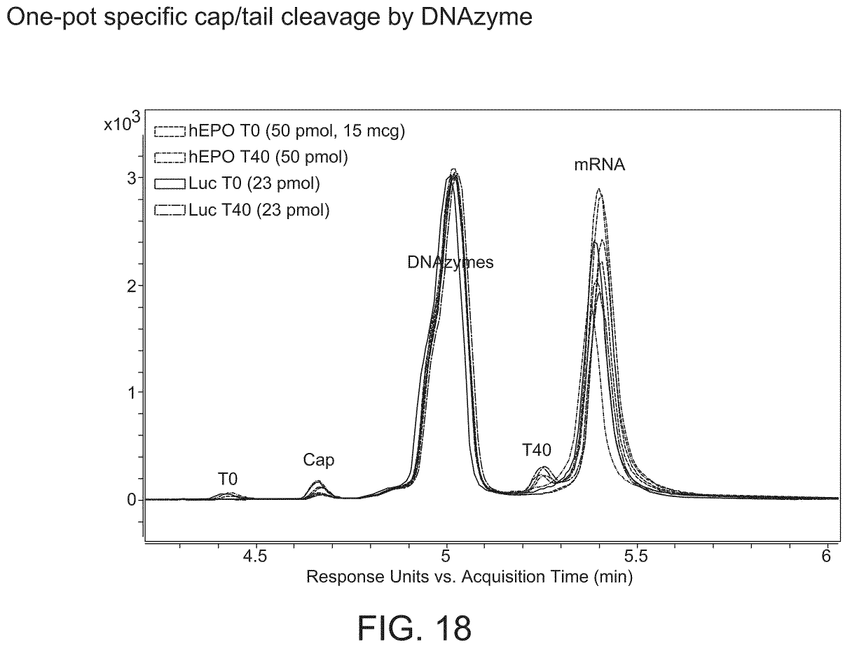

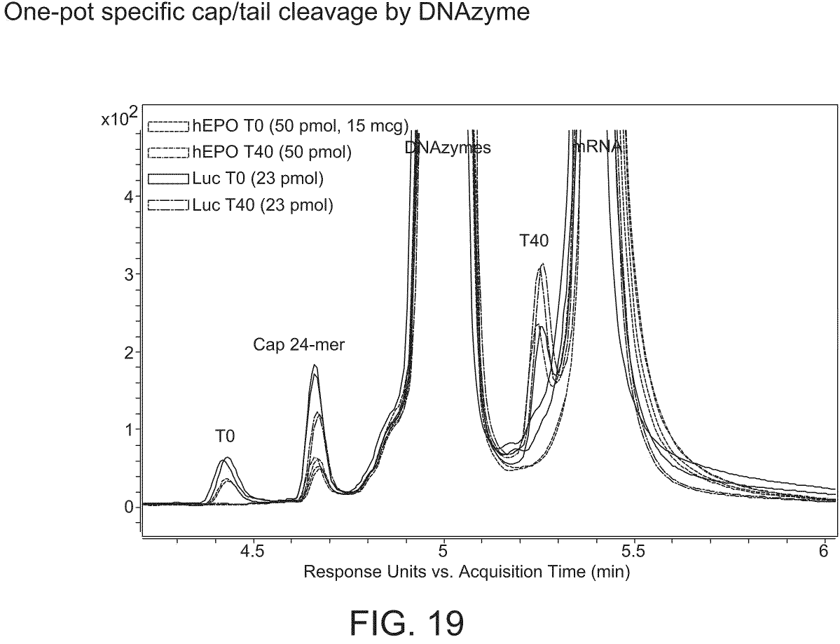

DNAzyme performs sequence specific cleavage of the 3′ and/or 5′ UTR of mRNA to allow measurement of Cap and Tail by mass spec (FIG. 16 and FIG. 17). However, redesigning the DNAzyme is a slow process and does not allow for UTR variation. DNAzyme digestions are not total and sometimes fail due to sequence and/or secondary structure. For example, FIGS. 18 and 19 show representative data of a one-pot specific Cap/tail cleavage of mRNA using DNAzyme. Data indicate that undigested mRNA and tail species co-elute due to the hydrophobicity of the polyA tail, which may bias quantitation of certain tail lengths.

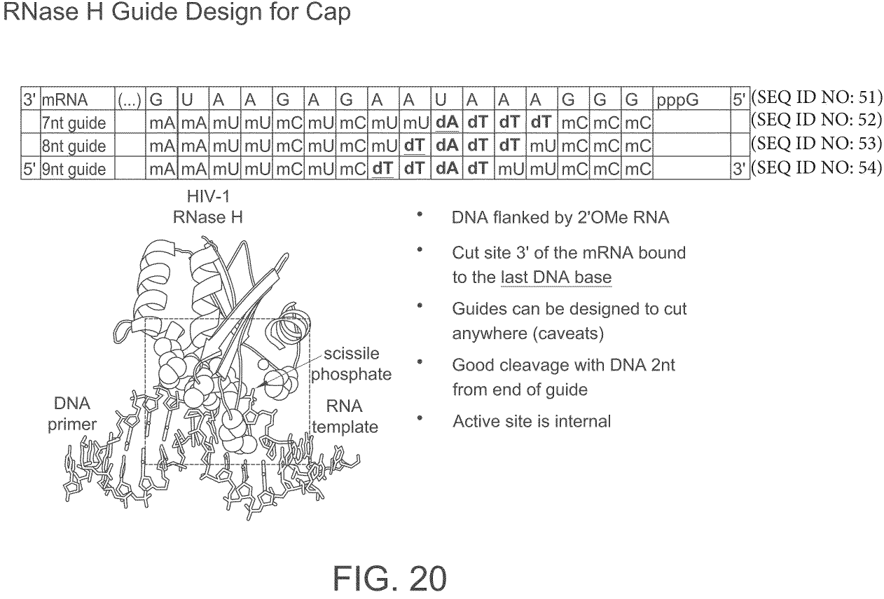

RNase H also performs sequence specific cleavage of the 3′ and/or 5′ UTR of mRNA by recognizing a complementary guide strand bound to the mRNA (FIG. 20). The guide strand is composed of four DNA nucleotides (e.g., 2′-deoxyribonucleotides, such as “dT”, “dG”, “dC”, dA”) flanked by 2′O-methyl RNA (e.g., “mU”, “mG”, “mC”, mA”). Cleavage occurs on the mRNA to the 5′ of the four DNA bases (e.g., to the 3′ of the mRNA base paired with the final DNA base). FIG. 20 provides three non-limiting examples of RNase H guide strands designed to target a mRNA Cap sequence. Further non-limiting examples of RNase H guide strands are provided in Table 5, shown below. A non-limiting example of an RNase H digestion protocol is shown in Table 6.

TABLE 5

Non-limiting examples of Cap-targeting RNase H guide strands

| | |
|---|---|
| Cap Guide | Name |
| mCmAmUmUmCmUmCmUmUmAmUmUTCCC (SEQ ID NO: 7) | 4nt_Guide |
| mCmAmUmUmCmUmCmUmUmAmUTTCCmC (SEQ ID NO: 8) | 5nt_Guide |
| mCmAmUmUmCmUmCmUmUmATTTCmCmC (SEQ ID NO: 9) | 6nt_Guide |
| mCmAmUmUmCmUmCmUmUATTTmCmCmC (SEQ ID NO: 10) | 7nt_Guide |
| mCmAmUmUmCmUmCmUTATTmUmCmCmC (SEQ ID NO: 11) | 8nt_Guide |
| mCmAmUmUmCmUmCTTATmUmUmCmCmC (SEQ ID NO: 12) | 9nt_Guide |
| mCmAmUmUmCmUCTTAmUmUmUmCmCmC (SEQ ID NO: 13) | 10nt_Guide |
| mCmAmUmUmCTCTTmAmUmUmUmCmCmC (SEQ ID NO: 14) | 11nt_Guide |
| mCmAmUmUCTCTmUmAmUmUmUmCmCmC (SEQ ID NO: 15) | 12nt_Guide |
| mCmAmUTCTCmUmUmAmUmUmUmCmCmC (SEQ ID NO: 16) | 13nt_Guide |
| mCmATTCTmCmUmUmAmUmUmUmCmCmC (SEQ ID NO: 17) | 14nt_Guide |
| mCATTCmUmCmUmUmAmUmUmUmCmCmC (SEQ ID NO: 18) | 15nt_Guide |
| mUTATTmUmCmCmC (SEQ ID NO: 19) | L=9 8nt Guide |
| mUmUATTTmCmCmC (SEQ ID NO: 20) | L=9 7nt Guide |
| +UTTTT+U+C+C+C (SEQ ID NO: 21) | L=9 8nt LNAguide |
| +U+UATTT+C+C+C (SEQ ID NO: 22) | L=9 7nt LNAguide |
| mUTATTmU (SEQ ID NO: 115) | L=6 9nt Guide |

TABLE 6

Example RNase H digestion protocol

| Component | | Units | Concentration | µL | |
|---|---|---|---|---|---|
| | mRNA | ng/µL | 1000 | 20 | |
| IDT | chimeric oligo | mM | 1 | 1.45 | |
| | 65° C. for 5 min | | | | |
| Epicentre | RNase H (10 U) | U/µL | 5 | 2 | at 5000 U/mL |
| NEB | 10x RNase H buffer | | 10X | 3 | <Contains DTT and MgCl2 |
| | Total Volume | | | 26.5 | |
| NEB | CIP | U/µL | 2 | 2 | |
| | Total volume | | | 28.5 | |
| | 37° C. for 1 hr | | | | |
| | 250 mM EDTA, 1 M TEAA | | | 5 | |

In some embodiments, CIP facilitates a more consistent and reliable quantification of mRNA target fragments by normalizing all terminal 5′ and 3′ ends to hydroxyl groups. Thus, in some embodiments, the use of CIP provides more reliable and accurate LC-MS data analysis of mRNA cap/tail targets generated from RNase H guide directed site-specific activity than mRNA digestion protocols that omit CIP. In some embodiments, all components of step 1 and step 2 described in Table 6 above (e.g., mRNA, guide strand, RNase H, CIP, 10x buffer) are combined into a single reaction mixture and RNase H digestion is performed at 65° C. for 15 minutes (in the absence of an annealing step) followed by step 3 (reaction quenching). In some embodiments, one-pot RNase H digestion significantly shortens the total digestion time and decreases the total number of procedure steps, directly accommodating a high-throughput environment. In some embodiments, immediately after performing a one-pot RNase H digest, the reaction mixture can be directly injected into the LC-MS for analysis without the need for post-digest purification steps to remove the RNase H guides and/or digestion proteins. In some embodiments, the lack of a post-digest purification/work-up step (e.g., via biotin pull down assay) is a direct result of the one-pot assay design described by the disclosure, which provides suitable conditions with respect to RNase H guide length, target cap/tail fragment lengths and LC-MS analysis parameters (temperature, mobile phase, column).

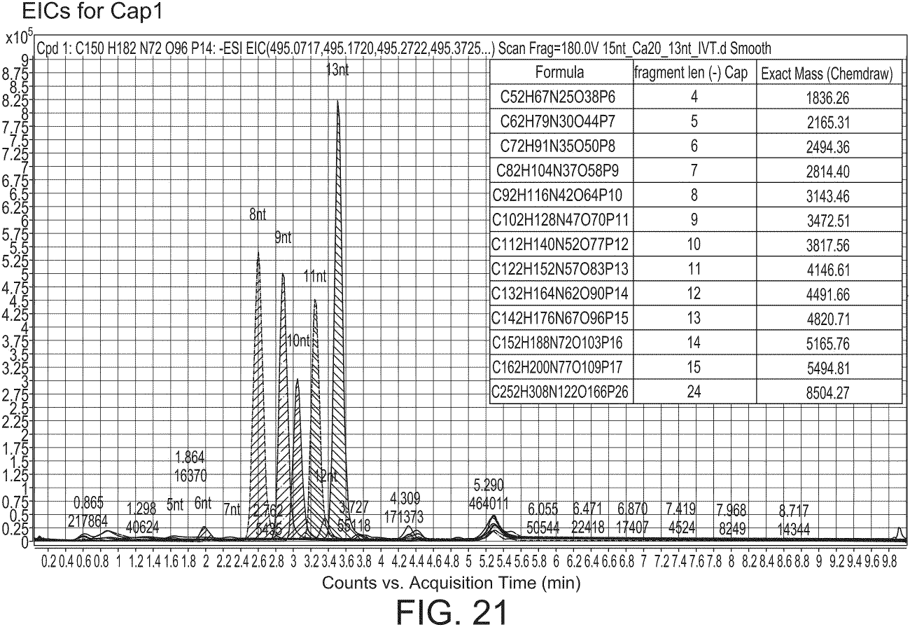

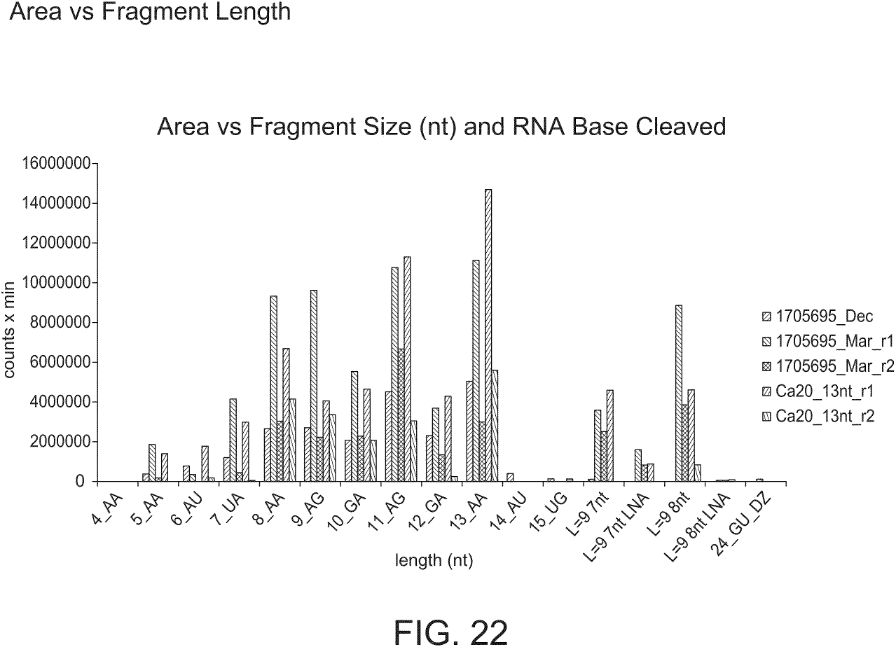

In some embodiments, RNase H cleavage position can vary based on the quality and supplier of the enzyme. In this example, thermostable RNase H, Hybridase (Epicentre, Illumina) was used. Specific cleavage consistently has been observed between the 2′O-methyl RNA flanking the final DNA base (designating the cut site) for variety of guides, allowing one to have control over the length of the resulting mRNA fragment (FIG. 21); this utility allows one to have full control over the length of the desired mRNA fragment generated from RNase H activity, which advances one’s ability to control and optimize the desired retention time of the target fragments generated by RNase H. Furthermore, FIG. 22 shows representative data of peak area versus fragment length (nt) for the mRNA Cap, digested with RNase H directed by guide strands targeting different RNase H sites and varying guide lengths. As observed, reducing guide strand length from 16 nt (“8_AA”) to 9 nt (“L9 8 nt”) does not significantly impact the signal of the resulting target Cap fragment as measured by mass spectrometer (MS). Therefore, accumulatively, having the ability to direct RNase H specificity and flexibility in the length of the RNase H guide strand significantly advances one’s ability to direct the retention times of the RNase H target fragment (e.g., cap fragment) and the RNase H guide itself, allowing one to prevent undesired co-elution, and consequently, yield relatively consistent reliable and clean LC-MS data. It should be noted that it is expected that in some cases, RNase H cleavage of mRNA may not total, but succeed in most cases where DNAzyme fails. Furthermore, guide strands can be designed to target any UTR of interest.

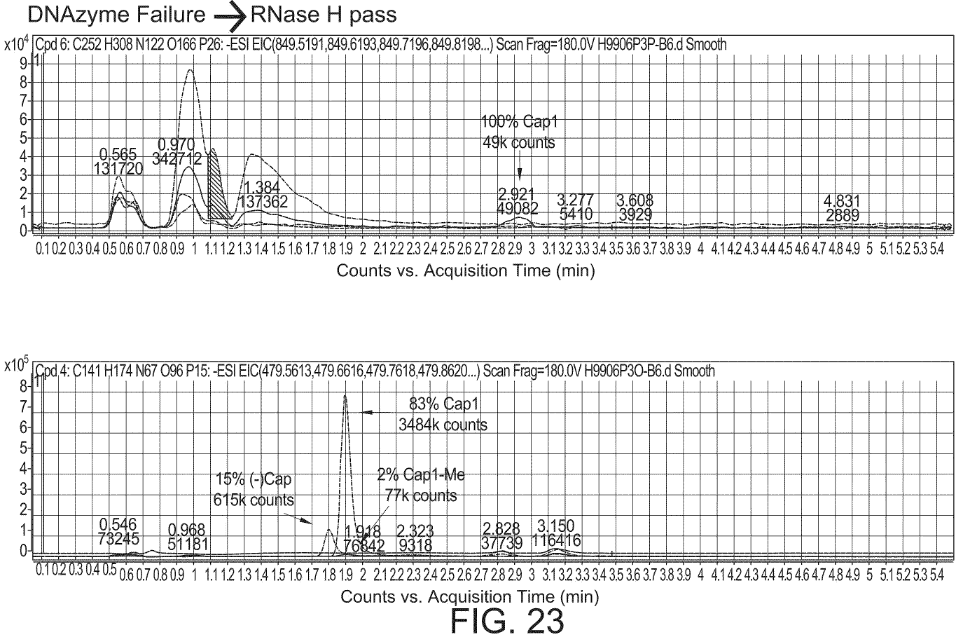

FIG. 23 shows representative MS data comparing mRNA Cap digestion by DNAzyme (top) and RNase H (bottom). For some constructs, DNAzyme does not cleave the 5′UTR efficiently, or at all. In these cases, RNase H has proven to be superior.

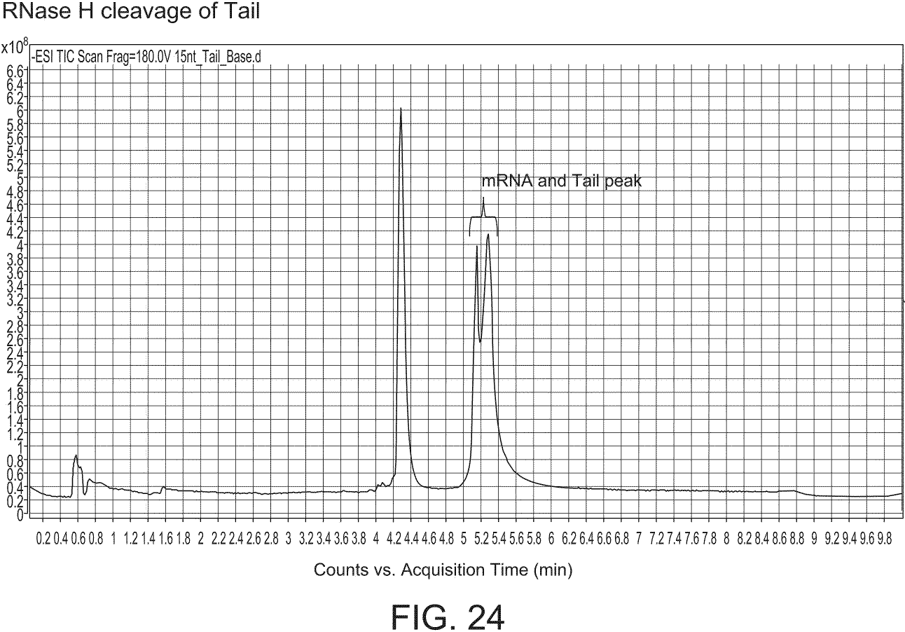

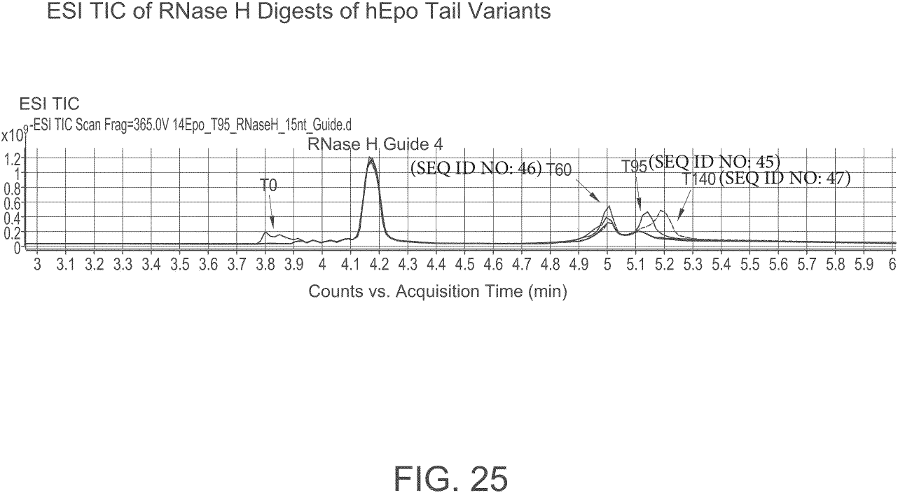

Similar to DNAzyme, after certain RNase H digestions, the undigested mRNA and some Tail species may co-elute due to the hydrophobicity of the polyA Tail (FIG. 24); this is highly subjective to the length of the target mRNA and the length of the target RNase H tail fragment, and currently does not compromise the ability to identify tail lengths that co-elute with undigested mRNA. In FIG. 25, the data indicate the potential co-elution of the current RNase H tail guide strand with targeted tail species that fall between lengths of 0 (“T0”) and 60 nucleotides (“T60”), which may bias quantitation of some Tail lengths; currently, this potential co-elution has been narrowed down to tail lengths between T0 and T20.

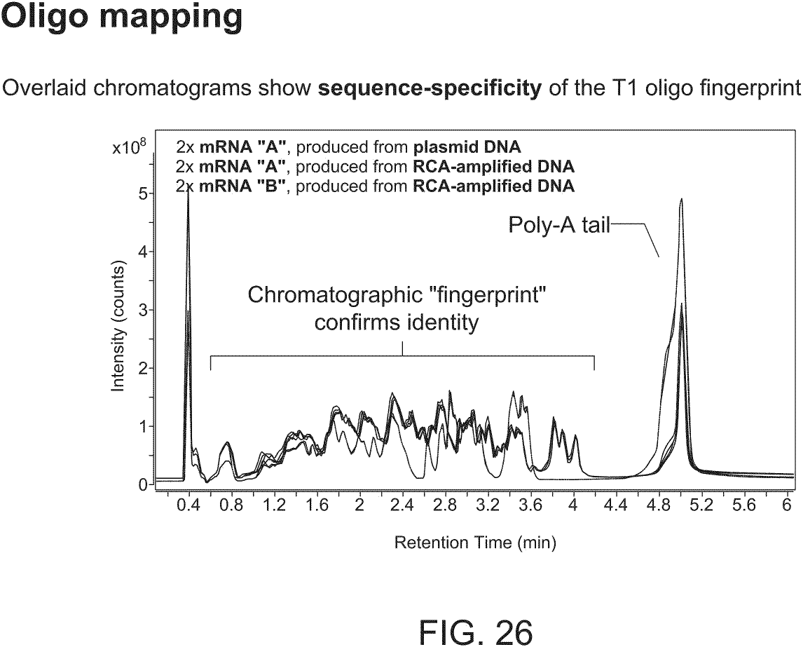

RNase T1 cuts to the 3′ of every canonical G and can be used for mRNA fingerprinting. FIG. 26 shows representative data relating to the sequence-specificity of RNase T1 mRNA fingerprinting. Chromatograms for three different mRNA (“mRNA A” produced from plasmid DNA, “mRNA A” produced from rolling circle amplification (RCA)-amplified DNA, and “mRNA B” produced from RCA-amplified DNA) were overlaid and chromatographic fingerprints were compared. Data indicate that after digestion with RNase T1, chromatographic fingerprints of the two “mRNA A”s are the same, while the “mRNA B” fingerprint is different.

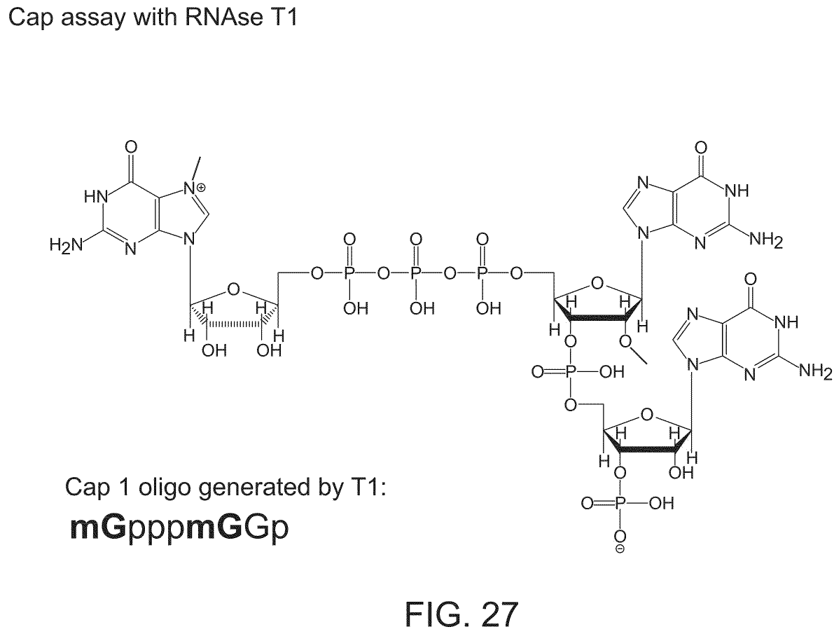

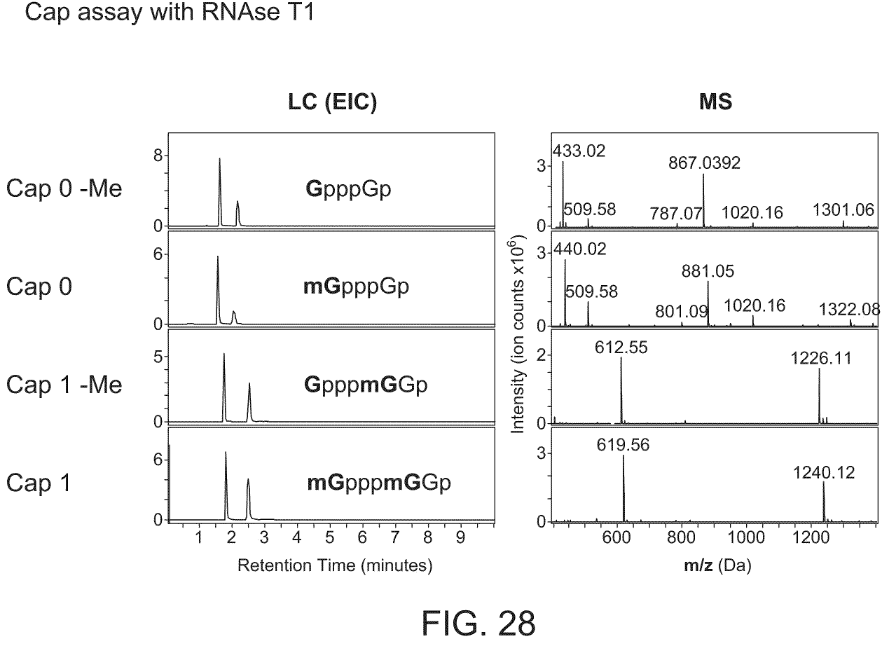

RNase T1 was also used to characterize mRNA Cap. FIG. 27 shows a schematic depiction of one embodiment of mRNA Cap digestion by RNase T1. FIG. 28 shows representative LC and MS data related to mRNA Cap digestion using RNase T1. Data indicate that RNase T1 digestion allows quantitation of four Cap subspecies as well as Uncapped mRNA.

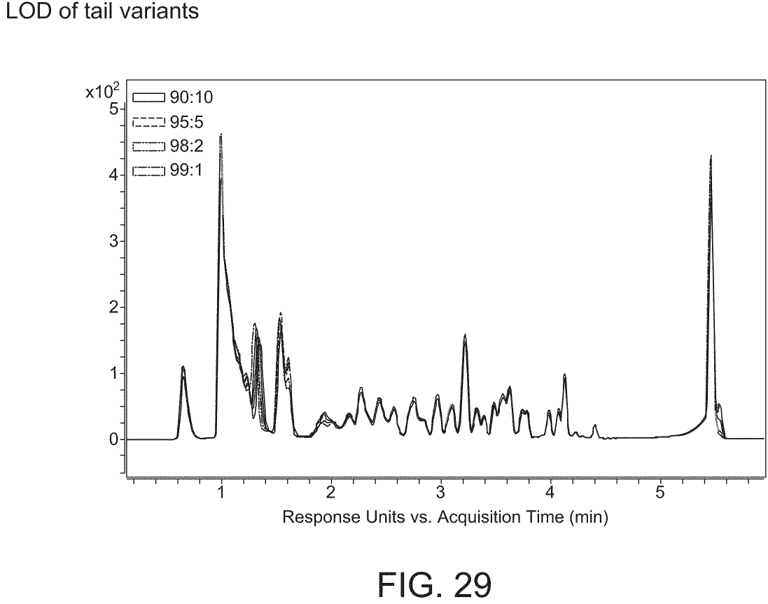

Tail length quantitation was also performed using RNase T1. FIG. 29 shows representative data related to the limit of detection (LOD) of mRNA tail variants by RNase T1 digestion. As the RNase T1 digestion progresses, secondary structure is removed, allowing the mRNA to be completely digested, allowing for accurate quantitation of the Tail. RNase A functions similarly to T1 cleaving 3′ of C and U, and sometimes A.

### 2. Design of Mrna Polya Tail Rnase H Guide Sequences

Guide strands for RNase H-based characterization of mRNA poly A Tail were designed. In this example, RNase H guide strands comprise the following generic formula:

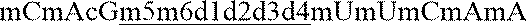

where the underlined portion of the formula comprises the DNA/RNA recognition motif identified to be required for specific RNase H (Epicenter) cleavage of a target mRNA; “m” denotes 2′O-methyl modified RNA and “d” denotes 2-deoxyribonucleotides. Non-limiting examples of RNase H tail guides are shown in Table 7.

TABLE 7

Non-limiting examples of RNase H Tail guide strands

| | | |
|---|---|---|
| Guide Strand Name | Sequence | Tail Cleavage? |
| Guide 1 | mUmUmUmUmUmUmUmUmUdTdGdCdCmGmCm CmCmAmCmUmCmAmG (SEQ ID NO: 23) | Yes |
| Guide 2 | mGmCmCmGmCdCdCdAdCmUmCmAmGmA (SEQ ID NO: 24) | Yes |
| Guide 3 | mCmCmAmCmUdCdAdGdAmCmUmUmUmA (SEQ ID NO: 25) | No |
| Guide 4 (T.T.T.A) | mCmAmGmAmCdTdTdTdAmUmUmCmAmA (SEQ ID NO: 26) | Yes |
| Guide 5 | mUmUmUmAmUdTdCdAdAmAmGmAmCmC (SEQ ID NO: 27) | Yes |
| T.T.T.A (short) | mGmAdCdTdTdTdAmUmUmC (SEQ ID NO: 28) | Yes |
| T.T.T.A + 3′6FAM | mCmAmGmAmCdTdTdTdAm Um UmCmAmA-36FAM (SEQ ID NO: 29) | Yes |
| T.T.T.A + 3′Sp18 | mCmAmGmAmCdTdTdTdAm Um UmCmAmA-3Sp13 (SEQ ID NO: 30) | Yes |
| N.T.T.A | mCmAmGmAmCdNdTdTdAmUmUmCmAmA (SEQ ID NO: 31) | No |
| T.N.T.A | mCmAmGmAmCdTdNdTdAmUmUmCmAmA (SEQ ID NO: 32) | No |
| T.T.N.A | mCmAmGmAmCdTdTdNdAmUmUmCmAmA (SEQ ID NO: 33) | Yes |
| T.T.T.N | mCmAmGmAmCdTdTdTdNmUmUmCmAmA (SEQ ID NO: 34) | Yes |
| N.N.N.N | mCmAmGmAmCdNdNdNdNmUmUmCmAmA (SEQ ID NO: 35) | No |
| I.T.T.A | mCmAmGmAmCdIdTdTdAmUmUmCmAmA (SEQ ID NO: 36) | No |
| T.l.T.A | mCmAmGmAmCdTdIdTdAmUmUmCmAmA (SEQ ID NO: 37) | No |
| T.T.I.A | mCmAmGmAmCdTdTdIdAmUmUmCmAmA (SEQ ID NO: 38) | Yes |
| T.T.T.I | mCmAmGmAmCdTdTdTdlmUmUmCmAmA (SEQ ID NO: 39) | Yes |
| N.mC.T.T.T.A | mCmAmGdNmCdTdTdTdAmUmUmCmAmA (SEQ ID NO: 40) | No |
| N.T.T.T.A | mCmAmGmAdNdTdTdTdAmUmUmCmAmA (SEQ ID NO: 41) | No |
| N.N.T.T.T.A | mCmAmGdNdNdTdTdTdAmUmUmCmAmA (SEQ ID NO: 42) | No |
| T.T | mCmAmGmAmCdTdTmUmAmUmUmCmAmA (SEQ ID NO: 43) | No |
| 4cuttertail | dNdNdNmAmCdTdTdNdNdNdNdNdNdN (SEQ ID NO: 44) | No |

N = 5-nitroindole; I = Inosine; m = 2′-O-methylated base; d = 2′-deoxyribonucleotide

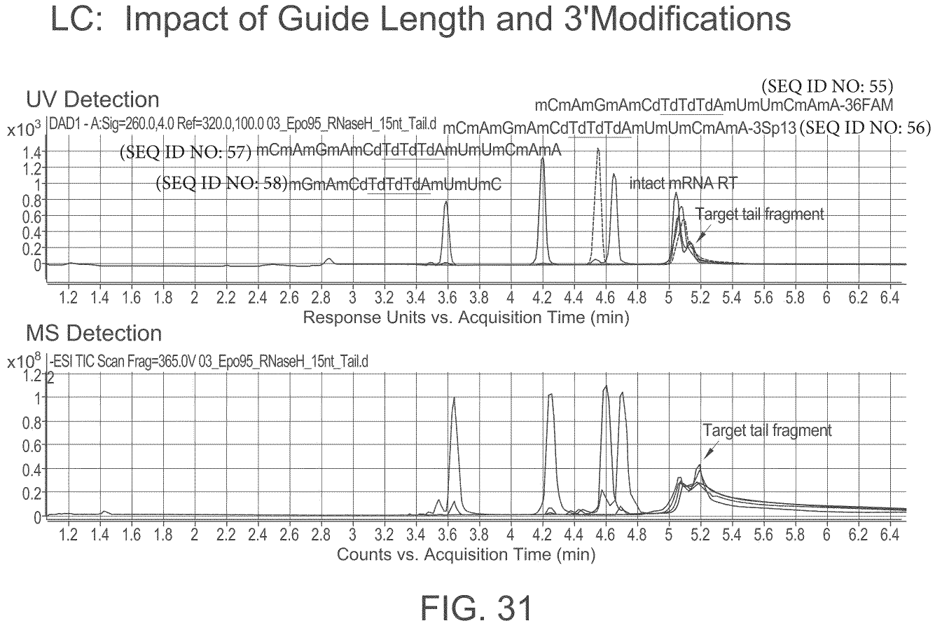

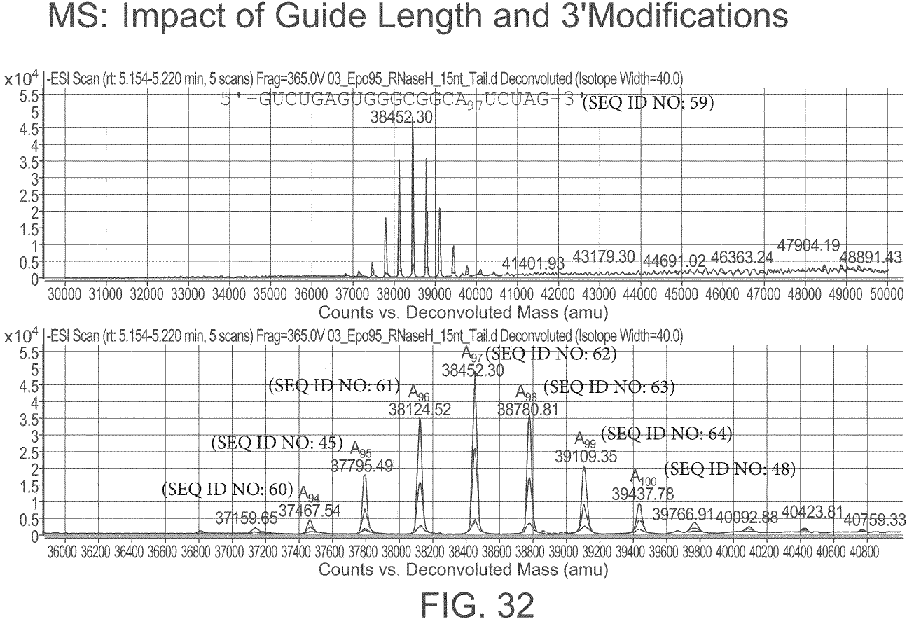

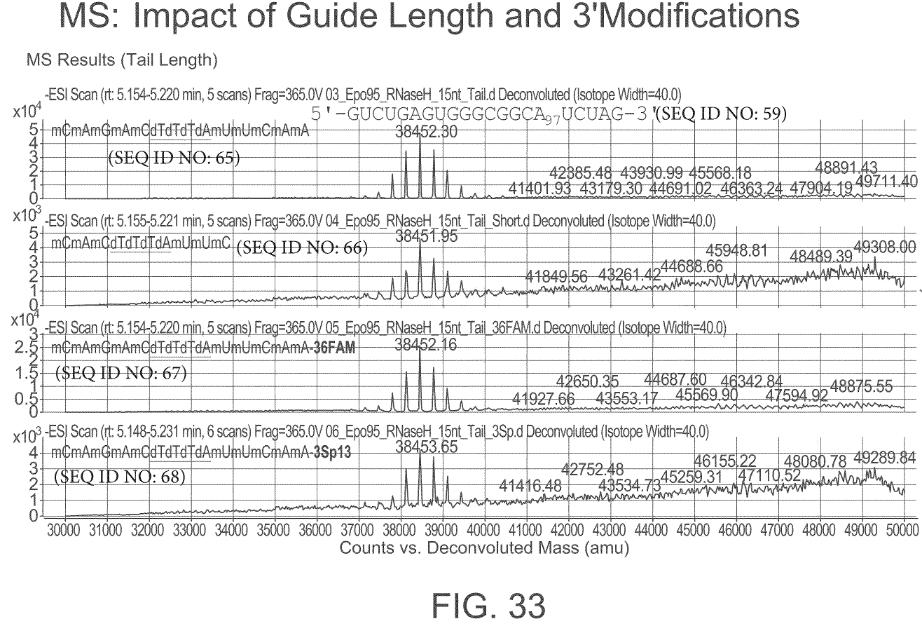

RNase H cleavage of mRNA Tail was tested for each of the guide strands listed in Table 7. FIGS. 31-33 show representative data illustrating the impact of RNase H guide strand length and 3′ modification on target tail fragment identification and relative quantitation by tandem liquid chromatography (LC) UV and MS detection. Data shown are for RNase H digestions directed by four guide strand variants of guide strand #4. Briefly, consistent with our previously reported observations with the RNase H cap guide designs, one can direct the retention times of the RNase H tail guides by altering strand length. Furthermore, this data highlights an additional innovative approach for directing RNase H guide retention time, which can also be done by modifying the 3′ terminus of the guide strand with a fluorescent moiety (e.g., 6FAM) or spacer molecule (Sp18) without compromising RNase H cleavage specificity and also without impacting the relative quantitation and identification of mRNA tail length by RNase H digestion.

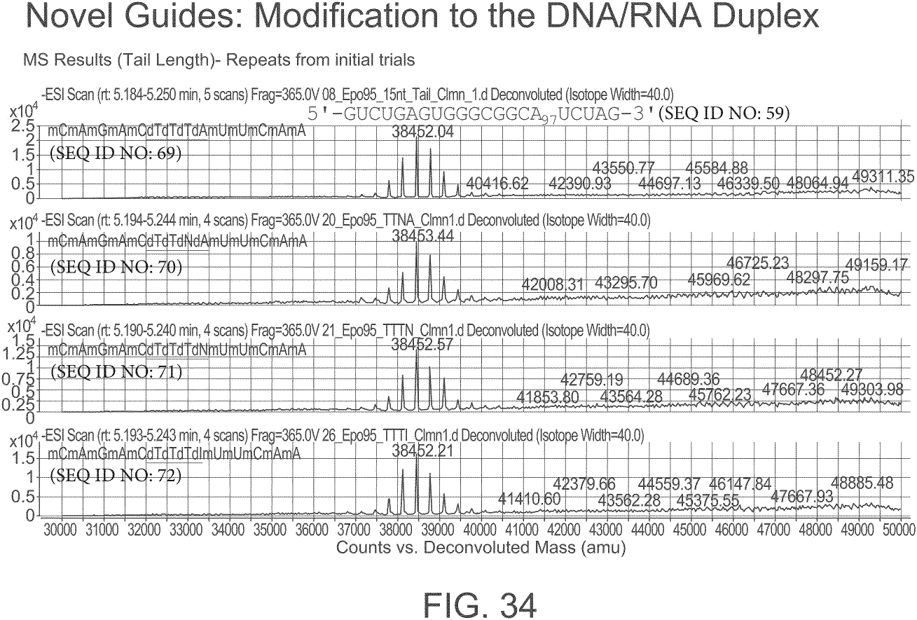

FIG. 34 shows representative data illustrating the impact of RNase H guide strand modification on mRNA tail length quantitation as measured by MS. Guide strands were modified by substitution of non-traditional nucleobases (5-nitroindole “N”, and Inosine “I”) at a site within the DNA/RNA recognition motif of the guide stand. Data indicate that nucleotides at positions d3 and d4 of the DNA/RNA recognition motif are not required to be traditional nucleobases and can be unconventional, as cleavage of target tail fragment is observed when these positions are non-traditional nucleobases. RNase H cleavage is not observed when positions d1 and d2 of the DNA/RNA recognition motif are non-traditional nucleobases, highlighting the essential contributions of traditional nucleobases in these positions for RNase H cleavage activity.

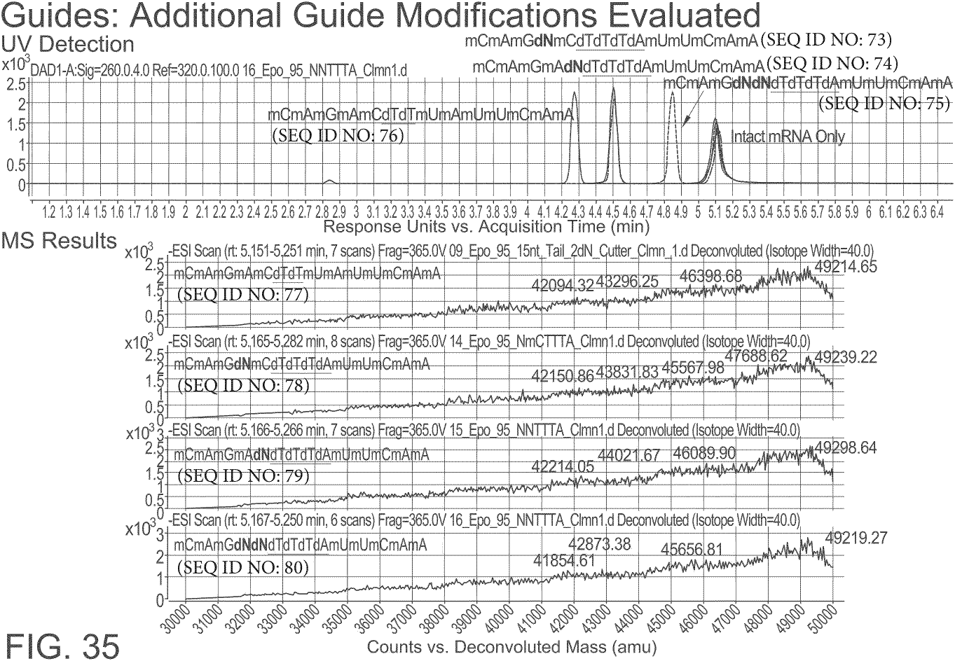

FIG. 35 shows further representative data illustrating the impact of RNase H guide strand modification on RNase H activity, inhibiting mRNA tail length identification and relative quantification by LC-MS. Guide strands were modified by the substitution of non-traditional nucleobases (5-nitroindole “N”, and Inosine “I”) at positions m5 and m6 of the guide stand. Data indicate cleavage does not occur when positions m5 or m6 are not a traditional 2′-deoxyribonucleotide, suggesting that traditional nucleobase-pairing interactions at these positions are important for RNase H recognition and/or RNase H activity.

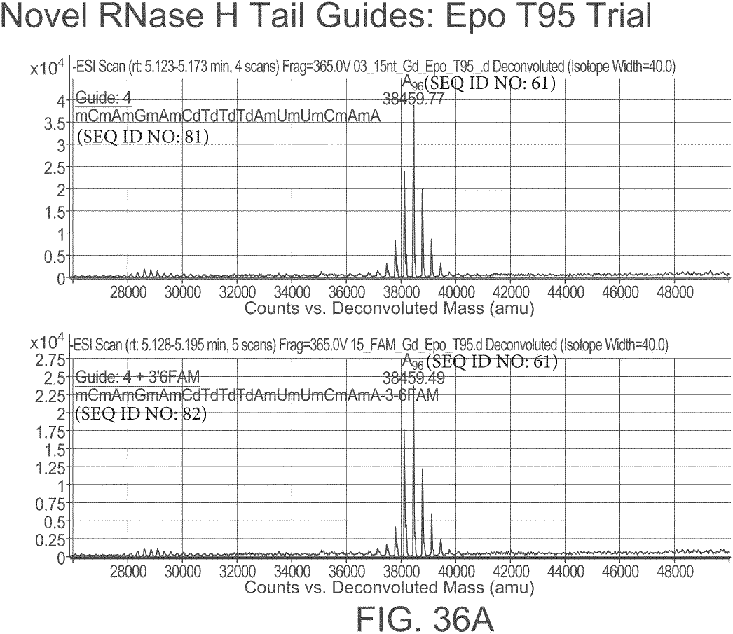

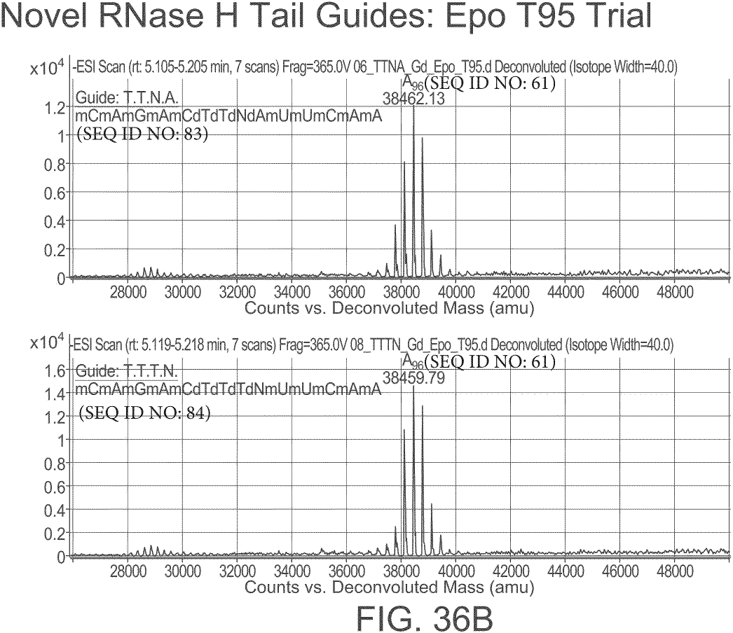

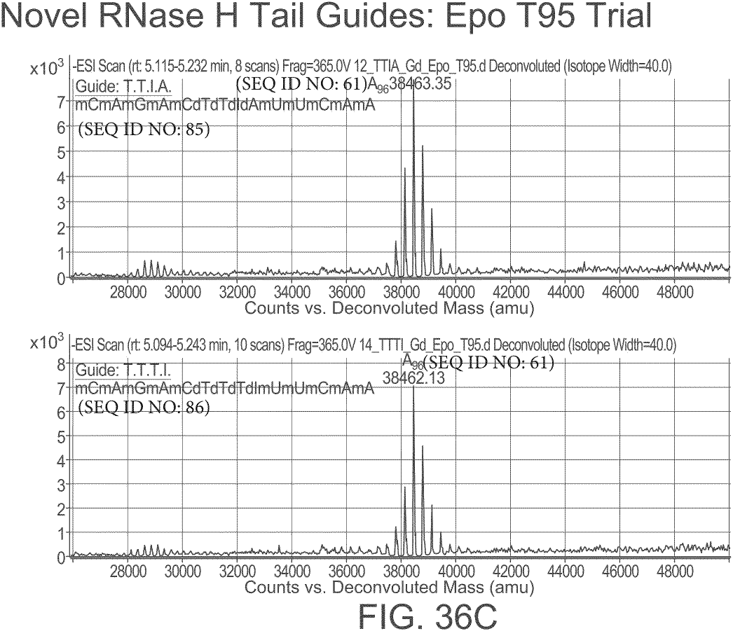

FIGS. 36A-36C show representative data illustrating RNase H guide strand modification on erythropoietin (Epo) mRNA tail length identification and quantitation as measured by LC-MS. The Epo mRNA digested has a theoretical tail length of 95 nucleotides (T95 (SEQ ID NO: 45)). FIG. 36A shows digestion of Epo T95 with RNase H Guide strand # 4 and a Guide strand #4 variant, which contains a 3′ 6-carboxyfluoroscein (3′-6FAM) modification. FIG. 36B shows Guide strand #4 variants, which contain a 5-nitroindole modification at position d3 (top) or d4 (bottom). FIG. 36C shows Guide strand #4 variants, which contain an Inosine modification at position d3 (top) or d4 (bottom). Accumulatively, RNase H digests done with these six different tail guides yield the same results for the tail length identification and relative quantitation of Epo T95 without compromising the integrity and specificity of RNase H activity.

Thus, in some embodiments, RNase H requires a DNA/RNA recognition motif that is > 2 base pairs in length for binding and cleavage specificity or activity is observed when m5m6dld2 are unmodified nucleobases.

### 3. Design of Open Reading Frame (ORF) RNase H Guide Sequences

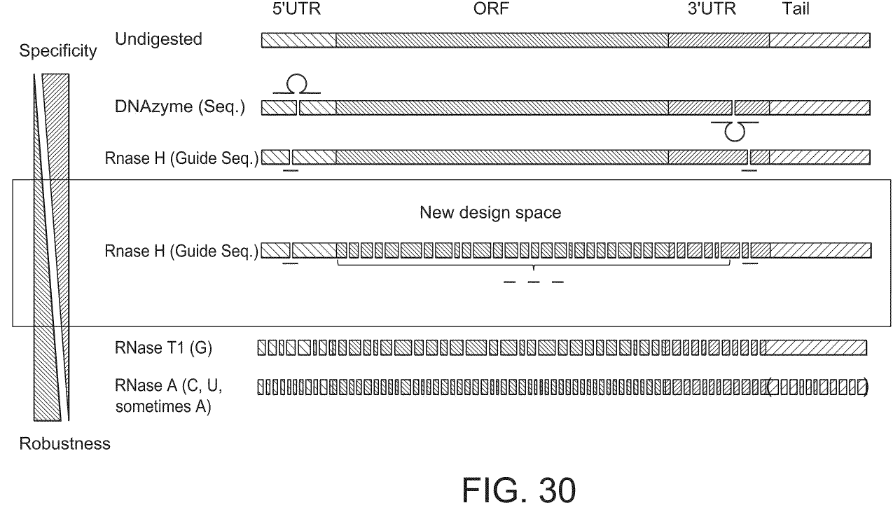

As described above, RNase H is a tunable tool for the digestion of mRNA Cap and Tail. This example describes the RNase H guide strands for cleavage of mRNA open reading frames (ORFs), as depicted in FIG. 30.

Cleaving the ORF will reduce secondary structure, similar to the activity of RNase T1, making targeted digestion for Cap and Tail fragments more complete. Generally, a single guide, or cocktail of guides that will give total ORF digestion similar to T1, but not interfere with targeted Cap and Tail digestion can be designed. This will allow for direct quantitation of all Cap and Tail species with less mRNA interference, the potential for mRNA mapping, and create a single pot digestion suitable for a high throughput environment.

Generally, thermostable RNase H has optimal activity between 65° C. and 95° C. Thus, cycling in a range between 37° C. and 95° C. allows for multiple binding and release of the guide stand(s) improving digestion efficiency and increasing the completeness of the digestion and enabling absolute quantitation.

Three concepts for ORF guides are described here: (1) short guides with four DNA bases flanked by two, one or zero 2′OMe RNA bases (e.g., mRDDDDmR, mRDDDD, DDDDmR, DDDD); (2) four DNA bases flanked by non-specific binding nucleotides of length to be determined (e.g., (N)_{q}DDDD(N)ₚ); and, (3) one, two or three DNA bases flanked by non-specific binding nucleotides, or a combination of 2′OMe RNA and non-specific nucleotides (e.g., (N)_{q}[quartet](n)ₚ, where [quartet] is all permutations and combinations of a total of four N′s and D′s). In the above examples, D = DNA, mR = 2′OMe RNA, N= non-specific nucleotide, p and q > 0, except in (3).

### Example 4: One Pot mRNA Cap/Tail Digest

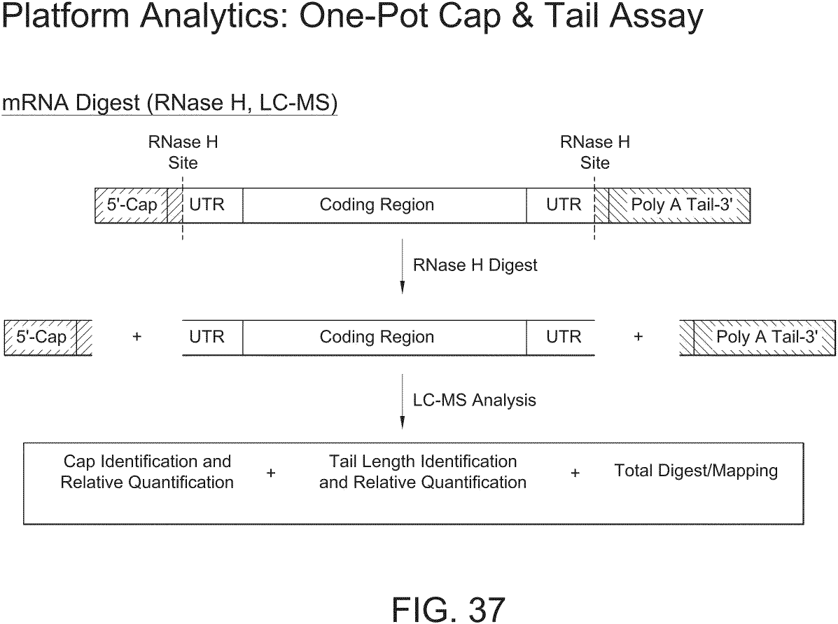

This example describes the simultaneous (e.g., one-pot) digestion of mRNA Cap and Tail region by RNase H. FIG. 37 shows a schematic depicting the mRNA digest protocol used in this example. Briefly, RNase H guide strands specific for Cap and Tail regions, but not specific for open reading frame (e.g., “coding region”) are used to digest an mRNA. LC-MS analysis is then performed and the following data are analyzed: (i) Cap identification and relative quantification; (ii) polyA tail length identification and relative quantification; optionally, (iii) total digest and mapping.

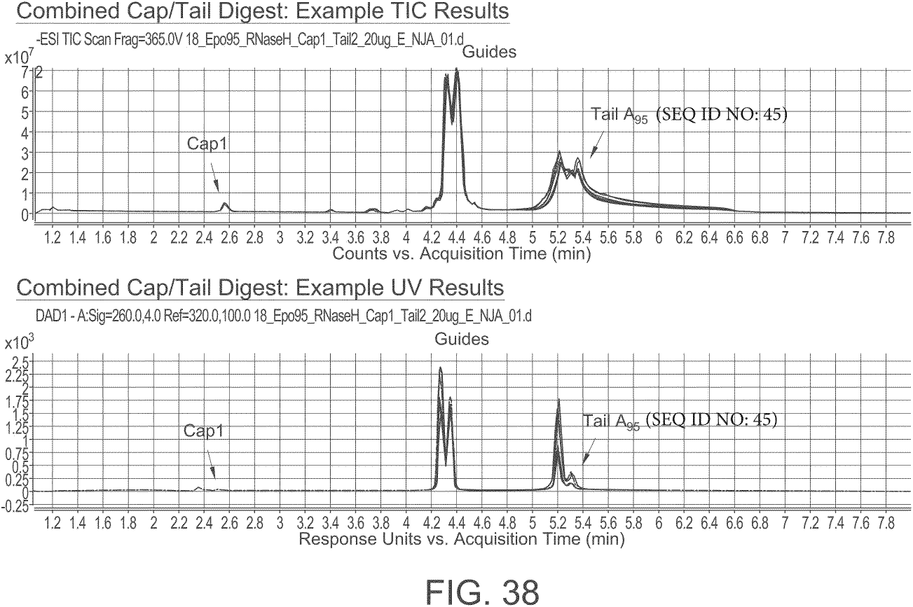

FIG. 38 shows representative data of mRNA Cap and tail one pot digestion using RNase H. The top panel of FIG. 38 shows analysis of combined Cap/tail digestion by total ion current chromatogram (TIC) and the bottom panel of FIG. 38 shows the same combined Cap/tail digest analyzed by UV detection.

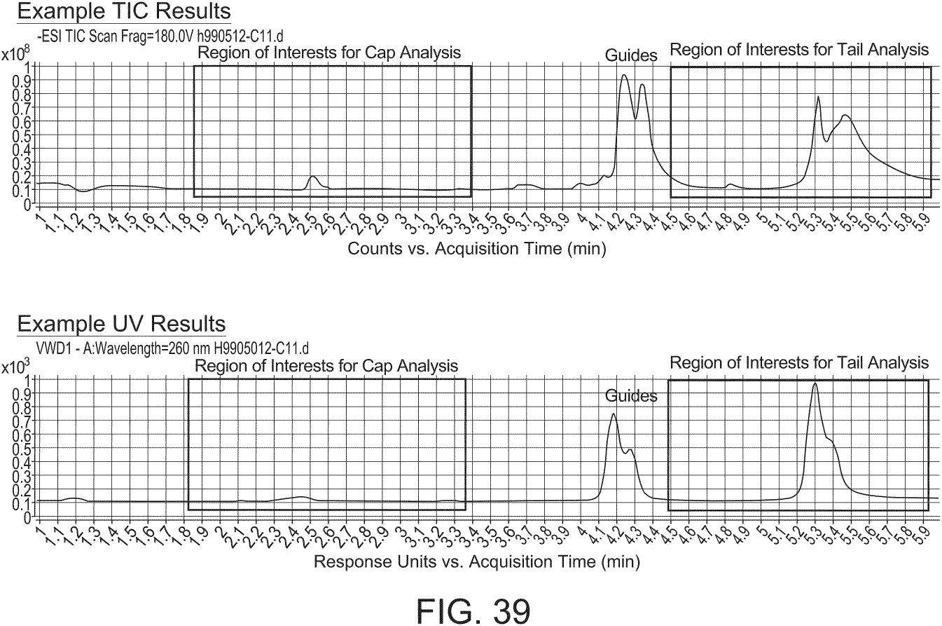

FIG. 39 shows representative quality control data for a combined Cap/tail one pot digestion. The top panel of FIG. 39 shows analysis by TIC and the bottom panel shows analysis by UV detection.

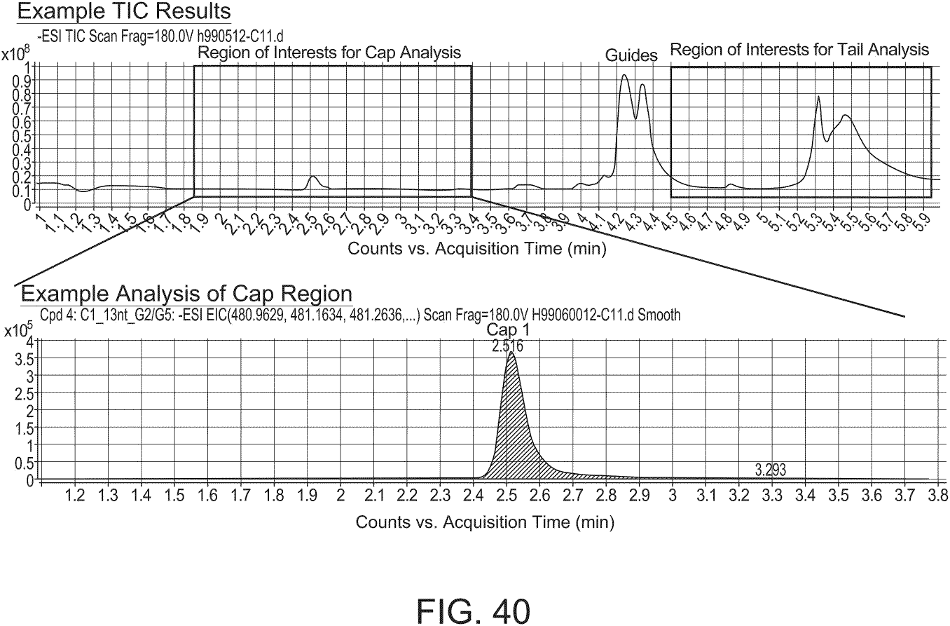

FIG. 40 shows representative data for the analysis of the Cap region of interest as identified by TIC. A single peak corresponding to Cap1 (e.g., complete 5′ Cap) was identified, indicating this mRNA is fully capped with the desired cap species.

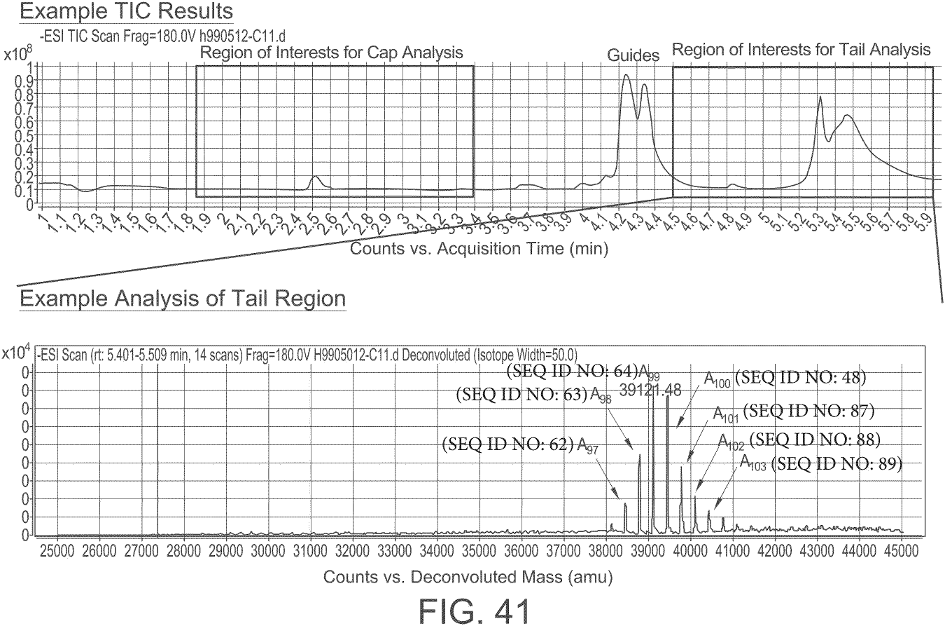

FIG. 41 shows representative data for the analysis of tail region of interest as identified by TIC. Table 8 provides representative data relating to detailed analysis of tail length. For this mRNA construct, the target tail length was T₁₀₀ (SEQ ID NO: 48) (a.k.a., A₁₀₀ (SEQ ID NO: 48)). The tail length observed using the Cap/tail one-pot digest indicates a tail length ranging from A₉₇-A₁₀₃ (SEQ ID NO: 112), indicating the presence of several tail variants near the target length of A₁₀₀ (SEQ ID NO: 48).

TABLE 8

| Tail Length | Calc | Obs | Area | Diff | Tail % |
|---|---|---|---|---|---|
| A97 (SEQ ID NO: 62) | 38457.96 | 38463.41 | 310668 | 5.450 | 7.386791 |
| A98 (SEQ ID NO: 63) | 38787.16 | 38792.7 | 657778 | 5.540 | 15.63906 |
| A99 (SEQ ID NO: 64) | 39116.37 | 39121.48 | 856936 | 5.108 | 20.37411 |
| A100 (SEQ ID NO: 48) | 39445.58 | 39451.16 | 864844 | 5.582 | 20.56218 |
| A101 (SEQ ID NO: 87) | 39774.78 | 39784.45 | 713833 | 9.666 | 16.9718 |
| A102 (SEQ ID NO: 88) | 40103.99 | 40111.97 | 451133 | 7.981 | 10.72595 |
| A103 (SEQ ID NO: 89) | 40433.20 | 40441.16 | 350784 | 7.965 | 8.340097 |
| | | | 4205994 | | 100 |

### Example 5: Investigation and Applications of RNase H for mRNA Cap and Tail Characterization

Characterization of mRNA quality attributes is, in some embodiments, important for the quality control of mRNA therapeutics. Two key components of mRNA stability and expression are the 5′ and 3′ terminal ends, which contain the 5′ cap and 3′ poly (A) tail. Here, a one-pot endonuclease digest coupled with Liquid Chromatography-Mass Spectrometry (LC-MS) analysis to determine the percent of functional cap and tail length of mRNA in a high-throughput environment is described.

RNase H guide strands specific for Cap and Tail regions, but not specific for open reading frame (e.g., “coding region”) were used to digest an mRNA encoding human EPO (hEPO). LC-MS analysis was then performed and the following data were analyzed: (i) polyA tail length identification and relative quantification; (ii) cap identification and relative quantification; and, (iii) substrate dependent RNase H activity in the context of the cap assay.

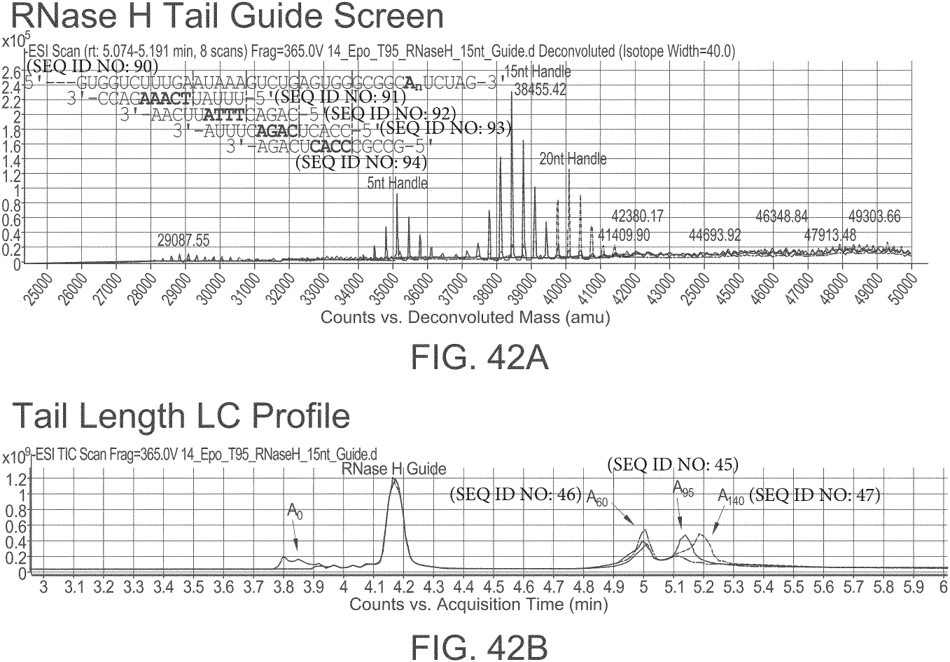

Data indicate that RNase H digestion guided by tail-specific guide strands allows for identification of tailless and Aₙ tail lengths, as well as “handle” sequences. FIGS. 42A-42B show representative data related to Poly(A) tail assay development. FIG. 42A shows representative LC-MS data of hEPO (theoretical tail length of A₉₅ (SEQ ID NO: 45)) interrogating RNase H activity with four different tail guides. Tail guides were designed to target the 3′UTR, allowing for tailless and An tail lengths to be identified. FIG. 42B shows representative LC profile (TIC) generated for hEPO with different theoretical tail lengths. Overlays of RNase H digestion products for tail lengths of A₀ (tailless), A₆₀ (SEQ ID NO: 46), A₉₅ (SEQ ID NO: 45) and A₁₄₀ (SEQ ID NO: 47) are shown.

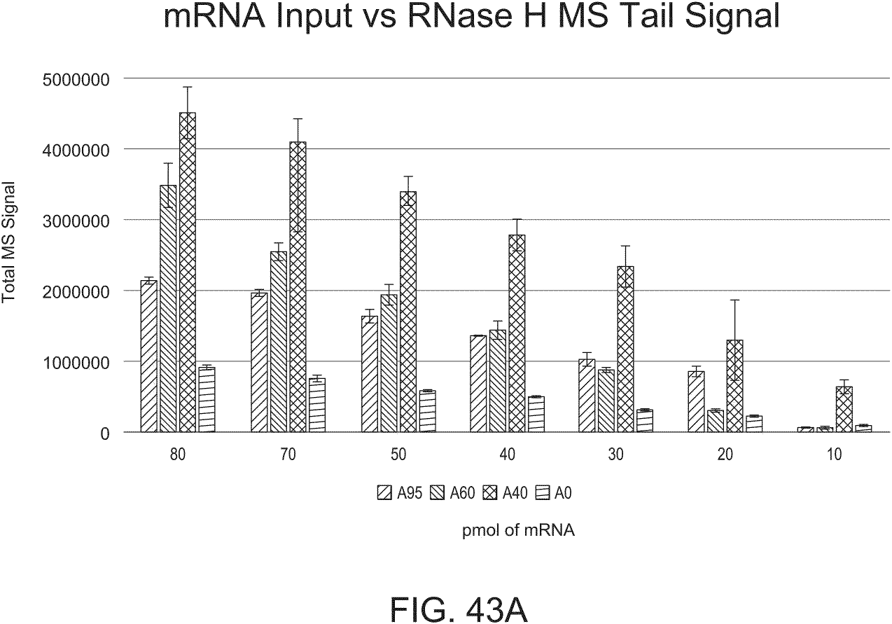

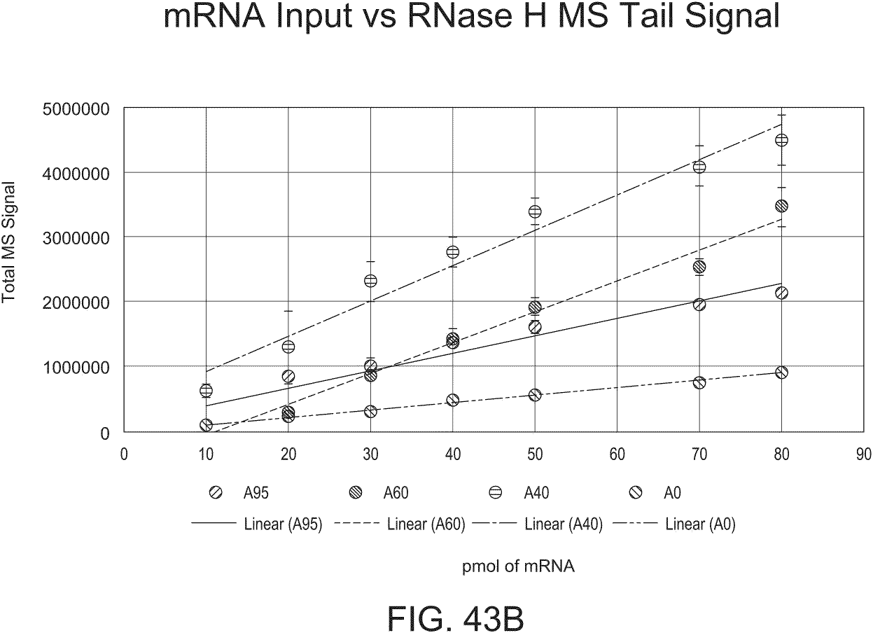

FIGS. 43A-43B show representative data related to evaluation the impact of mRNA tail length on MS signal. FIG. 43A demonstrates the relationship between MS signal and molar input of mRNA obtained for four different tail lengths (A95 (SEQ ID NO: 45), A60 (SEQ ID NO: 46), A40 (SEQ ID NO: 113), A0). FIG. 43B shows the linear relationship between total MS signal and molar input of each tail variant.

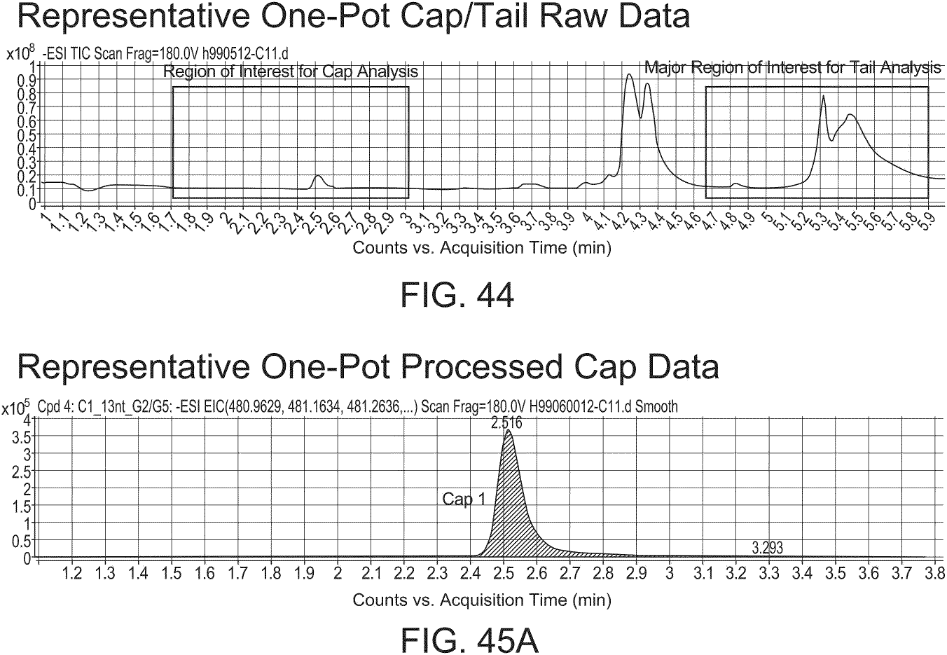

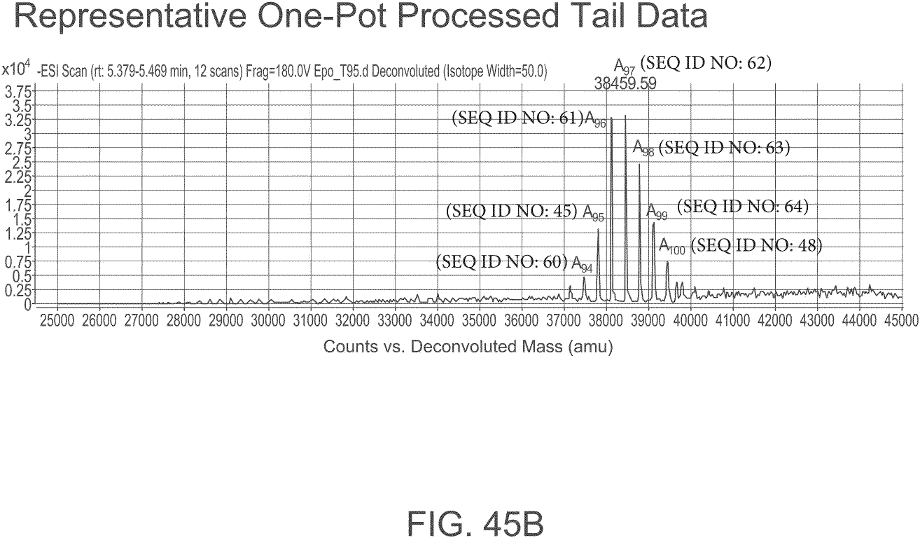

FIG. 44 shows representative raw data for a total ion chromatogram (TIC) of a one-pot cap/tail RNase H assay. The box on the left side of the histogram highlights the retention time region of interests for the cap variants, while the box on the right side of the histogram indicates the major region of interest for the tail analysis. Not shown in the target region where tailless elutes (3.0-3.2 mins). FIG. 45 A shows representative data for an extracted ion chromatogram (EIC) for the target cap variants. In this sample, only Cap 1 was identified. FIG. 45B shows representative deconvoluted MS data of the one-pot cap/tail RNase H assay for determining Poly (A) tail length. The different tail lengths are shown. This mRNA has a tail variants ranging from A₉₄-A₁₀₀ (SEQ ID NO: 114) in length.

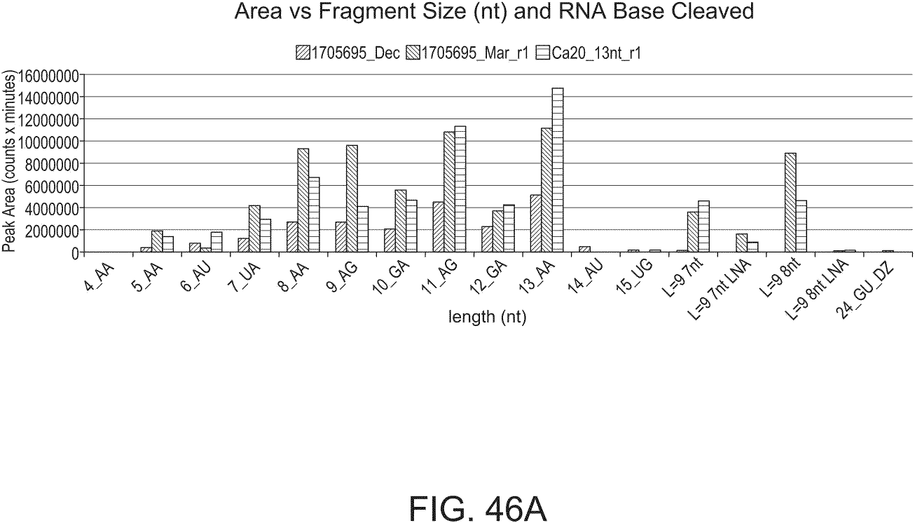

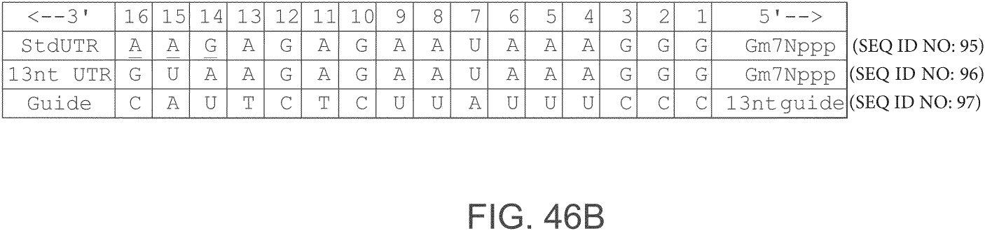

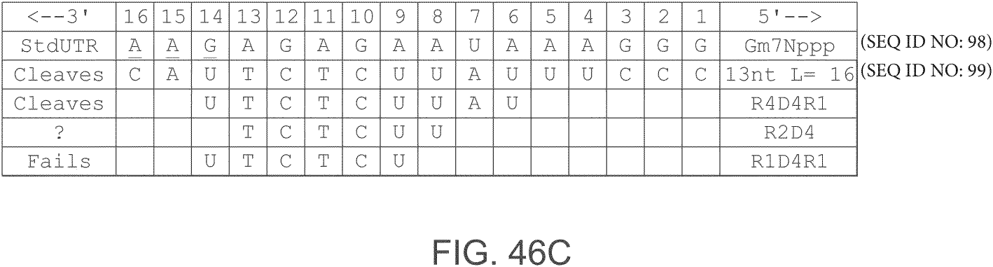

Next, RNase H substrate specificity was examined. Briefly, guide strands of varying length or of standard length but varying composition (e.g., with respect to nucleobase modifications) were tested. Cleavage efficiency of RNase H relative to RNA bases 5′ and 3′ of the cut site was evaluated. Data indicate that RNase H prefers to cut after A, and before A or G (FIG. 46A). In some embodiments, Uridine, modified in this case, prevents cleavage 3′ of the cut site, but only inhibits 5′ of the cut site.

FIG. 46B depicts an alignment of an example 5′ cap UTR with a 13-nucleotide shortened version and the most efficient RNase H guide strand identified in this example. The alignment indicates that 2′OMe bases (shown in italic) mismatched (shown in bold) to the 3′ of the cut site do not have an effect on RNase H cleavage. Additionally, data indicate that RNase H guides show efficacy with 3′ mismatches and there is no evidence that nearest neighbors to the cut site play a role in determining cleavage efficiency. Thus, shortened guide strands can be designed (FIG. 46C).

In sum, data indicates that RNase H has a consistent pattern of cleavage efficiency regardless of nearest neighbor effects and base mismatches. This indicates the characteristics which restrict RNase H+ Guide systems are located near the cut site, and distal regions may be modified or removed to decrease specificity or add other functionality. Furthermore, for a large number of constructs with different UTRs, shorter guides allow for cheaper, faster, purer guide synthesis.

### Example 6: Blocking Oligonucleotides

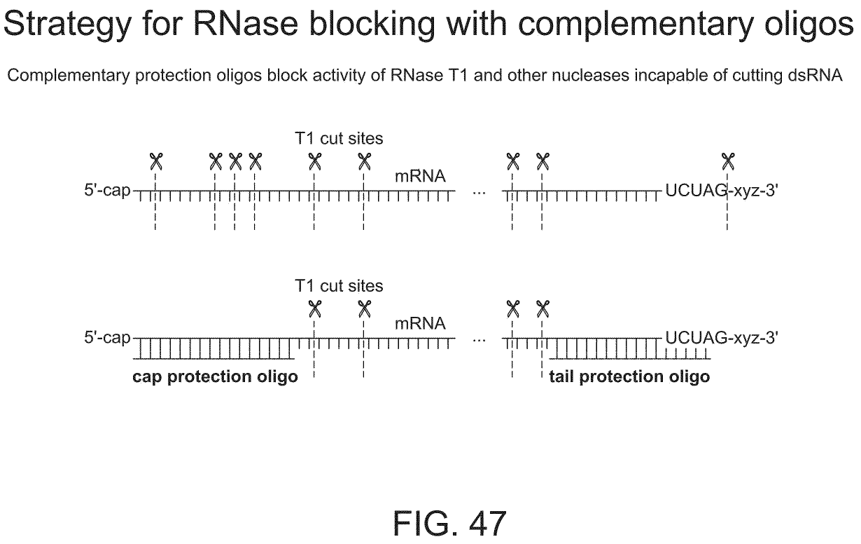

This example describes the use of blocking oligonucleotides to increase the cleavage repertoire of RNase (e.g., RNase T1) digestion of mRNA. Generally, blocking oligos are short oligonucleotide sequences that bind to a target site of an mRNA and prevent cleavage of the target site by an RNase, such as RNase T1, or other nucleases that cleave dsRNA. Blocking oligos are used, in some embodiments, to protect the 5′ end (e.g., the 5′ cap region) and/or the 3′ end (e.g., polyA tail region)of an mRNA from RNase cleavage (FIG. 47).

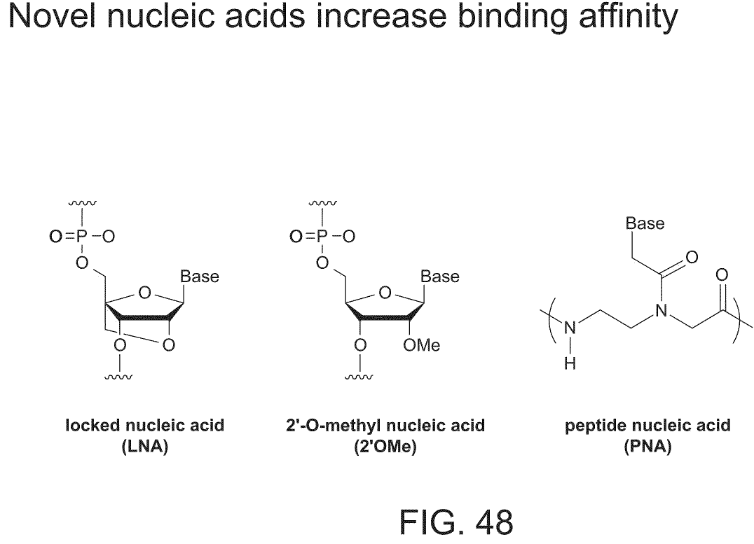

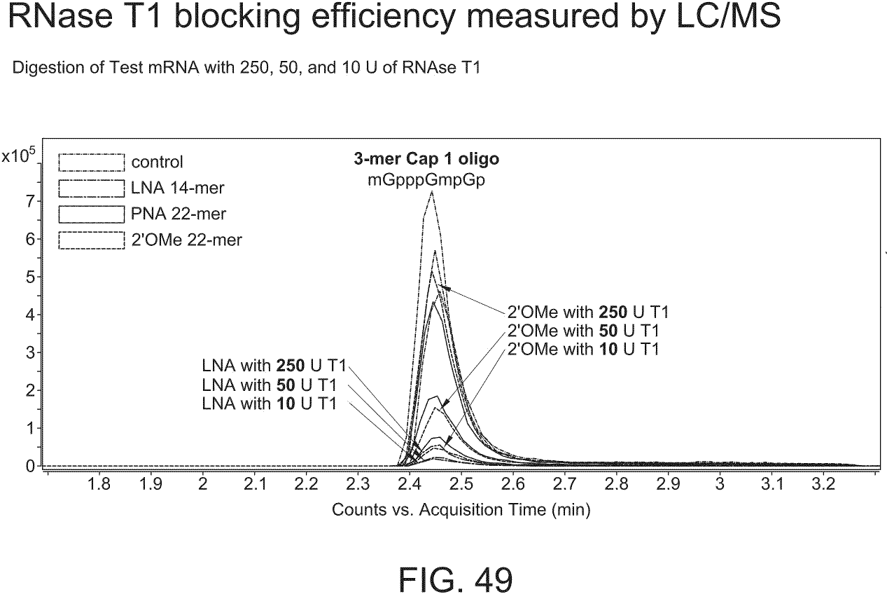

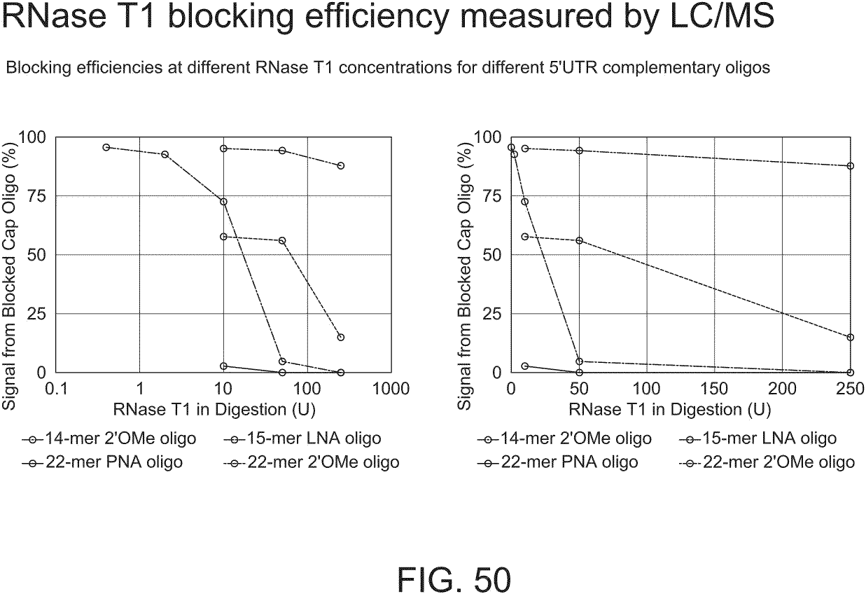

Blocking oligos (14-mer or 22-mer) having modified nucleic acids that increase oligo binding affinity were produced (FIG. 48). FIG. 49 shows representative data for RNase T1 blocking efficiency by modified nucleic acid (LNA, PNA, 2′ OMe) blocking oligos as measured by LC/MS. Briefly a target mRNA was digested with 250, 50, or 10 Units (U) of RNase T1 in the presence of LNA 14-mer blocking oligo, PNA 22-mer blocking oligo, or 2′OMe 22-mer, and compared to mRNA digested with RNase T1 in the absence of blocking oligo. Reduction in RNase T1 cleavage was observed for mRNA digested in the presence of blocking oligos compared to control. FIG. 50 shows representative data for RNase T1 blocking efficiency at different concentrations of RNase T1 by modified nucleic acid (LNA, PNA, 2′ OMe) blocking oligos as measured by LC/MS.

### Example 7: Bottom Up mRNA Mapping

This example describes sequence mapping of a test mRNA using RNase-based digestion of the mRNA sample and comparison of the resulting oligo signature profile with an in silico-produced control signature profile. Briefly, a test mRNA is digested using RNase (e.g., RNase T1, RNase H, etc.) into unique mass oligos, isomeric unique sequence oligos, or repetitive sequence oligos. Unique mass oligos may be identified, for example by LC-MS. Isomeric unique sequence oligos may be identified, for example by LC-MS/MS. Analysis of repetitive sequence oligos may be complemented via alternative enzymes.

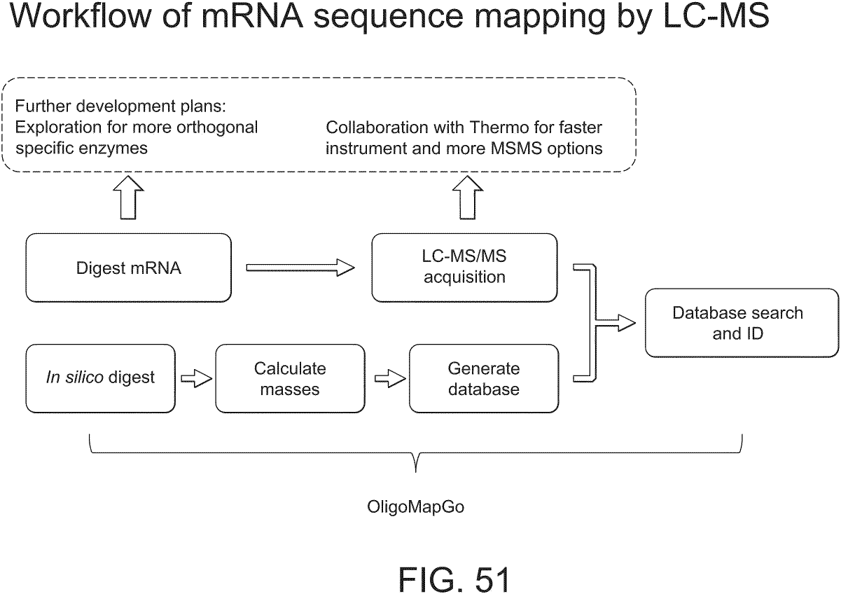

FIG. 51 shows a schematic depiction for one example of a mRNA sequence mapping workflow. Briefly, test mRNA is digested with RNase and analyzed via LC-MS/MS acquisition; in parallel, an in silico digest of a known control mRNA (e.g. the expected sequence of the test mRNA) is performed, fragment masses are calculated and a database of fragment masses is compiled. The results of the LC-MS/MS acquisition are then searched against the compiled database.

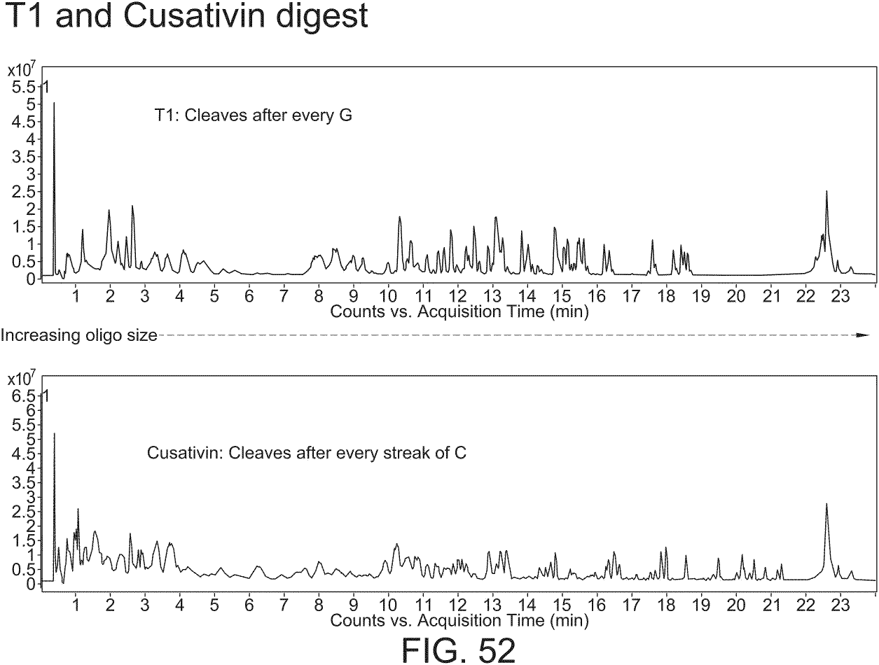

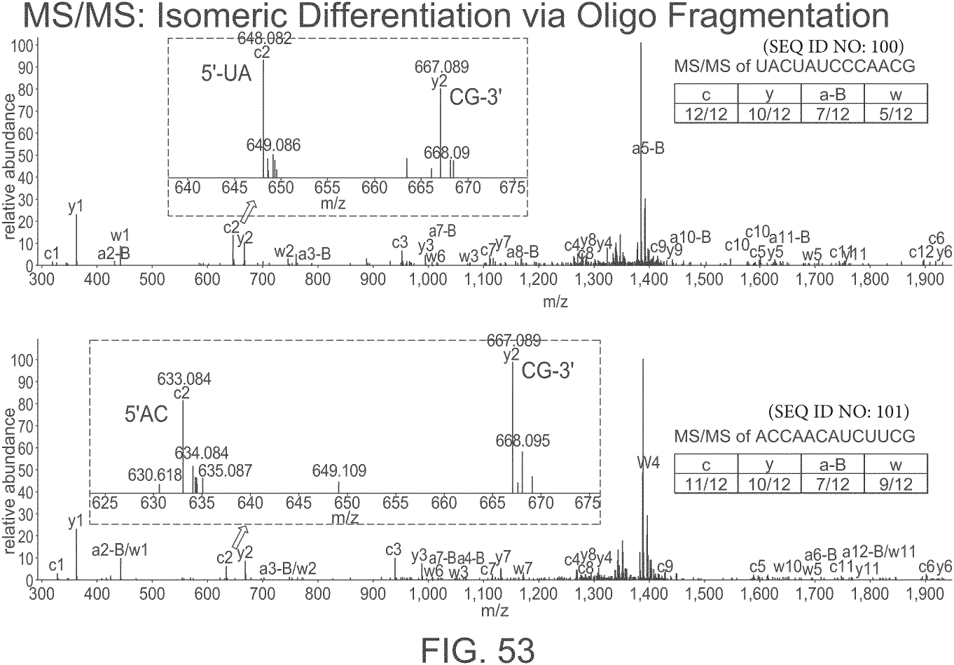

FIG. 52 shows examples of test mRNA digestion using RNase T1 (which cleaves RNA after each G) and Cusativin (which cleaves RNA after poly-C) in parallel (separate digestions). FIG. 53 shows examples of data produced by MS/MS isomeric differentiation via oligo fragmentation.

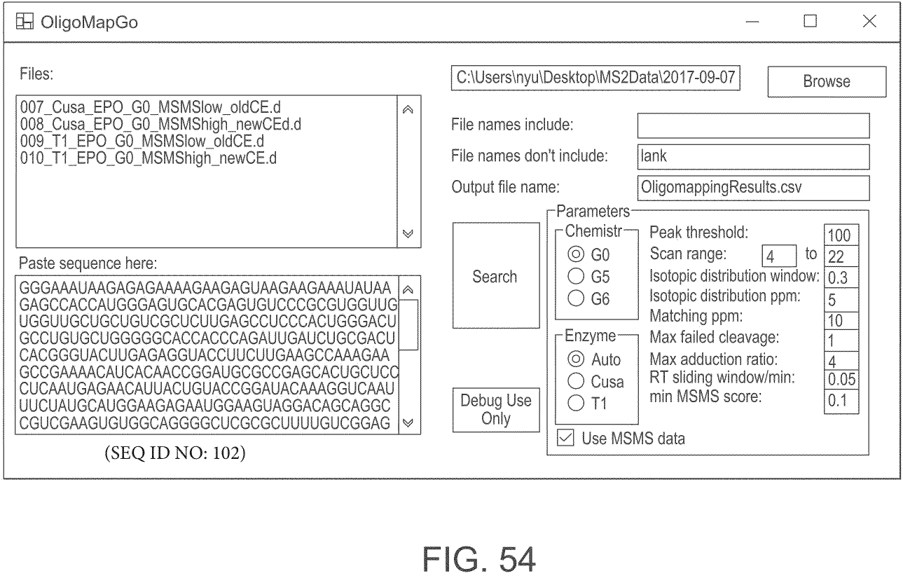

FIG. 54 shows an example of a graphic user interface (GUI) for the mRNA LC-MS/MS search engine. Briefly, in silico digestion is performed up to a specified number of failed cleavages. Resolved-isotopes deconvolute MS spectra by 3-second windows. The oligo compound is identified by mass and isotopic distribution and potential sodium and N,N-Diisopropylethylamine (DIEA) adduct false positives are removed. MS/MS data is checked for differentiation of isomers. Sequence coverage is then calculated. In auto-enzyme mode, sequence coverage is derived by union of coverage of each enzyme.

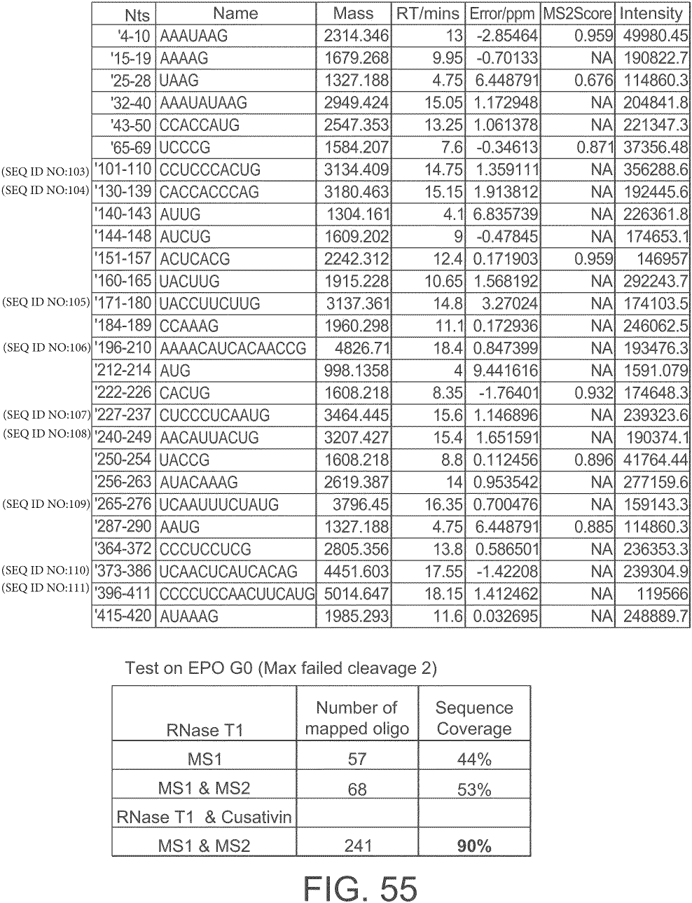

To determine if a MS/MS spectrum is matching an oligo, scoring function(s) and MS/MS spectrum filters are employed. FIG. 55 shows one example of calculation of the scoring function. An example of the output for LC-MS/MS sequence mapping.

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

1-119. (canceled)

120. A method for confirming identity of a test mRNA in a pharmaceutical composition, the method comprising:
digesting the test mRNA, by contacting the pharmaceutical composition with an endonuclease, to produce a plurality of mRNA fragments;
physically separating the plurality of mRNA fragments;
assigning a signature profile to the test mRNA by detecting the plurality of mRNA fragments; and
confirming the identity of the test mRNA if the signature profile of the test mRNA shares identity with the known mRNA signature profile.

121. The method of claim 120, wherein the test mRNA is in vitro transcribed (IVT) mRNA.

122. The method of claim 120, wherein the test mRNA encodes a vaccine antigen or a therapeutic protein or peptide.

123. The method of claim 120, wherein the endonuclease is an RNase H, RNase T1, cusativin, MazF, or colicin E5 enzyme.

124. The method of claim 120, wherein the endonuclease is an RNase T1 cusativin, MazF, or colicin E5 enzyme.

125. The method of claim 120, wherein the endonuclease is an RNase H.

126. The method of claim 125, wherein the digesting step further comprises, prior to contacting the pharmaceutical composition with the RNase H, contacting the pharmaceutical composition with:
(i) a guide RNA represented by the formula, from 5′ to 3′:
[R]_{q}D₁D₂D₃D₄[R]ₚ
wherein each R is an unmodified or modified RNA nucleotide, each D is a DNA nucleotide, and each of q and p are independently an integer between 0 and 50; or
(ii) a composition comprising a plurality of the guide RNAs, each represented by the formula of (i),
wherein hybridization of the guide RNA to an mRNA in the presence of RNase H enzyme results in cleavage of the mRNA by the RNase H.

127. The method of claim 126, wherein at least one R is a modified RNA nucleotide.

128. The method of claim 126, wherein at least one R is a 2′-O-methyl-modified RNA nucleotide.

129. The method of claim 126, wherein each R is modified RNA nucleotide.

130. The method of claim 126, wherein each R is a 2′-O-methyl-modified RNA nucleotide.

131. The method of claim 126, wherein each of D₁ and D₂ are unmodified DNA nucleotides.

132. The method of claim 126, wherein D₃, D₄, or both D₃ and D₄ are modified DNA nucleotides.

133. The method of claim 132, wherein the modified DNA nucleotide is 5-nitroindole, Inosine, 4-nitroindole, 6-nitroindole, 3-nitropyrrole, a 2-6-diaminopurine, 2-amino-adenine, or 2-thio-thiamine.

134. The method of claim 126, wherein hybridization of the guide RNA to the test mRNA in the presence of RNase H enzyme results in cleavage of the 5′ UTR of the test mRNA by the RNase H enzyme, and no cleavage of the ORF or 3′ UTR of the test mRNA.

135. The method of claim 126, wherein hybridization of the guide RNA to the test mRNA in the presence of RNase H enzyme results in cleavage of the test mRNA ORF by the RNase H enzyme, and no cleavage of the 5′ UTR or 3′ UTR of the test mRNA.

136. The method of claim 126, wherein hybridization of the guide RNA to the test mRNA in the presence of RNase H enzyme results in cleavage of the 3′ UTR of the test mRNA by the RNase H enzyme, and no cleavage of the 5′ UTR or ORF of the test mRNA.

137. The method of claim 120, wherein assigning the signature profile to the test mRNA comprises performing liquid chromatography-mass spectrometry (LC/MS) on the plurality of mRNA fragments.

138. The method of claim 120, wherein the known mRNA signature profile is determined by in silico sequence mapping.

139. The method of claim 120, wherein the identity of the test mRNA is confirmed if the signature profile of the test mRNA shares at least 80% identity with the known mRNA signature profile.
